(19) United States
(12) Patent Application Publication
(10) Pub. No.: US 2024/0043520 A1
(43) Pub. Date: Feb. 8, 2024

(54) TARGETING OF PERI-NECROTIC TUMOR CELLS

(71) Applicant: Fred Hutchinson Cancer Center, Seattle, WA (US)

(72) Inventors: Kevin Cheung, Mercer Island, WA (US); Ami Yamamoto, Seattle, WA (US); Brad Krajina, Shoreline, WA (US); Yin Huang, Bothell, WA (US)

(73) Assignee: Fred Hutchinson Cancer Center, Seattle, WA (US)

(21) Appl. No.: 18/365,110

(22) Filed: Aug. 3, 2023

Related U.S. Application Data
(60) Provisional application No. 63/394,929, filed on Aug. 3, 2022.

(51) Int. Cl. C07K 16/22; A61K 45/06; A61P 35/00; G01N 33/574

(52) U.S. Cl. CPC C07K 16/22; A61K 45/06; A61P 35/00; G01N 33/574; G01N 2800/52

(57) ABSTRACT

Provided herein are methods to reduce tumor cell proliferation in a cancer patient by administering an antibody that binds angiopoietin-like 7, wherein administering the angiopoietin-like 7 antibody can reduce necrosis formation, metastasis formation, and circulating tumor cells.

### CROSS-REFERENCE(S) TO RELATED APPLICATION(S)

This is a U.S. non-provisional application which claims the benefit of U.S. Provisional Application No. 63/394,929, filed Aug. 3, 2022, the disclosure of which is hereby incorporated by reference in its entirety for all purposes.

### STATEMENT OF GOVERNMENT LICENSE RIGHTS

This invention was made with government support under W81XWH-18-1-0098 awarded by the Medical Research and Development Command, and GM007270, CA080416, and CA260932 awarded by the National Institutes of Health. The government has certain rights in the invention.

### BACKGROUND

The development of metastasis is the pivotal determinant of long-term survival in most human cancers. A key step in this process is the dissemination of tumor cells into the systemic circulation. A long-standing question is where tumor cells disseminate from. A major focal point of investigation is the tumor-stromal border where cancer cells are directly observed to invade singly or collectively into surrounding tissues and disseminate into local blood vessels. Aiding this process are local conditions in the tumor microenvironment, such as hypoxia, acidity, and nutrient deficiency, in partnership with immune cells, that promote tumor dissemination. Counterintuitively, many of these microenvironmental influences are most prevalent in the tumor core, in regions of disordered tissue and blood vessel microarchitecture, where nutrient and oxygen availability are most limited. One way to reconcile these competing observations is that tumor dissemination can also occur in the tumor core. Consistent with this, the tumor core harbors abnormal biomechanics, increased interstitial pressure, and vascular leakiness thought conducive to dissemination. Further, tumor cells in the interior are actively migratory and spatially coordinated and tumor cell intravasation was determined to occur almost exclusively in the tumor core in an avian model system. These observations suggest mechanisms for metastatic dissemination from the tumor core. However, owing to the spatial heterogeneity and cellular complexity of the tumor core ecosystem, the molecular factors regulating dissemination from within and the role of tumor cells, as active drivers or passive participants in this process, are not understood.

In the setting of extreme nutrient limitation and vascular compromise, tumor cells undergo necrotic cell death. Necrosis is a pervasive feature of many aggressive fast-growing tumors, associated with poor prognosis and markedly increased risk of metastasis. A challenge of between-tumor/between-patient association studies is that it is not possible to disentangle if necrosis is a regulator of metastasis or a by-product of other more aggressive features such as tumor grade or tumor subtype. In this regard, spatial analyses within regions of the same tumor are suggestive. Spatial analyses of blood vessel invasion in lung cancers, sarcomas, hepatocellular carcinoma, and breast cancers have observed that between 25 to 50% of all blood vessel invasion events occur intratumorally in the tumor core. Likewise, spatial and temporal multiregional sequencing of primary and metastatic renal cell carcinomas reveal that metastatic subclones preferentially originate from the tumor interior where necrosis and increased copy number alterations predominate. Tumor cells that are dead cannot themselves give rise to metastasis. However, these clinical observations indicate that tumor cells neighboring the most intense regions of necrosis make productive contributions to metastatic dissemination. How the necrotic core promotes metastatic dissemination remains unclear.

In view of the limitations of the present art, a need remains for an effective therapeutic to target pen-necrotic tumor cells to reduce metastatic dissemination. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with the forgoing, in one aspect, the disclosure provides for a method of inhibiting necrotic core formation in a tumor to reduce the risk of metastatic dissemination from the tumor core, the method can comprise: (a) determining a level of circulating tumor cells (CTCs) in a biological sample from a cancer patient; (b) comparing the level of CTCs in the biological sample to a CTC reference level, if the level of CTCs in the biological sample is not greater than the CTC reference level, repeat steps (a) and (b) until the level of CTCs from the biological sample is greater than the CTC reference level; (c) identifying the cancer patient having an increased risk of metastatic dissemination if the level of CTCs from the biological sample is greater than the CTC reference level; and (d) administering an Angptl7 inhibitor to the cancer patient having an increased level of CTCs from the biological sample compared to the CTC reference level, wherein the Angptl7 inhibitor would reduce necrotic core formation and prevent metastatic dissemination.

In some embodiments, the Angptl7 inhibitor is an Angptl7 antibody.

In some embodiments, the sample is a blood sample.

In some embodiments, the Angplt7 antibody is administered intravenously.

In some embodiments, the Angptl7 antibody is administered intravenously for 60 minutes once every 4 weeks at a dose of 15 mg/kg.

In some embodiments, the method further comprises administering a second therapeutic agent, wherein the second therapeutic agent is an anti-cancer therapeutic agent.

In another aspect, the disclosure provides for a method of increasing survival of a cancer patient, the method can comprise administering a therapeutically effective amount of a composition to the cancer patient, the composition comprising a therapeutically effective dose of an angiopoietin-like 7 (Angptl7) inhibitor and a pharmaceutically acceptable carrier, wherein the Angptl7 inhibitor and the pharmaceutically acceptable carrier are administered as a unit dosage. In some embodiments, the Angptl7 inhibitor is an Angptl7 antibody. In some embodiments, the composition is administered intravenously. In some embodiments, the composition is administered intravenously for 60 minutes once every 4 weeks at a dose of 15 mg/kg. In some embodiments, the composition further comprises a second therapeutic agent, wherein the second therapeutic agent is an anti-cancer therapeutic agent. In some embodiments, the composition reduces expression and/or function of Angptl7 in a tumor. In some embodiments, the composition results in preventing at least one indication of aggressive tumor cell behavior. In some embodiments, the at least one indication of aggressive tumor cell behavior comprises necrosis formation in the tumor core. In some embodiments, the at least one indication of aggressive tumor cell behavior comprises metastasis formation. In some embodiments, the at least one indication of aggressive tumor cell behavior comprises an increase in circulating tumor cells (CTCs).

In another aspect, the disclosure provides for a method for reducing tumor cell proliferation in a cancer patient, the method can comprise the steps of: (a) collecting a sample from a cancer patient; (b) detecting an angiopoietin-like 7 (Angptl7) expression level in the sample collected from the cancer patient; (c) comparing the Angptl7 expression level in the sample collected from the cancer patient to an Angptl7 reference level; and (d) administering to the cancer patient a therapeutically effective dose of an Angptl7 inhibitor, if the Angptl7 expression level in the sample collected from the cancer patient is greater compared to the Angptl7 reference level, wherein inhibiting Angptl7 expression and/or function reduces at least one indication of tumor cell proliferation. In some embodiments, the Angptl7 inhibitor is an Angptl7 antibody. In some embodiments, the at least one indication of tumor cell proliferation comprises necrosis formation in the tumor core, metastasis formation, or an increase in circulating tumor cells (CTCs). In some embodiments, the method further comprises administering a second therapeutic agent, wherein the second therapeutic agent is an anti-cancer therapeutic agent.

### DESCRIPTION OF THE DRAWINGS

The patent or application file contains at least one drawing executed in color. Copies of this patent or patent application publication with color drawing(s) will be provided by the Office upon request and payment of the necessary fee. The foregoing aspects and many of the attendant advantages of this invention will become more readily appreciated as the same become better understood by reference to the following detailed description, when taken in conjunction with the accompanying drawings, wherein:

FIGS. 1A through 1J. CTC transition is temporally associated with tumor necrosis, dilated vessels, and perinecrotic intravascular emboli. FIG. 1A, Experimental schema. 4T1 mouse mammary tumor cells labeled with cytoplasmic GFP (4T1-GFP) were orthotopically transplanted into a single #4 mammary fat pad of SRG rats. Blood and tissues were harvested at day 13 (n=7), 17 (n=7), 22 (n=8), and 27 (n=8) posttransplantation. FIGS. 1B and 1C, Single CTC and CTC clusters per animal. Box plots shown with mean values labeled. FIG. 1D, Representative hematoxylin and eosin (H&E) stains of day 13 to 27 rat tumors. (Left) Necrotic region indicated with yellow border. (Right, Insets) of necrotic and nonnecrotic regions from a day 27 tumor. FIGS. 1E and 1F, Total necrotic area and total viable area were determined by H&E. Mean±SD. FIG. 1G, Representative day 13 to day 27 rat tumors stained for VE-cadherin (DAB) and counterstained with hematoxylin. (Left) low power views. Necrotic regions shown in turquoise, dilated vessels in red, and tumor border in magenta. (Right) example of a dilated and nondilated vessel. Dv=dilated vessel. Nz=necrotic zone. FIG. 1H, Number of VE-cadherin+ dilated blood vessels. Mean±SD. FIGS. 1I and 1J, Representative immunofluorescent images of GFP+ tumor emboli inside VE-cadherin+ blood vessel from thin (10 μm) and thick (30 μm) tumor sections. FIG. 1I (Left) low power image of perinecrotic zone. Insets showing high magnification of tumor cell cluster (tcc) within a dilated vessel (dv) FIG. 1I (Middle and Right). FIG. 1J, Confocal images from z-stack showing tumor cell cluster (tcc) within dilated vessel (dv). Xy, xz, and yz cross-sections shown. Magenta: VE-cadherin; Red: 594 conjugated lectin; Blue: DAPI; Green: tumor cells expressing GFP (membrane GFP in I & cytoplasmic GFP in J. nz=necrosis. P-values for B, C, E, F, and H determined by one-way ANOVA.

FIGS. 2A through 2E. Angptl7 is a tumor-derived, necrotic core-enriched transcript localized to the perinecrotic region of breast tumor. FIG. 2A, Experimental schema. 4T1-GFP tumor cells were orthotopically transplanted into a single fat pad of SRG rats and collected between day 27 and day 30 (n=5 animals). The harvested tumors were cut in half, and macrodissected to separate the necrotic core region from the nonnecrotic rim of the primary tumors. RNA was extracted and subjected to next-generation sequencing. Sequences were aligned to concatenated rat-mouse combined genome, and mouse and rat genes were deconvoluted from each other. FIG. 2B, Mouse orthologs were identified for all rat genes and the relative RNA abundance per gene between tumor and host compartments was determined. The plot shows all genes with both differential enrichment for core:rim and for tumor:host with FDR≤0.01. FIG. 2C, Relative RNA abundance of Angptl7 based on RNA-seq. Average expression and q-value (FDR) from multiple comparisons indicated on the graph. FIG. 2D, ELISA quantification of Angptl7 protein abundance of the necrotic core and nonnecrotic rim regions of 4T1 tumors. Student t test. Mean±SD. FIG. 2E, Representative image of Angptl7 RNA in situ hybridization (ISH) of day 27 4T1 tumors. (Left) raw Angptl7 RNA ISH (brown). (Right) Detection by QuPath. Red: detection of Angptl7+ cells, turquoise: necrotic region, pink: tumor border. *P<0.05, **P<0.01, ***P<0.001, and ****P<0.0001.

FIGS. 3A through 3G. Suppression of Angptl7 normalizes tumor necrosis. FIG. 3A, Experimental schema. 4T1 tumor cells labeled with membrane GFP and transduced with Angplt7 shRNA or nontargeting control were orthotopically transplanted into a single mammary fat pad of SRG rats. shRNA contained an mCherry tag, so cells expressing shRNA have cytoplasmic mCherry label. Blood, tumor, and lungs were collected. Nontargeting control (n=11), Angptl7 knockdown KD1 (n=7), Angptl7 KD2 (n=6), Angptl7 KD3 (n=7). FIG. 3B, In vivo knockdown confirmation by qPCR based on tumor core. FIG. 3C, ELISA confirmation by in vivo knockdown. Lysates were made from tumor necrotic core from Angptl7 knockdown and nontargeting control tumor cell transplantation experiments. FIG. 3D, Representative hematoxylin and eosin (H&E) staining of primary tumors for Angptl7 knock down tumors and nontargeting control. Yellow borders indicate the necrotic regions. FIGS. 3E and 3F, Total necrotic area and total viable area based on H&E staining of Angptl7 KD and nontargeting control tumor. FIG. 3G, Tumor weight of Angptl7 KD and nontargeting control tumor. All graphs shown display mean±SD. Mean values shown on graphs. P-values for B and C determined by Mann-Whitney test; for E-G determined by ANOVA. *P<0.05, **P<0.01, ***P<0.001, and ****P<0.0001.

FIGS. 4A through 4E. Suppression of Angptl7 reduces CTC abundance and distant lung metastases. FIG. 4A, Representative images of single CTCs and CTC clusters from nontargeting control. Cells are mCherry-positive if they express shRNAs. 4T1 cells are labeled by membrane GFP. DAPI marks nuclei. FIGS. 4B and 4C, mCherry-positive single CTC and CTC cluster abundance in Angptl7 knockdown and nontargeting control. FIG. 4D, Representative images of lung metastases from Angptl7 knockdown or nontargeting control transplantation (4T1 cells). Cells expressing the shRNAs express mCherry. FIG. 4E, mCherry-positive lung metastasis count for Angptl7 knockdown and nontargeting control. Mean±SD. All graphs shown display mean±SD. Mean values shown on graphs. P-values for B, C, and E determined by ANOVA. *P<0.05, **P<0.01, ***P<0.001, and ****P<0.0001.

FIGS. 5A through 5F. Angptl7 regulates blood vessel morphology and vascular permeability. FIG. 5A, Representative images of dilated VE-cadherin+ blood vessels Angptl7 knockdown and nontargeting control tumors. Immunohistochemistry for VE-cadherin (DAB) (brown) counterstained with hematoxylin. Red: dilated vessels, turquoise: necrotic region, pink: tumor border. (Insets): vessel morphology in nontargeting control and Angptl7 knockdown. FIG. 5B, Number of total VE-cadherin+ blood vessels in the Angptl7 knockdown and nontargeting control tumors. Mean±SD. FIG. 5C, Number of dilated VE-cadherin+ blood vessels in the Angptl7 knockdown and nontargeting control tumors. Mean±SD. FIGS. 5D and 5E, Human endothelial cell (HUVEC) vascular permeability in response to ANGPTL7 recombinant protein (rhANGPTL7). Vascular permeability was measured as normalized impedance over time. n=4 biological replicates. Shown is mean temporal response (D) and normalized cell index at 1 h (E). FIG. 5F, Metascape analysis of gene enrichment. HUVEC cells were treated with rhANGPTL7 for 24 h. RNA-seq identified 741 up-regulated genes and 500 genes down-regulated with rhANGPTL7 treatment with P-value cutoff ≤0.01. Enrichments reported as −log Q-values. All P-values determined by one-way ANOVA. *P<0.05, **P <0.01, ***P<0.001, and ****P<0.0001.

FIGS. 6A through 6G. ANGPTL7 is expressed in high necrosis human triple-negative breast cancers and necrosis markers correlate with CTC dissemination in breast cancer patients. FIG. 6A, ANGPTL7 RNA ISH performed on tumor sections from human breast PDX tumors transplanted into NSG mice. All tumor models were invasive ductal carcinomas and confirmed triple-negative for ER, PR, and HER2 on PDX tumor sections. Qupath detections shown. Turquoise: necrotic region, red: ANGPTL7-positive detections, pink: tumor border. (Insets) show raw ANGPTL7 RNA ISH in red. (Right) Bar graph comparing tumors with low necrosis (five sections from four models) and high necrosis (two sections from two models). High necrosis defined as >15% necrosis by area. P-value determined by unpaired t test. FIG. 6B, Representative single CTC and CTC cluster from clinical vignette in FIG. 12. EpCAM/pan-CK, epithelial cells; CD45, immune cells; SYTOX Orange, nucleic acid stain. FIG. 6C, Overall survival according to CTC enumeration at baseline. Median overall survival reported in parentheses. NR=not reached. P-value determined by Mantel-Cox log-rank test. FIG. 6D, Overall survival according to presence or absence of CTC-clusters. Landmark analysis from 2nd blood draw was performed. Median overall survival reported in parentheses. P-value determined by Mantel-Cox log-rank test. FIG. 6E, Waterfall plot showing changes in CTC abundance between time points, sorted by magnitude. FIG. 6F, Tandem-mass tag mass spectrometry of high vs. low-CTC samples. Volcano plot shows the 46 plasma proteins enriched in 3 high-CTC samples compared with 13 low CTC samples. Proteins with Q-values less than 10%. False discovery rate determined by two-stage setup method by Benjamini, Krieger, and Yekutieli. FIG. 6G, Metascape analysis of CTC-associated proteins reported by −log Q-value. (Right) Proteins with fold-enrichment that make up core enrichments for Cori cycle and 20S proteasome. *P<0.05, **P<0.01, ***P<0.001, and ****P<0.0001.

FIGS. 7A through 7J. Orthotopic transplantation into rats produce 3× larger tumors, 10× more CTCs, and 4× more lung metastases than into mice. FIG. 7A, Experimental schema. 4T1 mouse mammary tumor cells labeled with membrane GFP (4T1-GFP) were orthotopically transplanted into a single #4 mammary fat pad of SRG rats (n=6) or NSG mice (n=24). Animals were sacrificed at 24 days post-transplantation. FIGS. 7B and 7C, Final tumor weight and estimated final tumor volume per animal. FIG. 7D, Blood volume collected per animal. FIG. 7E, Representative micrographs of single CTCs and CTCs clusters from 4T1-GFP transplanted SRG rats. GFP denoted in green. DAPI in blue. FIGS. 7F and 7G, Single CTC and CTC cluster abundance per animal. Individual blood samples from 8 mice were pooled into one tube for a total of n=3 samples. Events per individual animal are reported. FIG. 7H, Percentage of CTC events that were single CTCs or CTC clusters. Mean±SD. FIG. 7I, Representative stereomicroscope images of lung metastases from NSG mice and SRG rats. FIG. 7J, The number of lung metastases determined by stereomicroscopy in transplanted SRG rats and NSG mice. Mean values shown on graphs. All P-values determined by Welch's t-test.

FIGS. 8A through 8K. Additional morphometric parameters and their correlation with low to high CTC transition. FIGS. 8A and 8B, Estimated tumor volume and final tumor weight determined from animals collected as in FIG. 1A. Mean±SD. FIGS. 8C and 8D, Single CTC and CTC clusters per animal plotted on log scale. Mean ±SD. FIG. 8E, Representative H&E images of the invasive tumor border. FIG. 8F, Representative images of TTC stained tumors. Red areas are viable tissue while white areas are necrotic tissue. Unstained control is provided. Yellow borders indicate necrotic region. FIGS. 8G and 8H, Total area of necrosis and viable area determined from the TTC assay. n=4 animals per time point; 2 to 3 slices per tumor. Mean±SD. FIG. 8I, Lung metastases per animal. Mean±SD. FIGS. 8J and 8K, Correlation between lung metastases and necrotic area (J) and lung metastases and tumor size (K). Pearson R. Linear trend lines shown with 95% confidence intervals. P-values for A-D and I determined by one-way ANOVA. P-Values for G-H determined by Welch's t-test.

FIGS. 9A through 9J. Additional information on characteristics of blood vessels. FIG. 9A, Dilated blood vessel spatial localization. Distance of VE-cadherin-positive dilated vessels to necrosis or tumor border was measured then normalized to a spatial proximity score of 0 to 1, where 0 means a spot is on the tumor border, and 1 means a spot is in a necrotic region. Spatial localization of all cells were plotted as a control. P-value determined by Kruskal-wallis test. FIG. 9B, Dilated VE-cadherin vessel density per mm2 viable tumor area determined per animal. Mean±SD. FIG. 9C, Total VE-cadherin abundance determined per animal. Mean±SD. FIG. 9D, Total VE-cadherin vessel density per mm2 viable tumor area determined per animal. Mean±SD. FIGS. 9D-9H, Correlation between lung metastasis or CTC events with dilated vessels (E) and total vessels (F). Correlation between CTC events and dilated vessels (G) and total vessels (H). Pearson R. Linear trend lines shown with 95% confidence intervals. FIG. 9I, Representative immunofluorescence images of dilated and non-dilated blood vessels in 4T1 tumors perfused with lectin-594 and stained for VE-cadherin. FIG. 9J, Percent lectin-positive VE-cadherin-positive blood vessels in 4T1 transplant tumors. Mean±SD. P-values for B-D determined by one-way ANOVA; J by paired t-test.

FIGS. 10A through 10H. Additional information on tumor core transcriptional profiling. FIG. 10A, Representative images of necrotic cores of primary tumors used for bulk RNA-seq. FIG. 10B, Percent of transcripts which mapped to the mouse or rat genome. 4T1 cell pellets served as control for mouse-only samples, and rat mammary tissue was used as rat-only samples. Average percentage and standard deviations are shown in the table. FIG. 10C, Top tumor or host-derived gene sets enriched in the tumor core and rim, based on Metascape analysis. Q-value (FDR—False discovery rate)≤0.01 was used as the cut-off for the gene list. R=reactome, W=WikiPathways, G=GO ontology, K=KEGG C=Canonical Pathways. FIG. 10D, Relative RNA abundance of tumor or host-derived Camkld expression in the tumor core or rim. Camkld is the #1 most enriched tumor-derived gene in the tumor core. Camkld is also expressed by the host and is not tumor specific. Average expression and q-value (FDR) from multiple comparisons indicated on the graph. FIG. 10E, Angptl7 expression in 4T1 tumor core, 4T1 tumor rim, and 2D culture 4T1 cells based on qPCR. Mann-Whitney. Average values shown. FIG. 10F, Western blot of necrotic core and non-necrotic rim regions of 4T1 tumors to assess protein-level differential expression of Angptl7. Angptl7 is highly expressed in necrotic region of the tumor but not the non-necrotic region. GAPDH was used as loading control. 4T1 cells in vitro do not express Angptl7 and was used as the negative control. N=3 rat tumors. FIG. 10G, Quantification of protein-level expression of Angptl7 from 10F. P-value determined by paired t-test. FIG. 10H, Angptl7 spatial localization. Distance of Angptl7+ spots to either necrosis or tumor border was measured then normalized to a spatial proximity score of 0 to 1, where 0 means a spot is on the tumor border, and 1 means a spot is in a necrotic region. Spatial localization of all cells are plotted as a control. P-value determined by Kruskal-wallis test.

FIGS. 11A through 11E. Angptl7 expression increase with day post-transplantation, correlates with necrotic area, lung metastases, and dilated vessels. FIG. 11A, Angptl7 detections from day 13 to day 27 post-transplantation based on RNA ISH. P values determined by one-way ANOVA. FIGS. 11B-11E, Correlation between Angptl7 detections per tumor and necrotic area, lung metastases, dilated blood vessels, or CTC events. Pearson R. Linear trend lines shown with 95% confidence intervals.

FIGS. 12A through 12F. Additional information on in vivo effects of Angptl7 suppression. FIG. 12A, Quantification of ELISA of necrotic core and whole 4T1 tumors for Angptl7. Angptl7 is highly expressed in necrotic region of the tumor. Non-targeting control (n=7), KD1 (n=7), KD2 (n=3), and KD3 (n=4). Paired t-test. FIG. 12B, Estimated final tumor volume of non-targeting and Angptl7 KD tumors. FIG. 12C, Representative images of single CTCs and CTC clusters from Angptl7 knockdown or nontargeting control transplantation into SRG rats. Cells are mCherry-positive if they express shRNAs. 4T1 cells are labeled by membrane GFP. DAPI marks nuclei. FIGS. 12D and 12E, GFP-positive single CTC and CTC cluster abundance in Angptl7 knockdown and nontargeting control. FIG. 12F, GFP+ lung metastases. All graphs reported as mean±SD and p-values determined by one-way ANOVA.

FIGS. 13A through 13C. Additional information on in vivo effect of Angptl7 suppression on gene expression. FIG. 12A, Gene sets were generated composed of tumor-derived and host-derived core and rim genes with fold change ≥2 and FDR≤0.001. The relative enrichment of these 4 gene sets was determined for tumor core or rim from Angptl7 knockdown and non-targeting control. FIG. 13B, Genes within tumor-derived and host-derived necrotic core gene sets with FDR≤0.01 were stratified by t-statistic and divided into genes up or down in Angptl7-kd conditions. FIG. 13C, Metascape analysis of tumor and host-derived tumor core gene program stratified by their change in expression with Angptl7 suppression. Gene set enrichment reported as log Q-value.

FIGS. 14A through 14G. Additional information on in vivo effect of Angptl7 suppression on blood vessels. FIG. 14A, Total VE-cad+ blood vessel density per mm2 viable tumor area per animal for Angptl7 KD and non-targeting control tumors. FIG. 14B, Dilated VE-cad+ blood vessel density per mm2 viable tumor area per animal for Angptl7 KD and non-targeting control tumors. FIG. 14C, Representative imaged of alpha smooth actin immunohistochemistry. VE-cadherin in vector red (pink), alpha smooth actin in NOVA Red (brown), counterstained with hematoxylin (purple). FIG. 14D, Alpha smooth actin-positive (aSMA) coverage on VE-cadherin positive dilated vessels in non-targeting control and Angptl7 KD tumors. FIG. 14E, Number of VE-cadherin positive blood vessels with no aSMA coverage in non-targeting control and Angptl7 KD tumors. FIG. 14F, Number of VE-cadherin positive blood vessels with aSMA coverage in non-targeting control and Angptl7 KD tumors. All graphs shown as mean±SD. P-values determined by one-way ANOVA.

FIGS. 15A through 15D. In vivo effect of Angptl7 suppression on lymphatic vessels. FIG. 15A, Representative images podoplanin+ lymphatic vessels in Angptl7 knockdown and nontargeting control tumors. Immunohistochemistry for podoplanin (DAB) (brown) counterstained with hematoxylin. Turquoise: necrotic region, pink: tumor border. Navy border inset is an example of the tumor edge. Magenta border inset is an example of a peri-necrotic region. FIG. 15B, Number of podoplanin+ lymphatic vessels in the Angptl7 knockdown and non-targeting control tumors. Mean±SD. One-way ANOVA. FIG. 15C, Spatial enrichment of non-targeting control tumors comparing all cells (control distribution) with all podoplanin+ lymphatic vessels. P-value determined by Kruskal-wallis test. FIG. 15D, Spatial enrichment of podoplanin+ lymphatic vessels in Angptl7 KD and non-targeting control tumors. Mean±SD. One-way ANOVA.

FIGS. 16A through 16F. Supplementary information on correlation of CTC and necrosis in breast cancer patients with metastatic disease. FIG. 16A, Clinical vignette of patient with metastatic breast cancer and acute CTC elevation. This was a patient with de novo stage IV ER+PR+HER2- metastatic breast cancer involving the right breast, lymph node, bones, liver and lung. Despite initial response to endocrine therapy in combination with the CDK4/6 inhibitor palbociclib, the patient progressed and was switched to capecitabine monotherapy. Over a 30-week period, the patient demonstrated increasing blood levels of tumor marker CA15-3, indicating progressive resistance to therapy. At week 4, an initial CTC determination yielded a CTC count of 8 per 7.5 mL and no CTC clusters. At week 24, a repeat CTC determination showed a CTC count of 1456 per 7.5 mL and 26 CTC clusters per 7.5 mL. Strikingly, lactate dehydrogenase (LDH), a marker of tissue necrosis, also increased over the same period: 309 at week 4 to 963 at week 24 and 3727 U/L at week 26, during inpatient admission for workup of abdominal pain, liver injury, and tumor lysis. Treatment course: (1) capecitabine, (2) in patient admission for liver injury and tumor lysis, (3) death. CA15.3, cancer antigen 15-3; LDH, serum lactate dehydrogenase. FIG. 16B, Dual energy scanning contrast tomography. Baseline scan obtained 1 month prior to first draw (CTC=8 per 7.5 mL). Follow-up scan obtained 1 week after second draw (CTC=1456 per 7.5 mL) during in hospital admission. Upper panels: contrast enhanced CT. Lower panels: iodine distribution. The right lobe liver metastasis (yellow outline) measured 3.1 cm at baseline with a maximum iodine concentration of 38 μg/cm3 and an average of 19.2 μg/cm3 which grew to 4.7 cm on follow up 3 months later and the maximum iodine concentration decreased to 32 μg/cm3 and the average to 14.2 μg/cm3. Lower iodine concentration is indicative of decreased perfusion and increased necrosis. FIG. 16C, Tandem-mass tag mass spectrometry of late versus early time points for low-to-high CTC transitions (n=3 patient sample pairs). Volcano plot shows the plasma proteins enriched in high CTC samples with P-values less than 0.1. Metascape analysis of proteins reported by −log Q-value. Listed are proteins with 2-or-more fold-enrichment in high CTC samples. Necrosis-associated proteins highlighted in blue. FIG. 16D, Change in LDH between time points according to change in CTC abundance between time points. P-values determined by one-way ANOVA comparing against 10+ or more CTCs. FIG. 16E, Serum LDH in relation to CTCs per sample. N=39 patients, 93 blood samples. P-values determined by one-way ANOVA comparing against 20+ CTCs. FIG. 16F, Serum LDH in relation to presence or absence of CTC clusters. N=39 patients, 93 blood samples. P-values determined by Mann-Whitney test.

FIG. 17. Aggressive tumors undergo necrosis (N) promoting drug resistance, tumor evolution, and metastasis. The development of necrosis is dependent on a secreted protein Angptl7 (A7). As illustrated, an A7 therapeutic antibody can suppress tumor growth and metastasis.

FIGS. 18A and 18B. A7 suppression remodels the tumor microenvironment and prevents tumor necrosis, growth, and metastasis. 4T1 tumor cells were transduced with non-targeting control (NTC-kd) or Angptl7-shRNA (A7-kd) and transplanted into NSG mice. NSG mice transplanted with NTC-kd or A7-kd tumor cells were treated with cremaphor vehicle or 15 mg/kg i.p. paclitaxel (P) and then harvested for tumor (FIG. 18A) and lung metastases (FIG. 18B).

FIGS. 19A and 19B. ANGPTL7 is necessary and sufficient for maximal tumor cell proliferation. 4T1 cell lines and derivatives were plated in 2D and assayed for cell impedence kinetics using the xCelligence platform. Cell index is determined relative to baseline measurement at t=0 and is a proxy for cell number. FIG. 19A, Shown are 4T1 cells stably transduced with a control mCherry vector or human ANGPTL7 overexpression lentiviral vector and analyzed for cell impedence kinetics. Overexpression of ANGPTL7 increases maximal tumor cell proliferation in growth limited conditions. FIG. 19B, Control-kd tumor cells or ANGPTL7-kd tumor cells were orthotopically transplanted into SRG rats and then isolated at maximal tumor size and expanded ex-vivo. Control-kd tumor isolates and ANGPTL7-kd tumor isolates were analyzed for cell impedence kinetics. Together A and B show that ANGPTL7—a factor expressed in pen-necrotic tumor cells—is necessary and sufficient for maximal tumor cell proliferation.

FIG. 20. Specific binding of ANGPTL7 antibody to human ANGPTL7 full length protein. In this experiment, 4T1 cells were stably transduced with constitutive expression vectors encoding either control mCherry protein (mCH) or full-length human ANGPTL7 (hA7). Confluent 4T1-mCH or 4T1-hA7 cell lines were subsequently detected using a cell-based ELISA, blocked, detected with polyclonal antibody directed recognizing human, mouse, and rat Angptl7 (PA536575) and subsequently reported with HRP secondary antibody and TMB colorimetric detection. These data show that antibody can bind specifically to native human ANGPTL7 in cell-based assays.

### DETAILED DESCRIPTION

Necrosis in the tumor interior is a common feature of aggressive cancers that is associated with poor clinical prognosis and the development of metastasis. How the necrotic core promotes metastasis remains unclear. As described further in the Examples section, the emergence of necrosis inside the tumor is correlated temporally with increased tumor dissemination in a rat breast cancer model and in human breast cancer patients. By performing spatially focused transcriptional profiling, angiopoietin-like 7 (Angptl7) was identified as a tumor-specific factor localized to the perinecrotic zone. Functional studies showed that Angptl7 loss normalizes central necrosis, perinecrotic dilated vessels, metastasis, and reduces circulating tumor cell counts to nearly zero. Mechanistically, Angptl7 promotes vascular permeability and supports vascular remodeling in the perinecrotic zone. Taken together, these findings show that breast tumors actively produce factors controlling central necrosis formation and metastatic dissemination from the tumor core.

Definitions

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa unless the content clearly dictates otherwise. In the event that any description of a term set forth conflicts with any document incorporated herein by reference, the description of the term set forth below shall control.

The terms “a”, “an”, and “the”, as used herein, include plural references unless the context clearly dictates otherwise.

The term “about”, as used herein, in reference to a number or range of numbers, is understood to mean the stated number and numbers +/−10% thereof, or 10% below the lower listed limit and 10% above the higher listed limit for the values listed for a range.

The term “between”, as used in a phrase as such “between A and B” or “between A-B” refers to a range including both A and B.

The terms “or” and “and/or”, as used herein, include any, and all, combinations of one or more of the associated listed items.

The terms “including”, “includes”, “included”, and other forms, as used herein, are not limiting.

The terms “comprise” and its grammatical equivalents, as used herein, specify the presence of stated features, integers, steps, operations, elements, and/or components, but do not preclude the presence or addition of one or more other features, integers, steps, operations, elements, components, and/or groups thereof.

The term “administer”, “administration”, or “administering”, as used herein refers to the act of injecting or otherwise physically delivering a substance (e.g., a pharmaceutical composition provided herein) to a subject, such as by oral, mucosal, topical, intradermal, parenteral, intravenous, intravitreal, intraarticular, subretinal, intramuscular, intrathecal delivery and/or any other method of physical delivery described herein or known in the art. The delivery can be systemic or to a specific tissue.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, and multispecific antibodies (e.g., bispecific antibodies so long as they exhibit the desired biological activity). A conventional antibody is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck, ed., 2d ed. 1995); and Kuby, Immunology (3d ed. 1997). An antibody can be human, humanized, chimeric and/or affinity matured, as well as an antibody from other species, for example, mouse and rabbit, etc. Antibodies also include, but are not limited to, synthetic antibodies, recombinantly produced antibodies, camelized antibodies or their humanized variants, and intrabodies. An antibody can be selected from any class of immunoglobulins, including IgM, IgG, IgD, IgA and IgE, and any isotype, including IgG1, IgG2, IgG3 and IgG4 (e.g., variants of IgG4 and IgG4 nullbody). An antibody can comprise kappa or lambda light chain constant sequences.

The term “antigen” refers to a molecule or a portion of a molecule capable of being bound by an antibody or an antigen-binding fragment thereof and additionally capable of being used in an animal to produce antibodies capable of binding to an epitope of that antigen. An antigen may be a polypeptide, carbohydrate, nucleic acid, lipid, hapten, or other naturally occurring or synthetic compound.

The term “binds” or “binding”, as used herein, refers to a covalent or non-covalent interaction between molecules (e.g., forming a complex by interactions). Exemplary non-covalent interactions include hydrogen bonds, ionic bonds, hydrophobic interactions, and/or van der Waals interactions. As used herein, the term “specifically binds” refers to binding of an antibody or an antigen binding fragment thereof to an antigen with a dissociation constant (K_{D})≤10⁻⁷ M. The term “K_{D}” is intended to refer to the dissociation equilibrium constant of a particular antibody-antigen interaction. The ratio of dissociation rate (k_{off}) to association rate (kₒₙ) of an antibody to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. Specific binding can be measured, for example, by determining binding of a molecule compared to binding of a control molecule, which generally is a molecule of similar structure that does not have binding activity.

The term “effective amount” or “therapeutically effective amount”, as used herein, refers to an amount of a therapeutic (e.g., Angptl7 inhibitor or a pharmaceutical composition provided herein) which is sufficient to treat, diagnose, prevent, delay the onset of, reduce and/or ameliorate the severity and/or duration of a given condition, disorder or disease and/or a symptom related thereto. The term also encompasses an amount necessary for the reduction, slowing, or amelioration of the advancement or progression of a given disease, reduction, slowing, or amelioration of the recurrence, development or onset of a given disease, and/or to improve or enhance the prophylactic or therapeutic effect(s) of another therapy or to serve as a bridge to another therapy.

The term “epitope”, as used herein, refers to a localized region of an antigen to which an antibody can bind. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. In some embodiments, an antibody binds to a group of amino acids regardless of whether they are folded in a natural three dimensional protein structure. In some embodiments, an antibody requires amino acid residues making up the epitope to exhibit a particular conformation (e.g., bend, twist, turn or fold) in order to recognize and bind the epitope.

The term “monoclonal antibody,” as used herein, refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen.

The term “polyclonal antibody” as used herein, refers to a collection of antibodies from different B cells that recognize multiple epitopes on the same antigen.

The term “pharmaceutically acceptable excipient, carrier or diluent”, as used herein, refers to any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by well-known conventional methods.

The term “pharmaceutical composition” or “therapeutic composition”, as used here, refers to a composition capable of being administered to a subject for the treatment of a particular disease or disorder.

The term “prevent”, as used herein, refers to a pharmaceutical or other intervention regimen for reducing the likelihood of the onset (or recurrence) of a disease, disorder, condition, or associated symptom(s). Preventing includes delaying, preventing, or eliminating the appearance of a disease or condition, delaying, or eliminating the onset of symptoms of a disease or condition, slowing, halting, or reversing the progression of a disease or condition, or any combination thereof.

The term “subject”, as used herein, refers to an “animal” and in particular a “mammal” such as a non-primate (e.g., mice, rats, bovines, horses, household cats, tigers and other large cats, dogs, pigs, rabbits, goats, deer, sheep, ferrets, gerbils, guinea pigs, hamsters, bats, and birds (e.g., chickens, turkeys, and ducks)) or a primate (e.g., monkeys, baboons, chimpanzees, and human). The term may be used interchangeably with the term “patient” or “individual”. In some embodiments, the subject is a mammal, e.g., a human, diagnosed with a disease or disorder provided herein. In some embodiments, the subject is a mammal, e.g., a human, at risk of developing a disease or disorder provided herein. As used herein, “cancer patient” refers to a subject with cancer.

The terms “treatment” and “treating”, as used herein, refer to a pharmaceutical or other intervention regimen for obtaining beneficial or desired results in the recipient. Treating may refer to eradication or amelioration of symptoms or of an underlying disorder being treated. Treating may also be achieved with the eradication or amelioration of one or more of the physiological symptoms associated with the underlying disorder such that an improvement is observed in the subject, notwithstanding that the subject may still be afflicted with the underlying disorder.

The term “sample,” “test sample,” “specimen,” “biological sample,” “sample from a subject,” or “subject sample” as used herein interchangeably, means a sample or isolate of blood, tissue, urine, serum, plasma, amniotic fluid, cerebrospinal fluid, placental cells or tissue, endothelial cells, leukocytes, or monocytes, can be used directly as obtained from a subject or can be pre-treated, such as by filtration, distillation, extraction, concentration, centrifugation, inactivation of interfering components, addition of reagents, to modify the character of the sample in some manner as discussed herein or otherwise as is known in the art.

The term “risk,” “risk assessment,” “risk classification,” “risk identification,” or “risk stratification” as used herein interchangeably, means an evaluation of factors including biomarkers, to predict the risk of occurrence of future events including disease onset or disease progression, so that treatment decisions regarding the subject may be made on a more informed basis. Additionally, as used herein, the term “risk” (e.g., risk of metastatic dissemination) and any grammatical variants listed above are used to indicate that when practicing the disclosed methods, the method will reduce the likelihood of the patient progressing to metastatic dissemination. For example, prior to practice the disclosed method, the cancer patient has no or just very low levels of metastatic dissemination from the tumor core. In the absence of practicing the disclosed method, the cancer patient with no or just very low levels of metastatic dissemination from the tumor core will eventually progress to elevated metastatic dissemination from the tumor core. By practicing the claimed invention while the cancer patient has no or just very low levels of metastatic dissemination from the tumor core, the cancer patient will not progress to elevated metastatic dissemination from the tumor core.

As used herein, the terms “reducing,” “inhibiting,” “suppressing” and any grammatical variants thereof refer to decreasing or alleviating a condition associated with a tumor expressing high levels of angiopoietin-like 7. In some embodiments, the condition associated with a tumor expressing high levels of angiopoietin-like 7 can include but are not limited to tumor necrosis, tumor cell proliferation, CTCs, or metastasis. In some embodiments, the condition associated with a tumor expressing high levels of angiopoietin-like 7 can be decreased or alleviated by at least 10%, 20%, 30%, 40%, 50%, 60%, 70%, 80%, 90%, 91%, 92%, 93%, 94%, 95%, 96%, 97%, 98%, 99%, or 100%.

As used herein, Angiopoietin-like 7 (Angptl7) is a protein that is encoded by the ANGPTL7 gene. Angptl7 is one of 8 angiopoietin-like proteins that comprise an N-terminal coiled-coil domain which mediates homo-oligomerization and a C-terminal fibrinogen domain. ANGPTLs are widely expressed in the liver, vasculature and hematopoietic systems, and serve important roles in inflammation, lipid metabolism, angiogenesis and extracellular matrix (ECM) formation. In some embodiments, “ANGPTL7” and “angiopoietin-like 7” are inclusive of all family members, mutants, alleles, fragments, species, coding and noncoding sequences, sense and antisense polynucleotide strands, etc. of the ANGPTL7 transcript.

As used herein, the term “increase survival” and any grammatical variants refer to prolonging the life span of a cancer patient by treating the underlying conditions (e.g., necrosis, metastatic dissemination) caused by the cancer or curing the cancer, all of which will increase the patient's life span. In some embodiments, an increase in life span can include but is not limited to an increase as measured in days, months, or years.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Therapeutic Methods and Application

In accordance with the forgoing, in one aspect, the disclosure provides for a method of reducing tumor cell proliferation in a cancer patient, the method can comprise the steps of: (a) collecting a sample from a cancer patient; (b) detecting an angiopoietin-like 7 (Angptl7) expression level in the sample collected from the cancer patient; (c) comparing the Angptl7 expression level in the sample collected from the cancer patient to an Angptl7 reference level; and (d) administering to the cancer patient a therapeutically effective dose of an Angptl7 inhibitor, if the Angptl7 expression level in the sample collected from the cancer patient is greater compared to the Angptl7 reference level, wherein inhibiting Angptl7 expression and/or function reduces at least one indication of tumor cell proliferation.

In another aspect, the disclosure provides for a method of inhibiting tumor necrosis in a cancer patient, the method can comprise the steps of: (a) collecting a sample from the cancer patient; (b) detecting an Angptl7 expression level in the sample collected from the cancer patient; (c) comparing the Angptl7 expression level in the sample collected from the cancer patient to an Angptl7 reference level; and (d) administering to the cancer patient a therapeutically effective dose of an Angptl7 inhibitor, if the Angptl7 expression level in the sample collected from the cancer patient is greater compared to the Angptl7 reference level, wherein inhibiting Angptl7 reduces tumor necrosis.

In another aspect, the disclosure provides for a method of suppressing metastasis in a cancer patient, the method can comprise the steps of: (a) collecting a sample from the cancer patient; (b) detecting angiopoietin-like 7 (Angptl7) expression level in the sample collected from the cancer patient; (c) comparing the Angptl7 expression level in the sample collected from the cancer patient to an Angptl7 reference level; and (d) administering to the cancer patient a therapeutically effective dose of an Angptl7 inhibitor, if the Angptl7 expression level in the sample collected from the cancer patient is greater compared to the Angptl7 reference level, wherein inhibiting Angptl7 suppresses metastasis.

In another aspect, the disclosure provides a method for reducing circulating tumor cells (CTCs) in a cancer patient, the method comprising the steps of: (a) collecting a sample from the cancer patient; (b) detecting angiopoietin-like 7 (Angptl7) expression level in the sample collected from the cancer patient; (c) comparing the Angptl7 expression level in the sample collected from the cancer patient to an Angptl7 reference level; and (d) administering to the cancer patient a therapeutically effective dose of an Angptl7 inhibitor, if the Angptl7 expression level in the sample collected from the cancer patient is greater compared to the Angptl7 reference level, wherein inhibiting Angptl7 reduces CTCs.

In another aspect, the disclosure provides a method of inhibiting necrotic core formation in a tumor to reduce the risk of metastatic dissemination from the tumor core, the method can comprise: (a) determining a level of circulating tumor cells (CTCs) in a biological sample from a cancer patient; (b) comparing the level of CTCs in the biological sample to a CTC reference level, if the level of CTCs in the biological sample is not greater than the CTC reference level, repeat steps (a) and (b) until the level of CTCs from the biological sample is greater than the CTC reference level; (c) identifying the cancer patient having an increased risk of metastatic dissemination if the CTC level from the biological sample is greater than the CTC reference level; and (d) administering an Angptl7 inhibitor to the cancer patient having an increased level of CTCs compared to the CTC reference level, wherein the Angptl7 inhibitor would reduce necrotic core formation and prevent metastatic dissemination.

In another aspect, the disclosure provides for a method of increasing survival of a cancer patient, the method can comprise administering a therapeutically effective amount of a composition to the cancer patient, the composition comprising an angiopoietin-like 7

(Angptl7) inhibitor and a pharmaceutically acceptable carrier, wherein the Angptl7 inhibitor and the pharmaceutically acceptable carrier are administered as a unit dosage.

In another aspect, the disclosure provides a method of treating a necrosis-associated disease or condition, the method can comprise administering a therapeutically effective amount of a composition to a subject in need thereof, the composition comprising an angiopoietin-like 7 (Angptl7) inhibitor and a pharmaceutically acceptable carrier, wherein the Angptl7 inhibitor and the pharmaceutically acceptable carrier are administered as a unit dosage.

In some embodiments, the cancer patient can be a neonate, a juvenile, or an adult. In some embodiments, the cancer can be but is not limited to breast cancer, colon cancer, lung cancer, prostate cancer, testicular cancer, brain cancer, skin cancer, rectal cancer, gastric cancer, esophageal cancer, sarcomas, tracheal cancer, head and neck cancer, pancreatic cancer, liver cancer, ovarian cancer, lymphoid cancer, cervical cancer, vulvar cancer, melanoma, mesothelioma, renal cancer, bladder cancer, thyroid cancer, bone cancer, carcinoma, sarcoma, or soft tissue cancer.

In some embodiments, the sample can be a fluid sample. In some embodiments, the sample can be a tissue sample. In some embodiments, the sample can be any tissue sample taken or derived from the subject. In some embodiments, any cell type, tissue, or bodily fluid can be utilized to obtain a sample. In some embodiments, such cell types, tissues, and fluids can include sections of tissues such as biopsy and autopsy samples, frozen sections taken for histological purposes, blood (such as whole blood), plasma, serum, sputum, stool, tears, mucus, saliva, hair, skin, red blood cells, platelets, interstitial fluid, ocular lens fluid, cerebral spinal fluid, sweat, nasal fluid, synovial fluid, menses, amniotic fluid, or semen, etc. In some embodiments, the tissue can be from a tumor biopsy. In some embodiments, the biopsy can be an excisional biopsy, an incisional biopsy, or a liquid biopsy. In some embodiments, the biopsy can remove the entire tumor. In some embodiments, the biopsy can remove cells from the tumor core. In some embodiments, the biopsy can remove cells from the tumor periphery. In some embodiments, the sample can comprise a previously isolated fluid sample or a previously isolated tissue sample (i.e., isolated by another person at another time for another purpose). In some embodiments, the sample is a blood sample.

In some embodiments, the methods described herein include measuring the expression level of Angptl7. In some embodiments, the methods described herein include measuring the expression level of Angptl7 locally (e.g., in the tumor). In some embodiments, the methods described herein include measuring the expression level of Angptl7 systemically.

In some embodiments, the methods described herein include measuring the functional activity of Angptl7. In some embodiments, the methods described herein include measuring the functional activity of Angptl7 locally (e.g., in the tumor). In some embodiments, the methods described herein include measuring the functional activity of Angptl7 systemically.

In some embodiments, the functional activity of Angptl7 can be measured using any assay well-known to one of ordinary skill in the art.

In some embodiments, detecting the expression level of Angptl7 can be performed by an immunoassay, a colorimetric assay, PCR, or a fluorescence assay. In some embodiments, the assay can measure Angptl7 protein. In some embodiments, the Angptl7 protein measurement can be an Angptl7 protein level. In some embodiments, the Angptl7 protein level can be indicated as a mass or percentage of Angptl7 protein per sample weight. In some embodiments, the Angptl7 protein level can be indicated as a mass or percentage of Angptl7 protein per sample volume. In some embodiments, the Angptl7 protein level can be indicated as a mass or percentage of Angptl7 protein per total protein within the sample. In some embodiments, the Angptl7 protein measurement can be a baseline circulating Angptl7 protein measurement. In some embodiments, the Angptl7 protein measurement can be obtained by an assay such as an immunoassay, a colorimetric assay, or a fluorescence assay.

In some embodiments, the assay can measure Angptl7 mRNA. In some embodiments, the Angptl7 mRNA measurement comprises an Angptl7 mRNA level. In some embodiments, the Angptl7 mRNA level can be a mass or percentage of Angptl7 mRNA per sample weight. In some embodiments, the Angptl7 mRNA level can be a mass or percentage of Angptl7 mRNA per sample volume. In some embodiments, the Angptl7 mRNA level can be a mass or percentage of Angptl7 mRNA per total mRNA within the sample. In some embodiments, the Angptl7 mRNA level can be a mass or percentage of Angptl7 mRNA per total nucleic acids within the sample. In some embodiments, the Angptl7 mRNA level can be relative to another mRNA level, such as an mRNA level of a housekeeping gene, within the sample. In some embodiments, the Angptl7 mRNA measurement can be obtained by an assay such as a polymerase chain reaction (PCR) assay. In some embodiments, the PCR comprises quantitative PCR (qPCR). In some embodiments, the PCR comprises reverse transcription of the Angptl7 mRNA.

In some embodiments, the Angptl7 expression level in a sample collected from a cancer patient can be compared to a reference sample. In some embodiments, the reference sample can be from a subject without cancer. In some embodiments, the reference sample can be from the cancer patient before the cancer patient was diagnosed with cancer. In some embodiments, the reference sample can be from a second subject without cancer, wherein the second subject has previously been screened for cancer and is determined to be cancer free. In some embodiments, the Angptl7 reference level is the expression level of Angptl7 in subjects with tumors that are negative for Angptl7. In some embodiments, the reference sample can be the average Angptl7 protein level and/or the average Angptl7 mRNA level from a known population of healthy subjects, wherein the average Angptl7 protein level and/or the average Angptl7 mRNA level for the population of healthy subjects would be known or determined by one of ordinary skill in the art.

In some embodiments, functional activity of Angptl7 in a sample collected from a cancer patient can be compared to a reference sample. In some embodiments, the reference sample can be from a subject without cancer. In some embodiments, the reference sample can be from the cancer patient before the cancer patient was diagnosed with cancer. In some embodiments, the reference sample can be from a second subject without cancer, wherein the second subject has previously been screened for cancer and is determined to be cancer free. In some embodiments, the Angptl7 reference level is the functional activity of Angptl7 in subjects with tumors that are negative for Angptl7. In some embodiments, the reference sample can be the average Angptl7 protein level and/or the average Angptl7 mRNA level from a known population of healthy subjects, wherein the average Angptl7 protein level and/or the average Angptl7 mRNA level for the population of healthy subjects would be known or determined by one of ordinary skill in the art.

In some embodiments, the cancer patient can be administered a therapeutically effective dose of an Angptl7 inhibitor if the expression level of Angptl7 in the sample collected from the cancer patient is elevated compared to an Angptl7 reference level. As used herein, an “elevated” Angptl7 expression level refers to an Angptl7 protein measurement and/or an Angptl7 mRNA measurement from a cancer patient that is increased when compared to an Angptl7 reference level. In some embodiments, elevated means at least a 10%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, 1000% or greater increase compared to the Angptl7 reference level.

In some embodiments, the cancer patient can be administered a therapeutically effective dose of an Angptl7 inhibitor if the activity level of Angptl7 in the sample collected from the cancer patient is elevated compared to an Angptl7 reference level. As used herein, an “elevated” Angptl7 activity level refers to an Angptl7 activity level measurement from a cancer patient that is increased when compared an Angptl7 reference level. In some embodiments, elevated means at least a 10%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, 1000% or greater increase compared to the Angptl7 reference level.

In some embodiments, the methods described herein include measuring a level of circulating tumor cells (CTCs) in a biological sample from a cancer patient and comparing that level to a CTC reference level. In some embodiments, the CTC reference level is a CTC count above which CTCs are considered clinically elevated and at this CTC count it has been clinically proven to result in a more negative prognosis. In some embodiments, the CTC level in a biological sample from the cancer patient must exceed the CTC reference level by 10%, 20%, 30%, 40%, 50%, 60%, 70%, 80%, 90%, 100%, 125%, 150%, 175%, 200%, 225%, 250%, 275%, 300%, 325%, 350%, 375%, 400%, 425%, 450%, 475%, or more. In some embodiments, CTCs can be measured by performing a RareCyte assay or any assay that can quantify CTCs that is well-known to one skilled in the art. In some embodiments, the CTC assay quantifies the number of CTCs in a sample. One of ordinary skill in the art would be able to determine the CTC reference level.

In some embodiments, the cancer patient is determined to have an increased risk of metastatic dissemination if the CTC level in the biological sample from the cancer patient exceeds the CTC reference level. In some embodiments, the cancer patient with an increased risk of metastatic dissemination can be administered a therapeutically effective dose of an Angptl7 inhibitor.

In some embodiments, the methods described herein include performing a liquid biopsy (i.e., as understood by one skilled in the art) on the cancer patient to identify any markers that indicate tumor cell material is in the blood. In some embodiments, the liquid biopsy can measure circulating tumor DNA (ctDNA). In some embodiments, the liquid biopsy can measure exosomes. In some embodiments, the liquid biopsy can measure miRNA. In some embodiments, the markers (e.g., ctDNA, exosomes, miRNA, etc) identified through a liquid biopsy can be compared to a reference level. In some embodiments, the reference level is range for a healthy subject above which the markers (e.g., ctDNA, exosomes, miRNA, etc) identified through a liquid biopsy (i.e., from the cancer patient) are considered clinically elevated and at this range it has been clinically proven to result in a more negative prognosis. In some embodiments, the level of the marker (e.g., ctDNA, exosomes, miRNA, etc) identified by a liquid biopsy (e.g., ctDNA, exosomes, miRNA, etc) must exceed the reference level by 10%, 20%, 30%, 40%, 50%, 60%, 70%, 80%, 90%, 100%, 125%, 150%, 175%, 200%, 225%, 250%, 275%, 300%, 325%, 350%, 375%, 400%, 425%, 450%, 475%, or more.

In some embodiments, the cancer patient is determined to have an increased risk of metastatic dissemination if the markers (e.g., ctDNA, exosomes, miRNA, etc) identified through a liquid biopsy (i.e., from the cancer patient) exceed the reference level. In some embodiments, the cancer patient with an increased risk of metastatic dissemination can be administered a therapeutically effective dose of an Angptl7 inhibitor.

In some embodiments, the methods described herein include measuring a first level (i.e, baseline) of circulating tumor cells (CTCs) in a first biological sample from a cancer patient and comparing that level to a second level of CTCs from a second biological sample from said cancer patient. In some embodiments, CTCs can be measured by performing a RareCyte assay or any assay that can quantify CTCs that is well-known to one skilled in the art. In some embodiments, the CTC assay quantifies the number of CTCs in a sample. In some embodiments, the first level of CTCs in a first biological sample can be measured before the patient has cancer (i.e., before subject presents with symptoms of cancer, before subject has been diagnosed with cancer, and the like). In some embodiments, the first level of CTCs in a first biological sample can be measured shortly after the patient has presented with symptoms of cancer, shortly after the patient has been diagnosed with cancer, and the like. In some embodiments, the first level of CTCs in a first biological sample can be measured at any stage of the patient's cancer.

In some embodiments, the methods described herein can include measuring a second level of CTCs from a second biological sample from a cancer patient and comparing that level to a first CTC level from a first biological sample taken from the same subject. In some embodiments, the second biological sample is taken following the first biological sample. In some embodiments, the second biological sample can be taken days, weeks, months, or years after the first biological sample has been taken. In some embodiments, the second level of CTCs in a second biological sample can be measured before the patient has cancer (i.e., before subject presents with symptoms of cancer, before subject has been diagnosed with cancer, and the like). In some embodiments, the second level of CTCs in a second biological sample can be measured shortly after the patient has presented with symptoms of cancer, shortly after the patient has been diagnosed with cancer, and the like. In some embodiments, the second level of CTCs in a second biological sample can be measured at any stage of the patient's cancer.

In some embodiments, the methods disclosed herein can include comparing the first level of CTCs in a first biological sample to a second level of CTC in a second biological sample. In some embodiments, if the second level of CTCs in the second biological sample is not greater than the first level of CTCs from the first biological sample, a third, fourth, fifth, sixth, or, seventh, etc sample is taken until the level of CTCs in the second (or more samples) exceeds the first level of CTCs from the first biological sample. In some embodiments, the CTC level in the second (or more samples) must exceed the first level from the first biological sample by 10%, 20%, 30%, 40%, 50%, 60%, 70%, 80%, 90%, 100%, 125%, 150%, 175%, 200%, 225%, 250%, 275%, 300%, 325%, 350%, 375%, 400%, 425%, 450%, 475%, or more.

In some embodiments, the cancer patient is determined to have an increased risk of metastatic dissemination if the second (or more) level of CTCs in the second biological sample exceeds the first level of CTCs in the first biological sample. In some embodiments, the cancer patient with an increased risk of metastatic dissemination can be administered a therapeutically effective dose of an Angptl7 inhibitor.

In some embodiments, the Angptl7 inhibitor is an Angptl7 antibody. In some embodiments, the Angptl7 antibody is an Angptl7 monoclonal antibody. In some embodiments the Angptl7 antibody is an Angptl7 polyclonal antibody. The product number of the Angptl7 polyclonal antibody is PAS-36575. The antibody is publicly available.

In some embodiments, the methods disclosed herein can include administering a therapeutically effective amount of a composition comprising an Angptl7 inhibitor to a subject in need thereof. In some embodiments, the composition can be administered to inhibit necrotic core formation in a tumor to reduce the risk of metastatic dissemination from the tumor core. In some embodiments, the composition can be administered to increase survival of a cancer patient. In some embodiments, the composition can be administered to treat a necrosis-associated disease or condition. In some embodiments, the composition can be administered to inhibit tumor necrosis in a cancer patient. In some embodiments, the composition can be administered to reduce tumor cell proliferation in a cancer patient. In some embodiments, the composition can be administered to suppress tumor metastasis in a cancer patient. In some embodiments, the composition can be administered to reduce CTCs in a cancer patient.

In some embodiments, the composition comprises combining the Angptl7 inhibitor with a pharmaceutically acceptable carrier. As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In some embodiments, “pharmaceutically acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the Angptl7 inhibitor from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; lubricating agents, such as magnesium state, sodium lauryl sulfate and talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; bulking agents, such as polypeptides and amino acids serum component, such as serum albumin, HDL and LDL; and other non-toxic compatible substances employed in pharmaceutical formulations.

In some embodiments, the antibody or pharmaceutical composition can be co-administered with a second therapeutic agent. In some embodiments, the second therapeutic agent can comprise an anti-cancer therapeutic agent. In some embodiments, the anti-cancer therapeutic agent can comprise a chemotherapeutic agent, an immunotherapeutic, a small molecular inhibitor, a targeted, or an endocrine targeted therapy. In some embodiments, the chemotherapeutic agent can be doxorubicin, cyclophosphamide, paclitaxel, albumin bound paclitaxel, capecitibine, 5-fluorouracil, vinolrelbine, eribulin, irinotecan, liposomal doxorubicin (doxil), or methotrexate. In some embodiments, the small molecule inhibitors can include CDK4/6 inhibitors palbociclib, abemaciclib, ribociclib, PI3K/mTOR pathway inhibitors, alpelisib, everolimus, or HER2 targeted therapy including neratinib, and tucatinib. In some embodiments, the endocrine therapy can include tamoxifen, fulvestrant, letrozole, anastrazole, or elecasetrant. In some embodiments, the targeted biologics can include sacituzumab, trastuzumab, or antibody-drug conjugate derivatives. In some embodiments, the immunotherapy can include pembrolizumab, nivolumab, or ipilumumab.

In some embodiments, the composition is formulated as a unit dosage or other relevant form. In some embodiments, the amount of the active ingredient (i.e., Angptl7 inhibitor or second therapeutic agent) which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, from about 5 percent to about 70 percent, or from about 10 percent to about 30 percent.

In some embodiments, the administration route of the Angptl7 inhibitor or composition can be intravenous. In some embodiments, the administration route of the Angptl7 inhibitor or composition can be intraarterial, intracranial, intradermal, intraduodenal, intrammamary, intrameningeal, intraperitoneal, intratumoral, parenteral, or subcutaneous. In some embodiments, administration route of the Angptl7 inhibitor or composition is local or systemic.

The effective amount of the Angptl7 inhibitor (i.e., antibody) and or composition administered to a particular subject will depend on a variety of factors, several of which will differ from patient to patient including the activity of the specific agent(s) employed; the age, body weight, general health, sex and diet of the patient; the timing of administration, route of administration; the duration of the treatment; drugs used in combination; the judgment of the prescribing physician; and like factors known in the medical arts. Dosage amount and interval can be adjusted individually to provide plasma levels of the compound(s) which are sufficient to maintain therapeutic or prophylactic effect. In cases of local administration, the effective local concentration of the antibody cannot be related to plasma concentration. Skilled artisans will be able to optimize effective local dosages without undue experimentation.

Dosage amounts of the Angptl7 inhibitor (i.e., antibody) or pharmaceutical composition disclosed herein can typically be in the range of from about 0.0001 mg/kg/day to about 1000 mg/kg/day, but can be higher or lower, depending upon, among other factors, the activity of the antibody, its bioavailability, and various factors discussed above. In some embodiments, the dose is from about 0.0001 mg/kg to about 1000 mg/kg of body weight per day. In some embodiments, the dose is from about 0.001 mg/kg to about 1000 mg/kg of body weight per day. In some embodiments, the dose is from about 0.01 mg/kg to about 1000 mg/kg of body weight per day. In some embodiments, the dose is from about 0.1 mg/kg to about 100 mg/kg of body weight per day. In some embodiments, the dose is from about 0.5 mg/kg to about 50 mg/kg of body weight per day. In some embodiments, the dose is from about 1 mg/kg to about 25 mg/kg of body weight per day. In some embodiments, the dose is from about 5 mg/kg to about 15 mg/kg of body weight per day. In some embodiments, the dose is about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg. In some embodiments, the dose is 15 mg/kg.

The number of administrations of the Angptl7 inhibitor (i.e., antibody) or pharmaceutical composition treatments to a subject may vary. In some embodiments, introducing the antibody or pharmaceutical compositions thereof into the subject may be a one-time event. In some embodiments, such treatment may require an on-going series of repeated treatments. In some embodiments, the antibody or pharmaceutical composition is administered is administered once per day. In some embodiments, the antibody or pharmaceutical composition is administered is administered once per week. In some embodiments, the antibody or pharmaceutical composition is administered is administered once per month. In some embodiments, the antibody or pharmaceutical composition is administered is administered multiple times per day. In some embodiments, the antibody or pharmaceutical composition is administered is administered multiple times per week. In some embodiments, the antibody or pharmaceutical composition is administered is administered multiple times per month. In some embodiments, the antibody or pharmaceutical composition treatment are administered over the course of set dosing schedule. The exact protocols depend upon the disease or condition, the stage of the disease and parameters of the individual subject being treated as determined by one of ordinary skill in the art.

The treatment duration of the Angptl7 inhibitor (i.e., antibody) or pharmaceutical composition to a subject can vary. In some embodiments, the antibody or pharmaceutical composition is administered (e.g., intravenously) for at least one hour, at least two hours, at least three hours, at least four hours, at least five hours, at least six hours, at least seven hours, at least eight hours, at least nine hours, or at least ten hours. The exact protocols depend upon the disease or condition, the stage of the disease and parameters of the individual subject being treated as determined by one of ordinary skill in the art.

The course of treatment of the Angptl7 inhibitor (i.e., antibody) or pharmaceutical composition to a subject can vary. In some embodiments, the treatment duration is 1 week, 1 month, or 1 year. In other embodiments the treatment duration can be multiple weeks, multiple months, or multiple years. The exact protocols depend upon the disease or condition, the stage of the disease and parameters of the individual subject being treated as determined by one of ordinary skill in the art.

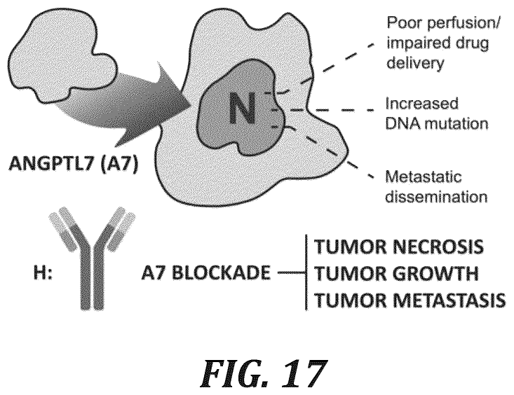

In some embodiments, treating refers to the treatment of a disease in a mammal, e.g., in a human, including (a) inhibiting the disease, i.e., arresting disease development or preventing disease progression; (b) relieving the disease, i.e., causing regression of the disease state or relieving one or more symptoms of the disease; and (c) curing the disease, i.e., remission of one or more disease symptoms. In some embodiments, treatment results in an improvement or remediation of the symptoms of the disease. In some embodiments, treatment may refer to a short-term (e.g., temporary and/or acute) and/or a long-term (e.g., sustained) improvement or remediation in one or more disease symptoms. In some embodiments, the improvement is an observable or measurable improvement. In some embodiments, the improvement is an improvement in the general feeling of well-being of the subject. In some embodiments, administration of the Angptl7 inhibitor (i.e., antibody) or pharmaceutical composition disclosed herein can reduce tumor cell proliferation in a cancer patient. In some embodiments, administration of the Angptl7 inhibitor (i.e., antibody) or pharmaceutical composition disclosed herein can inhibit tumor necrosis. In some embodiments, administration of the Angptl7 inhibitor (i.e., antibody) or pharmaceutical composition disclosed herein can suppress tumor metastasis. In some embodiments, administration of the Angptl7 inhibitor (i.e., antibody) or pharmaceutical composition disclosed herein can reduce CTCs. In some embodiments, administration of the Angptl7 inhibitor (i.e., antibody) or pharmaceutical composition disclosed herein can inhibit necrotic core formation in a tumor to reduce the risk of metastatic dissemination from the tumor core. In some embodiments, administration of the Angptl7 inhibitor (i.e., antibody) or pharmaceutical composition disclosed herein can increase survival of a cancer patient. In some embodiments, administration of the Angptl7 inhibitor (i.e., antibody) or pharmaceutical composition disclosed herein can treat a necrosis-associate disease or condition. In some embodiments, administration of the Angptl7 inhibitor (i.e., antibody) or pharmaceutical composition disclosed herein can reduce at least one indication of aggressive tumor cell behavior. FIG. 17 (illustrating that aggressive tumors undergo necrosis promoting drug resistance, tumor evolution, and metastasis). In some embodiments, administration of the Angptl7 inhibitor (i.e., antibody) or pharmaceutical composition disclosed herein can reduce expression of Angtptl7 protein in a tumor. In some embodiments, administration of the Angptl7 inhibitor (i.e., antibody) or pharmaceutical composition disclosed herein can reduce expression of Angtptl7 mRNA in a tumor. In some embodiments, administration of the Angptl7 inhibitor (i.e., antibody) or pharmaceutical composition disclosed herein can reduce the in vivo active level of A7. In some embodiments, administration of the Angptl7 inhibitor (i.e., antibody) or pharmaceutical composition disclosed herein can reduce vascular leakiness. In some embodiments, administration of the Angptl7 inhibitor (i.e., antibody) or pharmaceutical composition disclosed herein can increase drug (i.e., a second therapeutic) penetration into a tumor.

In some embodiments, at least one indication of aggressive tumor cell behavior comprises necrosis formation in the tumor core; metastasis formation; or an increase in CTCs. In some embodiments, administration of the Angptl7 inhibitor (i.e., antibody) or pharmaceutical composition disclosed herein can reduce at least one indication of tumor cell proliferation. In some embodiments, at least one indication of tumor cell proliferation comprises necrosis formation in the tumor core; metastasis formation; or an increase in CTCs.

### EXAMPLES

### Example 1

This Example describes the use of a rat transplantation model to uncover a temporal correlation between tumor dissemination and the formation of large contiguous zones of necrosis within the tumor core. These necrotic zones harbored dilated blood vessels and intravascular tumor cells. Importantly, as described further below, a tumor-cell secreted factor angiopoietin like-7 (Angptl7) regulates formation of necrosis. The inventors' investigation has identified a targetable axis of the necrosis ecosystem to suppress metastatic dissemination from the tumor interior.

A Low-to-High Circulating Tumor Cell (CTC) Transition Occurs in a Rat Transplantation Model of Breast Cancer

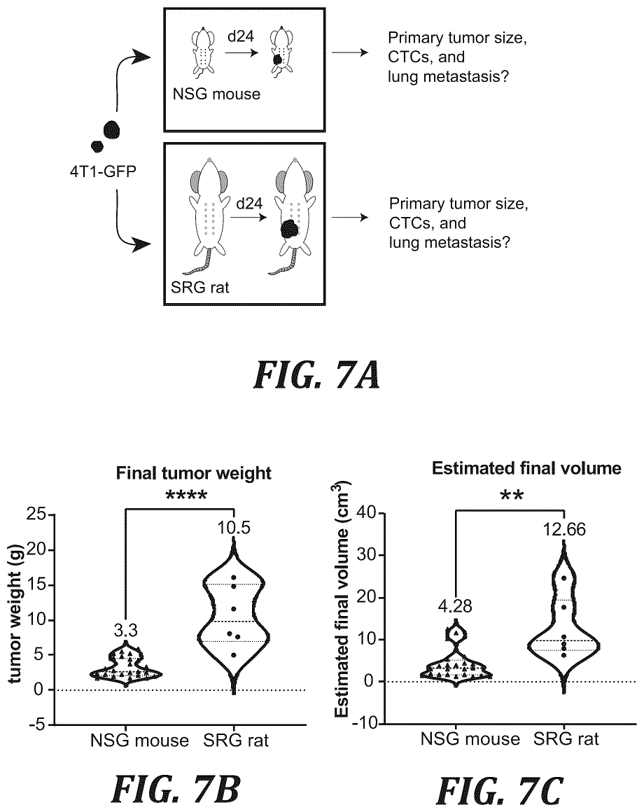

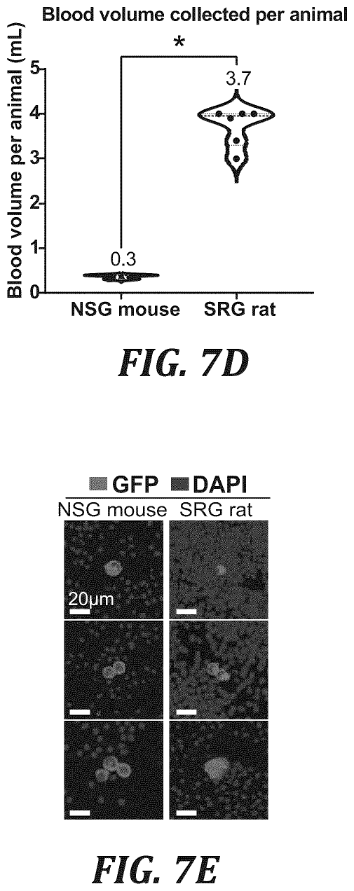

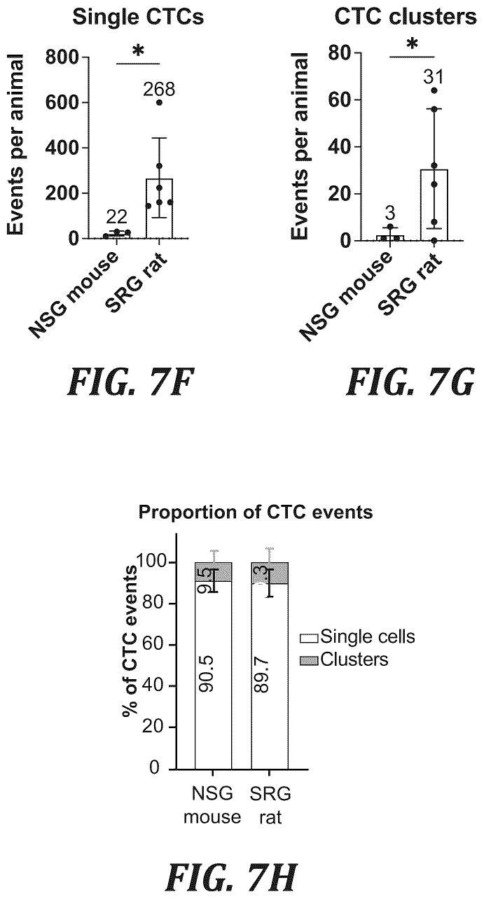

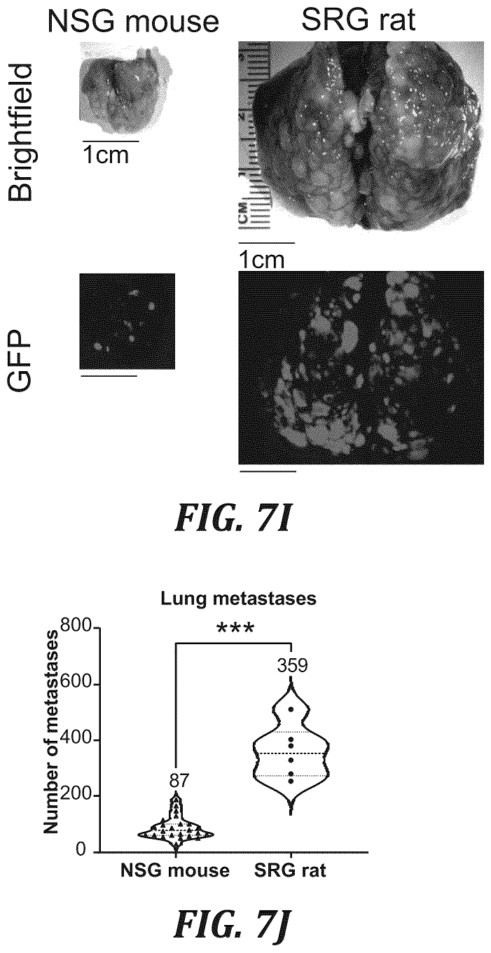

Isolating tumor cells in transit is painstaking in foundational models for cancer metastasis research including mouse, zebrafish, and chick embryo. To avoid this issue, the use of rats, a larger animal model, would enable more robust detection of dissemination events locally and systemically in the circulation. As such, SCID rats on the Sprague-Dawley background were employed with a double knockout for the Rag2 and Il2rgamma genes [denoted SRG]. SRG rats are deficient in T cells, B cells, and NK cells similar to NOD scid gamma (NSG) mice. To compare the performance of these two transplantation models, 4T1 tumor cells, a mouse mammary tumor model of triple-negative breast cancer, transduced with a GFP reporter, were transplanted into the mammary fat pad of SRG rats or NSG mice and collected all tumors synchronously once maximum tumor size was reached for mice, which was 2 cm (FIG. 7A). Altogether, SRG rats produced three times larger tumors by weight and by estimated tumor volume (FIGS. 7B and 7C). Ten times more blood was collected with SRG rats, which in turn yielded 10 times more single CTCs and CTC-clusters per animal (FIGS. 7D-G). For both CTCs and CTC-clusters, morphologic appearance and relative proportions in blood were identical between SRG rats and NSG mice (FIGS. 7E and 7H). Consistent with the increased CTC abundance, lung metastases counted by stereomicroscopy were four times more abundant in SRG rats (FIGS. 7I and 7J). Because the same number of 4T1 cells was transplanted in both SRG rats and NSG mice and because tumors were harvested at the same time, direct comparison showed that the rat transplantation model increases the efficiency of detecting rare tumor dissemination events by 10-fold.

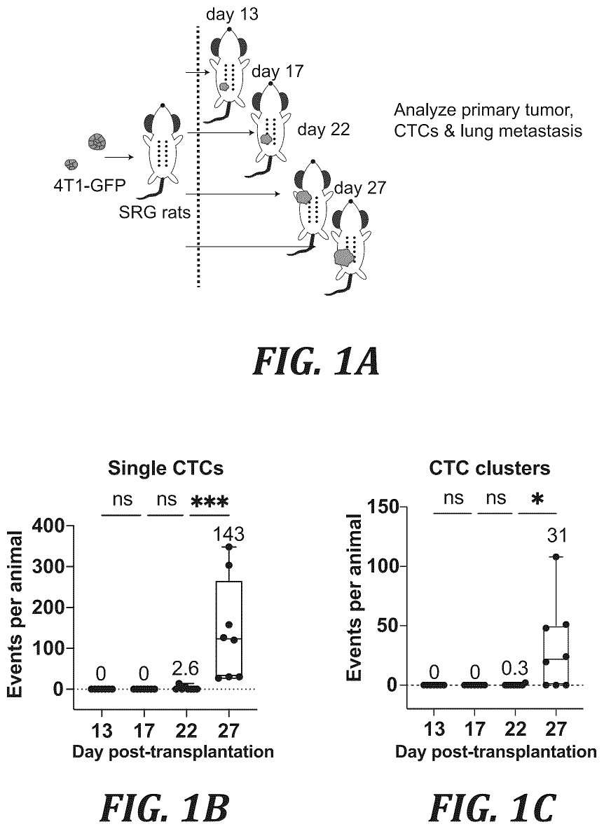

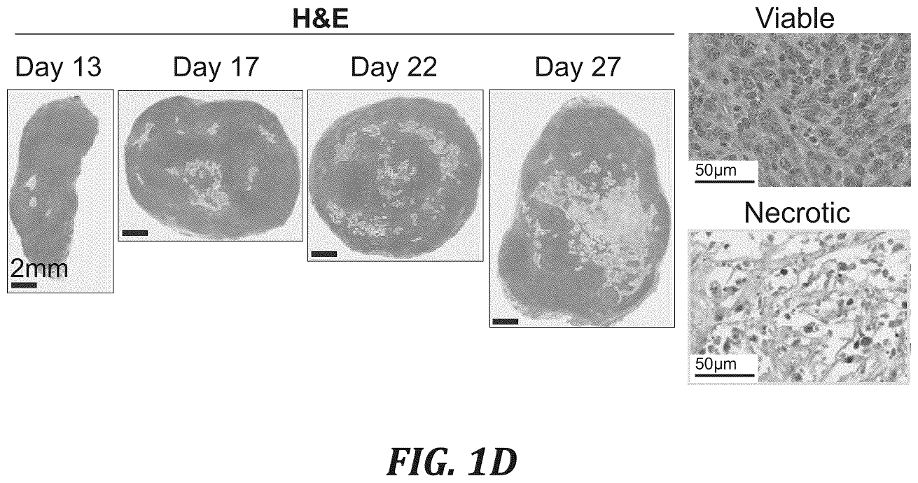

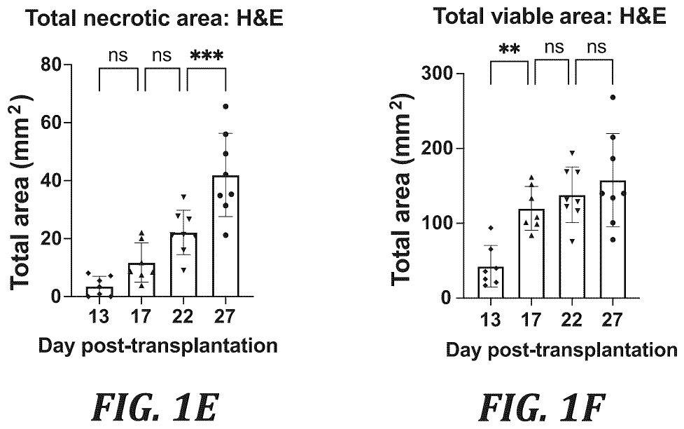

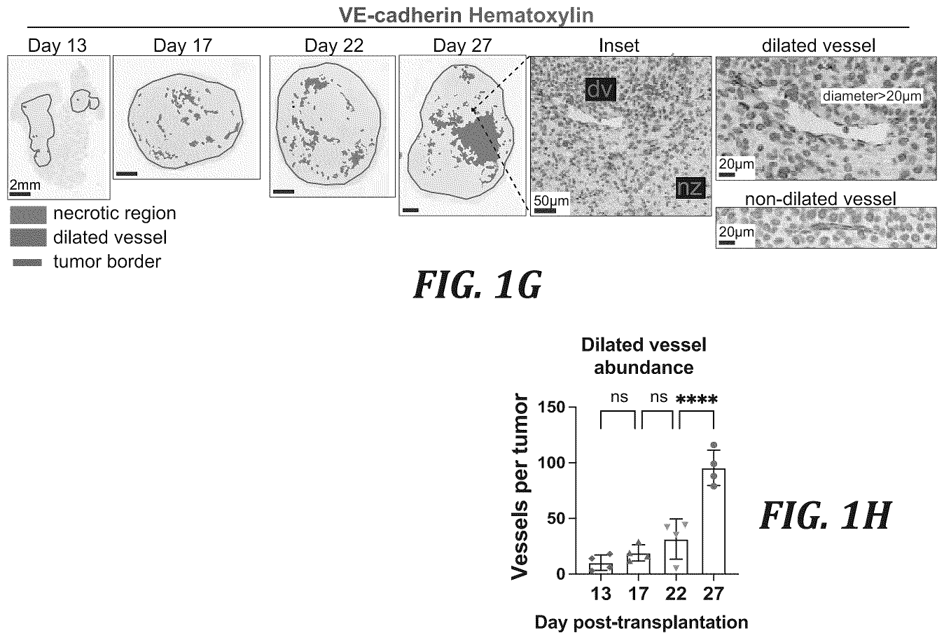

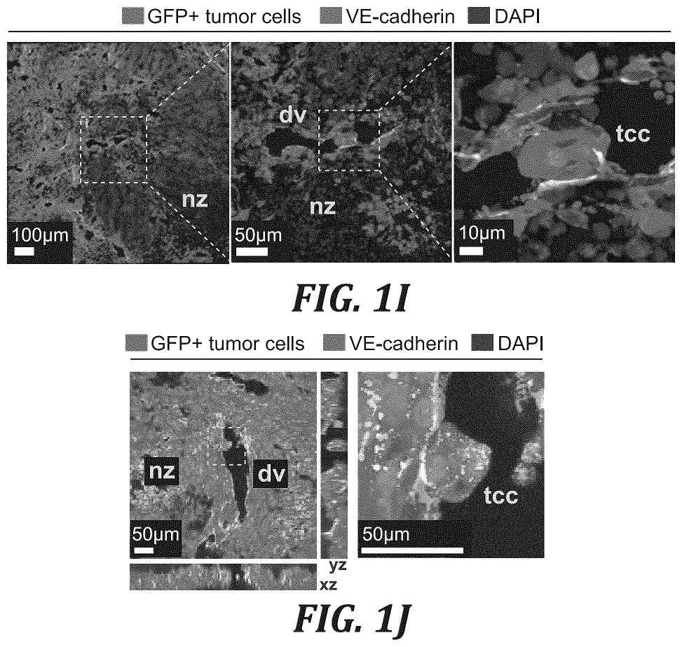

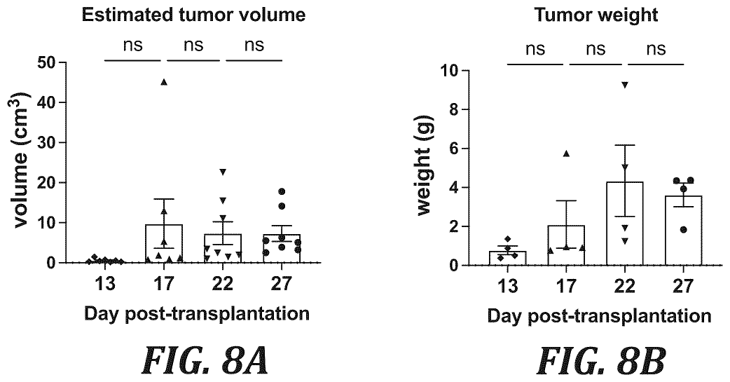

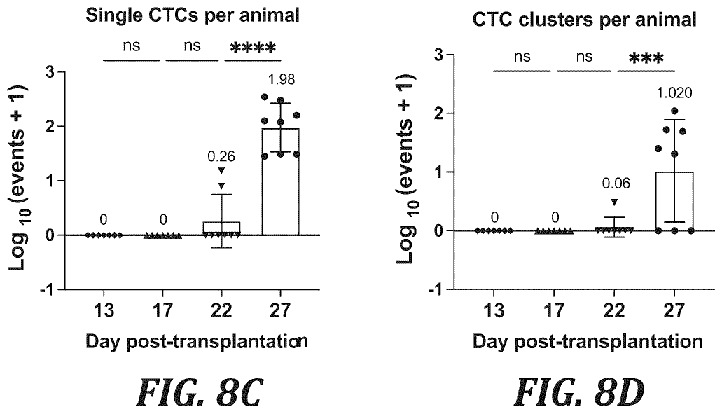

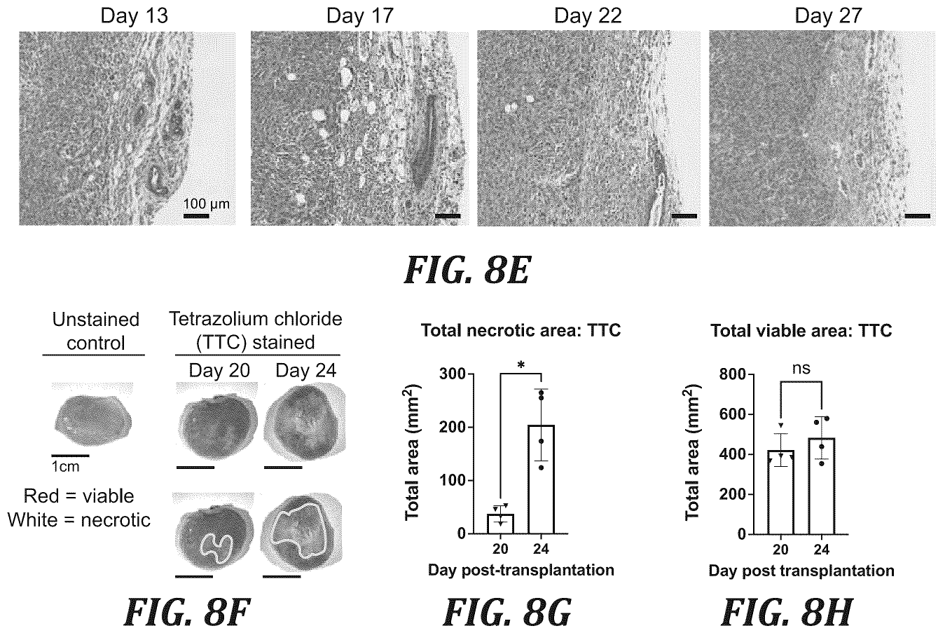

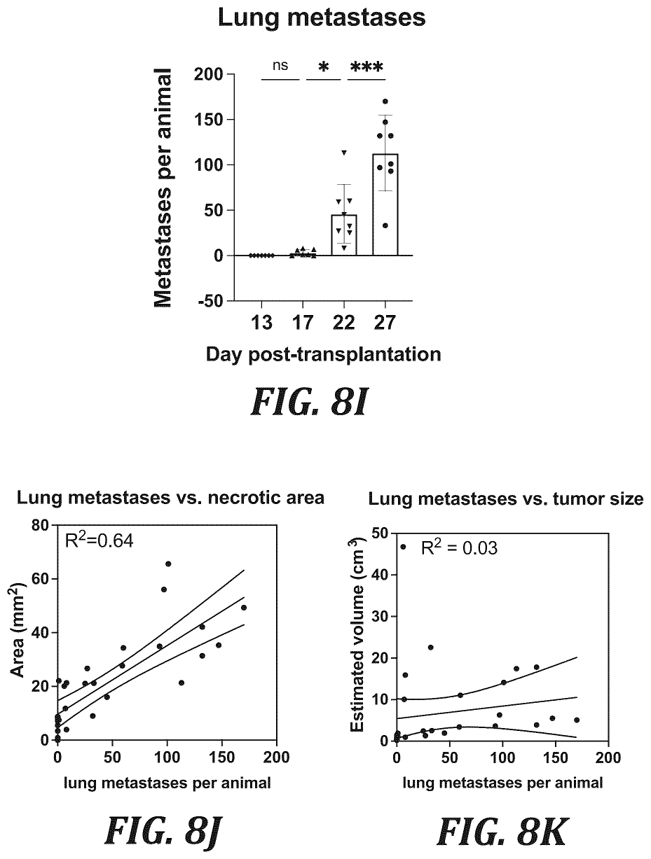

Having identified the rat xenograft model as an efficient system to study CTC dissemination, this model was used to determine CTC abundance temporally. GFP-labeled 4T1 cells were transplanted into the mammary fat pad of SRG rats (FIG. 1A). Blood, lungs, and tumors were harvested at days 13, 17, 22, and 27 post-transplantation. In this model, tumor size increased significantly between days 13 and 17 with evidence of growth plateau and significant variance in tumor size between SRG rats by both estimated tumor volume and by tumor weight (FIGS. 8A and 8B). Surprisingly in the same time frame, a pronounced nonlinear increase in CTC abundance was observed. Specifically, between days 22 and 27, a ˜50-fold increase in CTCs and ˜10-fold increase in CTC clusters was identified (FIGS. 1B, 1C, and FIGS. 8C, 8D).

The Low-to-High CTC Transition Is Associated with a Necrotic Tumor Core, Dilated Vessels, and Perinecrotic Tumor Cell Vascular Invasion

Given the robust increase in CTC abundance that was uncorrelated with primary tumor burden, we next asked how primary tumors were changing on a cellular level. Hematoxylin and eosin (H&E) staining of tumors revealed tumors that were histologically indistinguishable at the tumor-invasive border over time (FIG. 8E). Instead, H&E revealed robust changes in the tumor core with appearance of confluent zones of central necrosis. Total necrotic area in the primary tumor increased over time with marked elevation between days 22 and 27, whereas total viable area increased early and subsequently plateaued (FIGS. 1D-F). As a second method, tetrazolium chloride assay, a colorimetric method for determining cell viability, confirmed that total necrotic area, but not total viable area, increased markedly at the late time point FIGS. 8F-H). Likewise, the number of lung metastases per animal was highly correlated with necrotic area, but not tumor volume (FIGS. 8I-K).

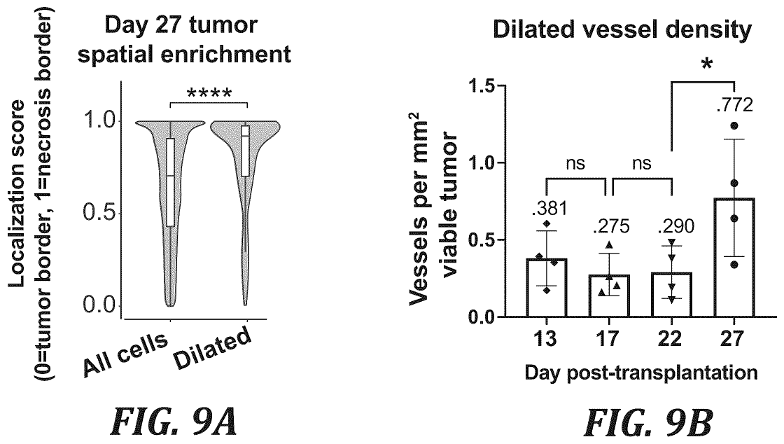

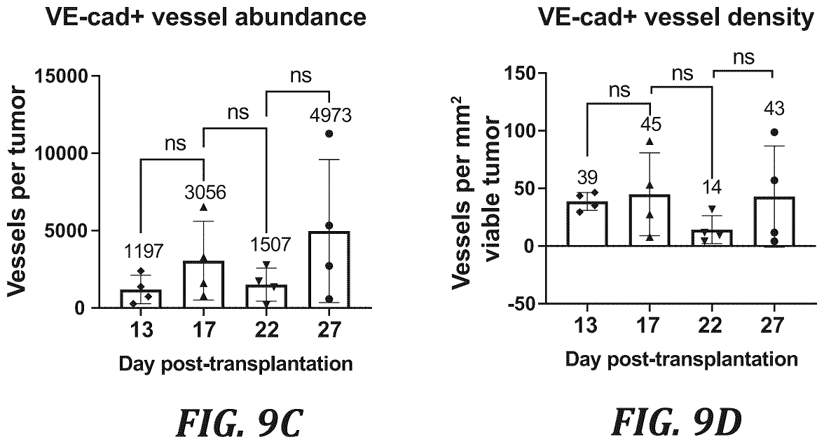

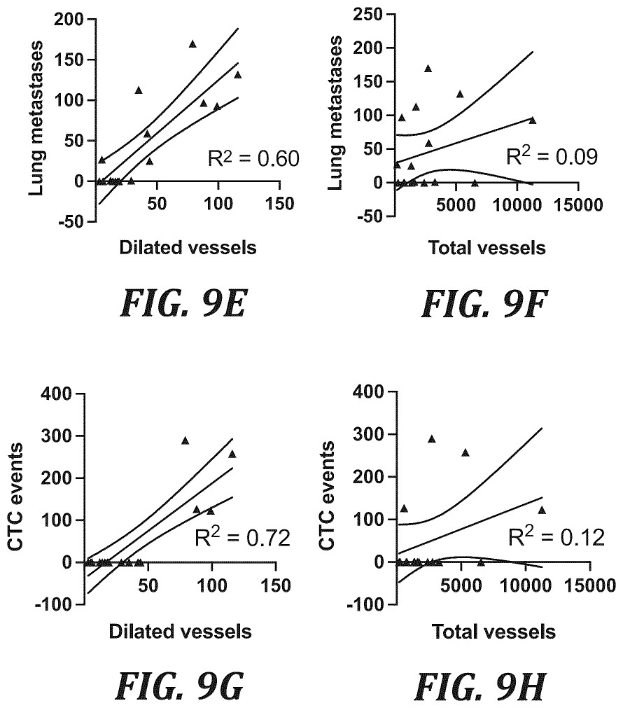

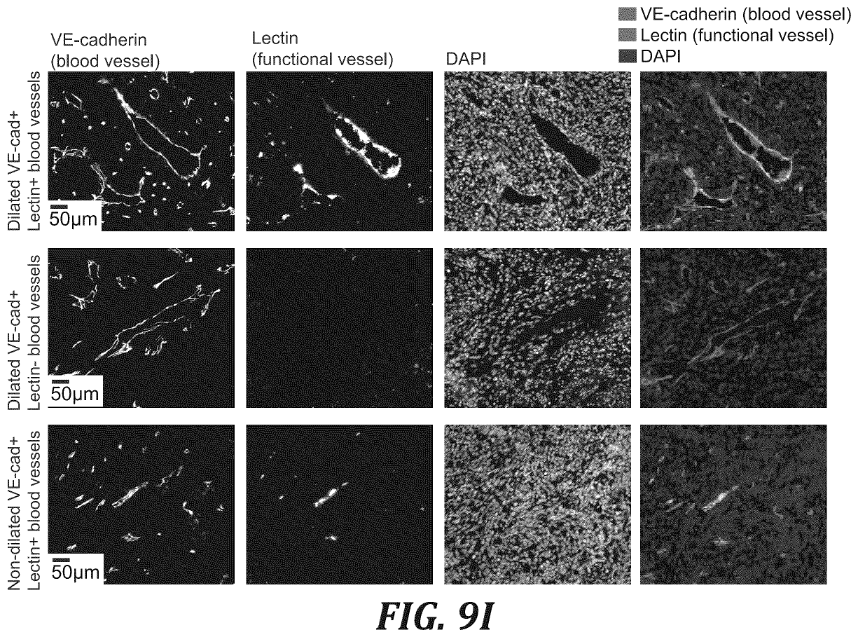

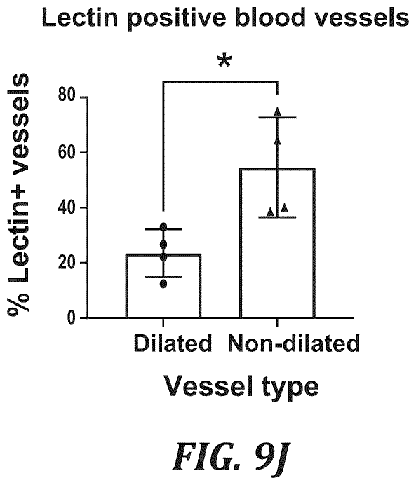

Extending these results further, tumors were stained for VE-cadherin to mark blood vessels. VE-cadherin staining demonstrated abnormal dilated blood vessels, penetrating the necrotic core, that was markedly increased in abundance and density between days 22 and 27 (FIGS. 1G, 1H and FIGS. 9A, 9B). In contrast, neither total microvascular abundance nor density increased over the same time points (FIGS. 9C and 9D). The number of lung metastases per animal and CTCs per animal were highly correlated with the number of dilated vessels but not total vessels (FIGS. 9E-H). Functionally, dilated vessels were less often labeled by fluorescent lectin compared with nondilated vessels, indicative of differences in blood flow between vessel types (FIGS. 9I and 9J). Further, dilated vessels contained GFP+ multicellular tumor cell clusters, consistent with vascular invasion (FIGS. 1I and 1J). Together, these data reveal an acute low-to-high CTC transition that coincides with robust changes in the tumor core, not rim. These tumor core alterations included confluent zones of necrosis and necrosis-adjacent dilated blood vessels containing intravascular tumor cells.

Transcriptome Profiling Comparing Tumor Core and Rim Reveal That Angptl7 Is a Tumor-Specific, Core-Enriched Factor Localized to the Perinecrotic Zone

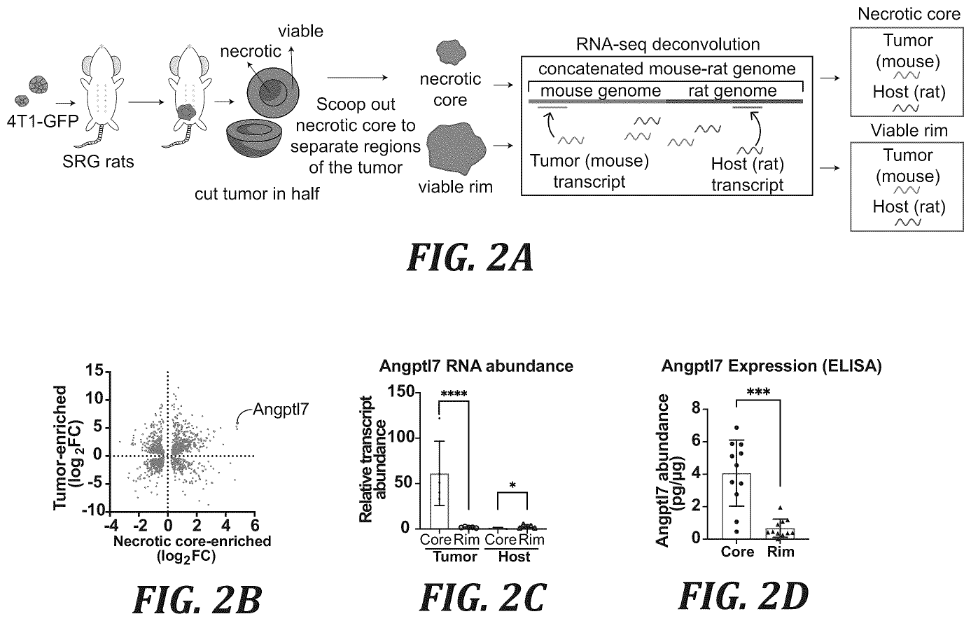

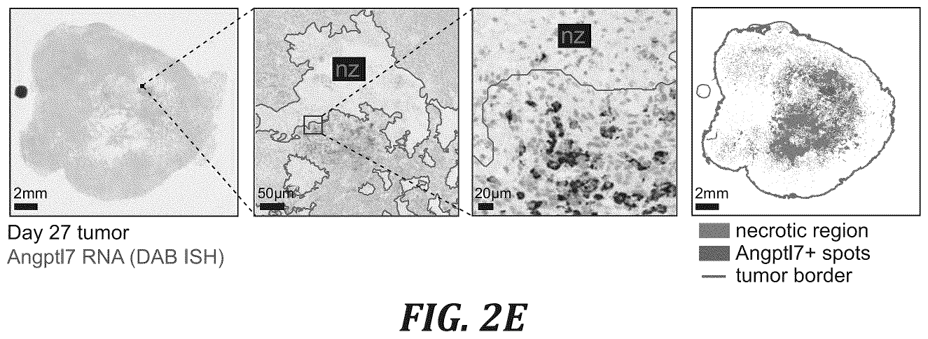

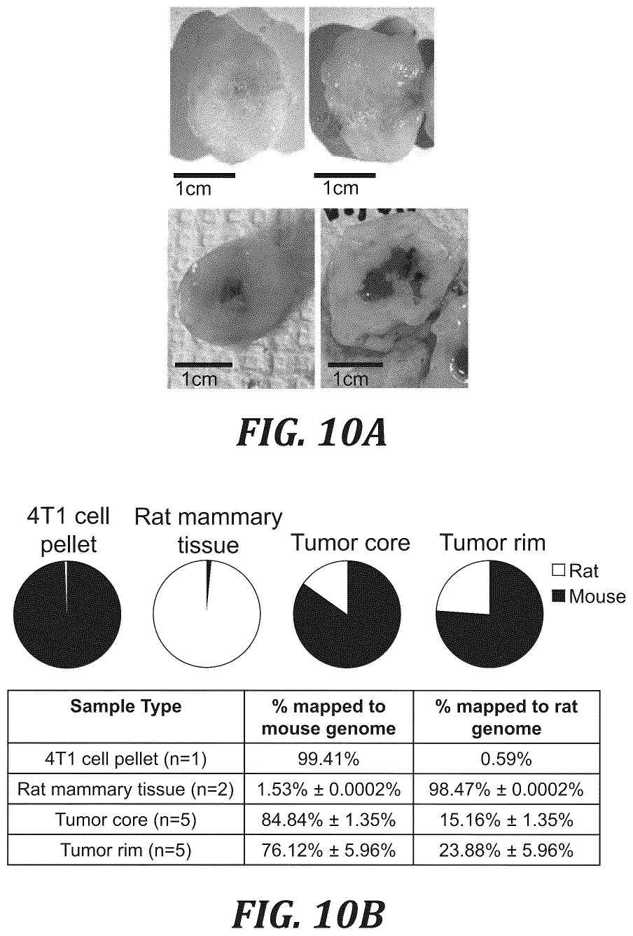

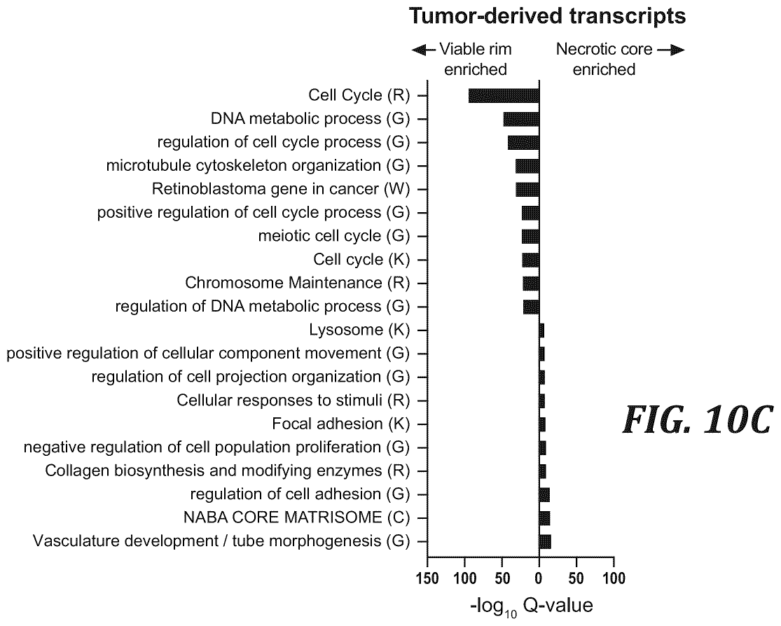

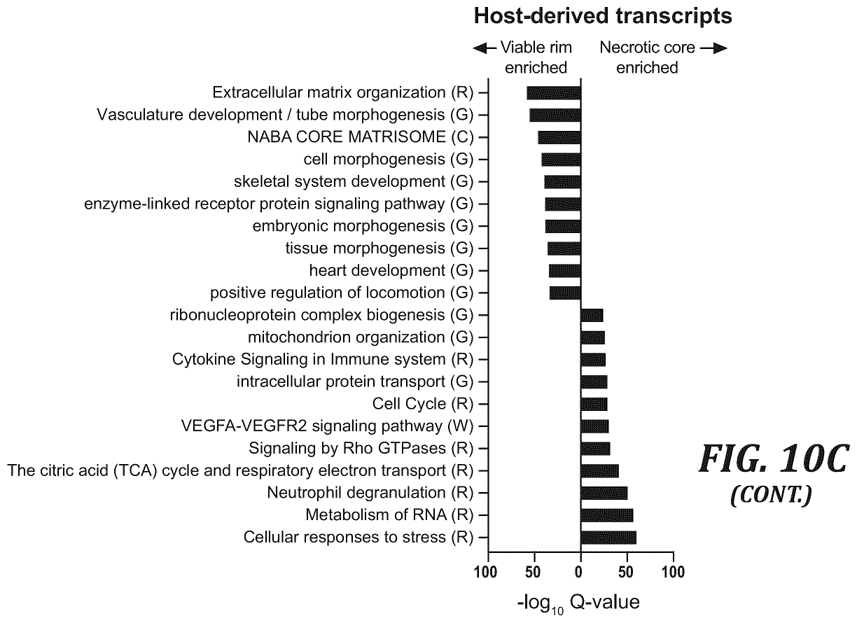

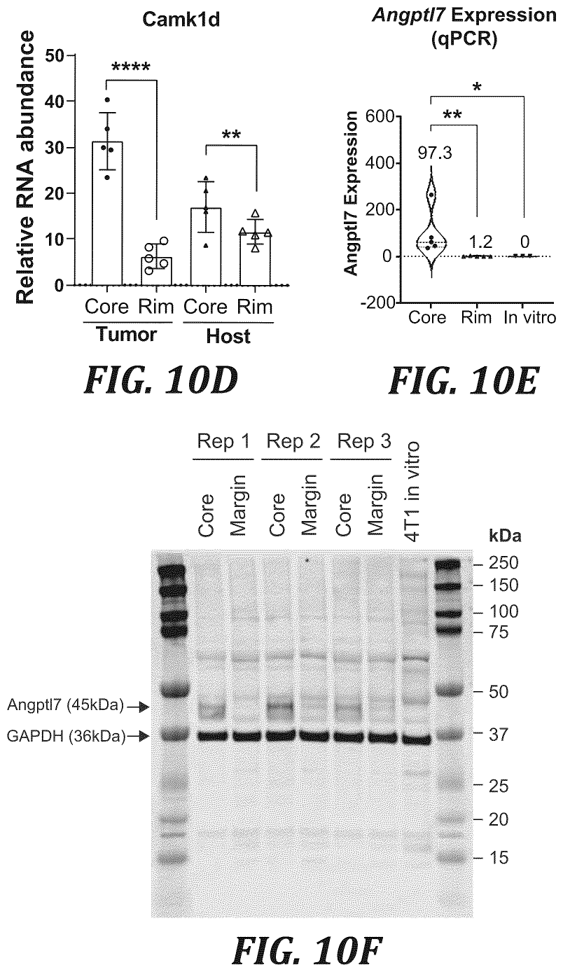

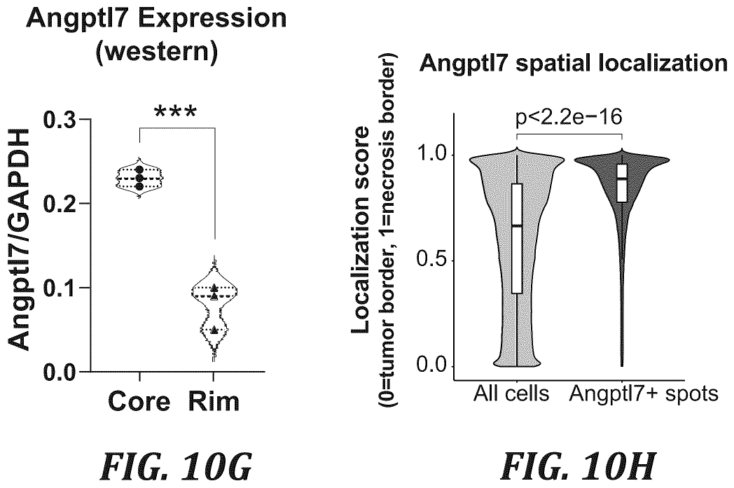

The inventors sought to define the molecular changes defining the necrotic core. To this end, the tumor core was dissected from the rim, and interrogated gene expression originating from tumor or from host cells (FIG. 2A). In this regard, the properties of the rat transplant model and the use of a mouse-in-rat xenograft were advantageous. At time of harvest, rat tumors were 3 cm in diameter or larger and had distinct tissue morphology between the firmer tumor rim and softer, pinker tumor core, that was in some cases also obviously liquified (FIG. 10A), enabling macrodissecting the necrotic core from the nonnecrotic rim. Following RNA-sequencing of macrodissected regions, sequence reads were further aligned to a concatenated rat-mouse combined genome, and deconvoluted to obtain tumor-derived gene expression (originating from mouse) vs. host-derived gene expression (originating from the rat). This deconvolution algorithm robustly separated mouse and rat RNA transcriptomes and revealed that 76% of reads were mouse-in-origin in tumor rim, and 84% of reads were mouse-in-origin in tumor core (FIG. 10B), indicating the majority of reads were tumor-derived.

Gene-ontology analysis further revealed distinct patterns of ontology enrichment between tumor and host, and between rim and core (FIG. 10C). In the host transcriptome, gene sets typically associated with the invasive border were up in the rim (including extracellular matrix, vascular morphogenesis, and locomotion), while gene sets associated with tissue necrosis were up in the core (including cellular stress response, RNA metabolism, and neutrophil degranulation). Likewise, in the tumor transcriptome, gene sets associated with replicating cells were up in the rim (cell cycle, DNA replication), while gene sets involved in vascular remodeling appeared up in the core (vasculature development/morphogenesis, matrisome, and regulation of cell adhesion).

These gene expression patterns revealed that tumor cells in the interior actively express genes associated with vascular tube morphogenesis. However, these core genes could be expressed in both tumor and host compartments. To illustrate, the top differentially expressed tumor-derived gene was Camkld. However, host-derived Camkld was also expressed in both core and rim; therefore, Camkld is not tumor-specific (FIG. 10D). To identify top core-enriched tumor-specific genes, interspecies analysis was performed to find genes that were specifically mouse tumor-derived and not expressed in the rat host (FIG. 2B). The top enriched factor from this analysis was angiopoietin-like 7 (Angptl7), enriched 32-fold in tumor vs. host, and over 16-fold in tumor core vs. tumor rim (FIG. 2C). The enrichment for Angptl7 in the tumor core was further confirmed by qPCR, western blot, and ELISA specific for mouse Angptl7 (FIG. 2D and FIG. 10E—G). In addition, RNA in situ hybridization (ISH) was performed for mouse Angptl7 (FIG. 2E and FIG. 10H). To quantify spatial enrichment, the distance of every spot was calculated relative to the nearest tumor-stromal border or tumor-necrotic interface. Angptl7 was markedly enriched in a perinecrotic distribution in the tumor core and with little expression in the tumor rim. These findings show that Angptl7 is a tumor-derived factor enriched in the core of high-CTC tumors, and spatially localized to the perinecrotic zone.

Angptl7 Suppression Markedly Normalizes Histologic Necrosis in the Tumor Core and Reduces the Number of CTCs and Metastases

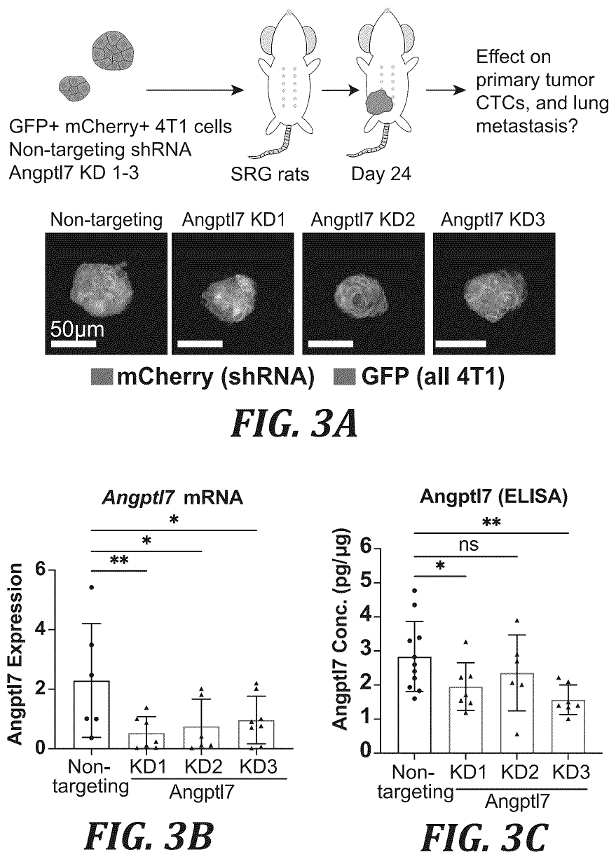

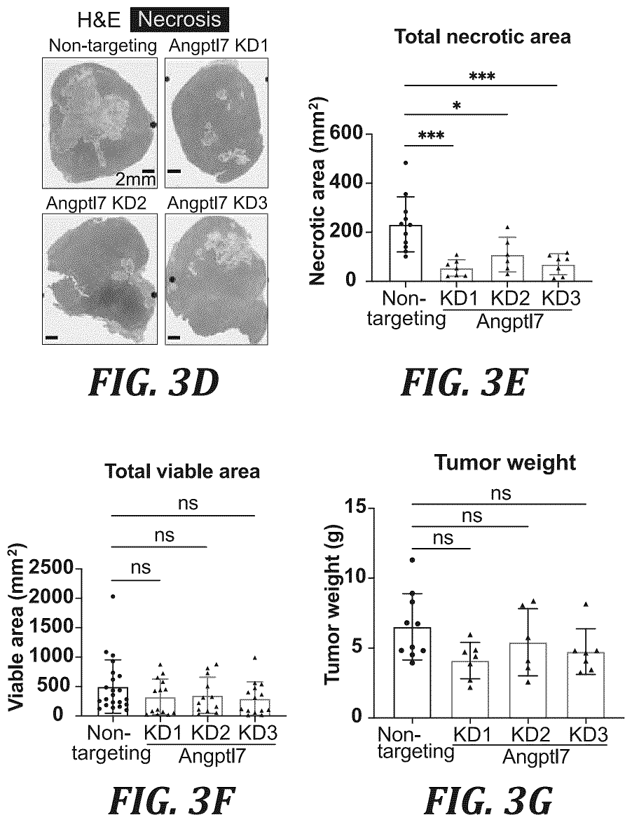

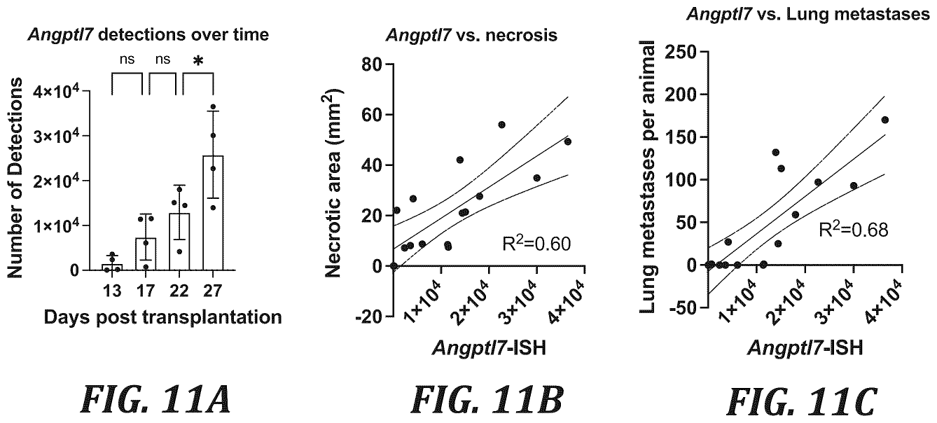

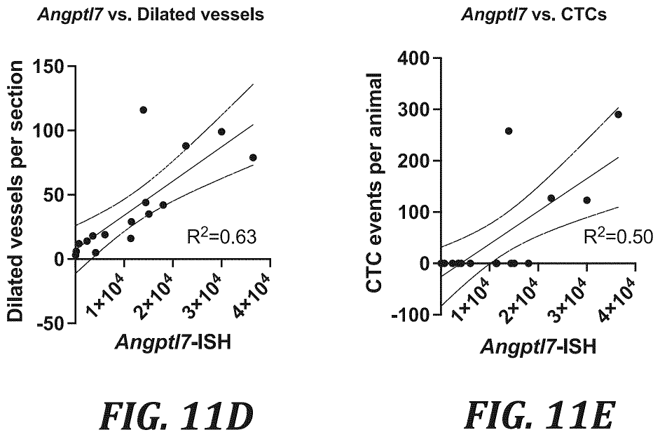

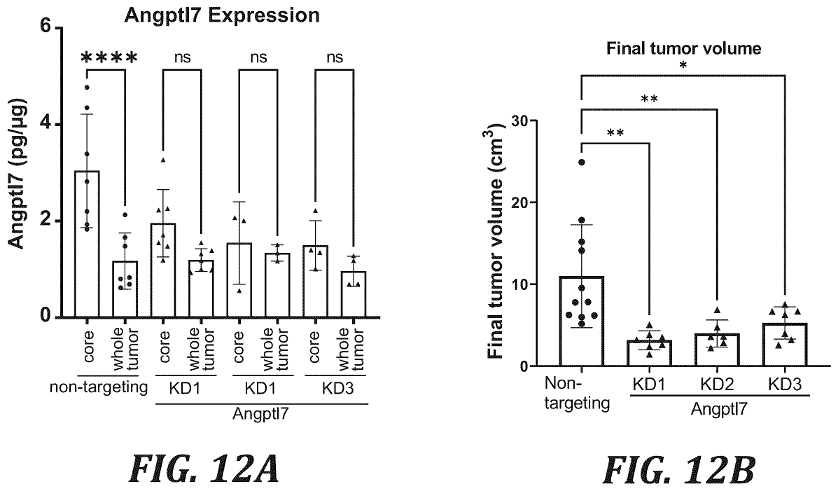

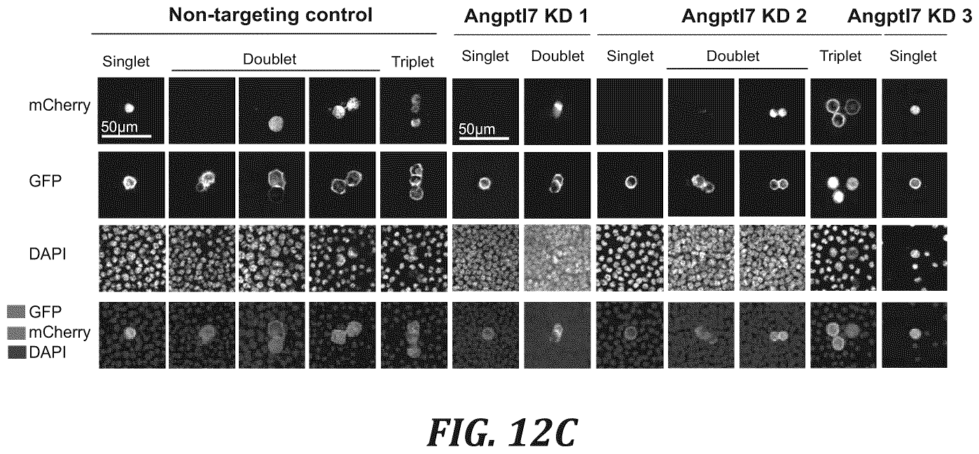

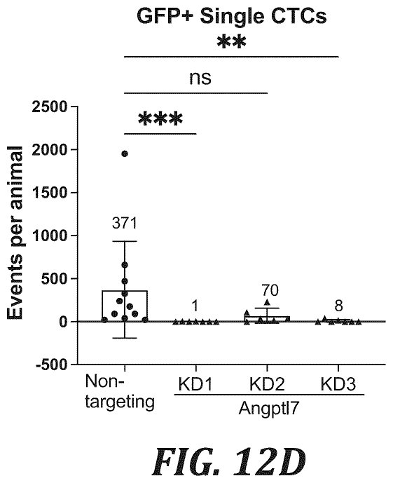

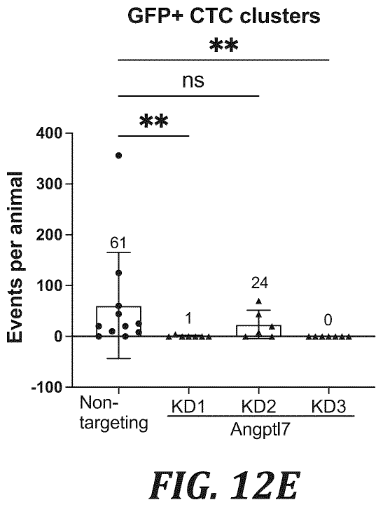

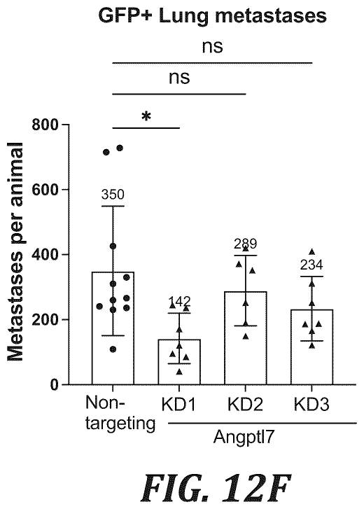

ANGPTL7 belongs to a family of secreted proteins structurally related to the angiopoietins and that have been implicated in lipid metabolism, cardiovascular disease, stem cell renewal, and cancer. Compared with other ANGPTL proteins, ANGPTL7 is among the least characterized, being predominantly expressed in the human cornea, and elevated in the aqueous humor of the eye in patients with glaucoma, a disease of increased intraocular pressure. In the rat model, the number of Angptl7 RNA detections was observed to increase markedly over time and was highly correlated with total necrotic area, dilated vessels, CTCs, and lung metastases (FIGS. 11A-E). Given that strong spatial and temporal association was observed between Angptl7 expression and the necrotic core, it was next determined whether Angptl7 is required for necrotic core formation and metastatic dissemination. To answer this question, 4T1 tumor cells were transduced with a puromycin selectable membrane-GFP lentiviral vector and a second blasticidin selectable lentivirus encoding for shRNA hairpin and expressing cytoplasmic mCherry to generate stably transduced nontargeting control and Angptl7 knockdown (KD) lines expressing both fluorescent reporters (FIG. 3A). No differences in survival, proliferation, or ability to form clusters were observed between conditions during in vitro culture. Nontargeting control knockdown lines and Angptl7-knockdown (KD1-3) were each transplanted orthotopically into SRG rats. Because Angptl7 expression was undetectable in vitro (FIG. 10E), but highly induced in vivo, knockdown efficiency was confirmed in harvested tumor. By qPCR, Angptl7 RNA expression was markedly reduced with all three knockdown KDs (FIG. 3B). Because Angptl7 expression is low in whole tumor lysates, knockdown was confirmed on a protein level by ELISA of tumor interior, and Angptl7 protein expression was significantly reduced in KD1 and KD3 in the tumor interior, where Angptl7 expression is higher than in whole tumor lysates (FIG. 3C and FIG. 12A).

Having confirmed knockdown, tumor histology was evaluated by H&E staining. Strikingly, knockdown of Angptl7 induced between 53 and 77% reduction in the area of necrotic cores (FIGS. 3D and 3E). One possibility is that decreasing necrosis could promote tumor growth, a tradeoff observed in prior studies with perturbations improving vascularization (Donato C, Kunz L, Castro-Giner F, et al. Hypoxia Triggers the Intravasation of Clustered Circulating Tumor Cells. Cell Reports. 2020; 32(10):108105; Carmeliet P, Jain R K. Angiogenesis in cancer and other diseases. Nature 2000 407:6801. 2000; 407(6801):249-257). However, suppression of Angptl7 did not increase either total viable area, tumor weight, or tumor volume, indicating that Angptl7 suppression does not promote tumor growth (FIGS. 3F and 3G and FIG. 12B).

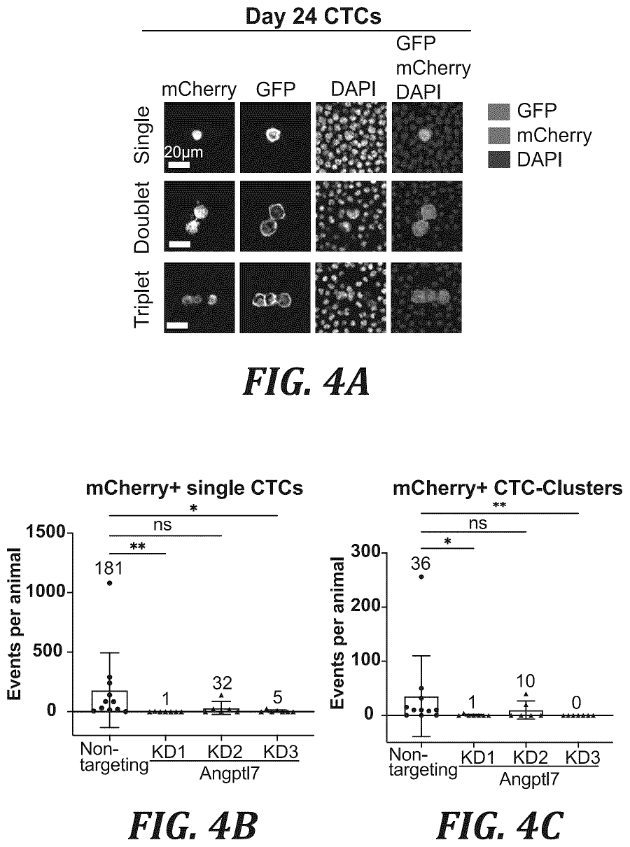

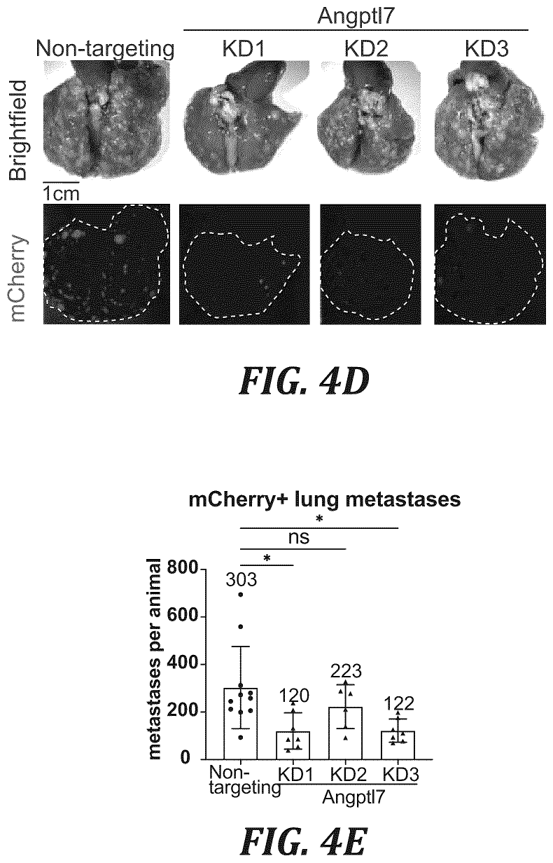

Given these effects on the histology of the tumor core, the effect of Angptl7 knockdown was evaluated on CTC abundance and metastasis. Remarkably, drastic reductions were observed in both mCherry+ single-CTC and CTC-cluster abundance, which reduced from an average of 181 single CTCs down to 1 single CTC, and from 36 CTC-clusters down to 0 or 1 CTC-clusters for the best knockdowns (FIGS. 4A-C and FIG. 12C). Likewise, the number of mCherry+ lung metastases was also markedly reduced in Angptl7 knockdown conditions (FIGS. 4D and 4E). For the different knockdowns, the degree of necrosis suppression correlated with degree of CTC dissemination and metastasis suppression. Likewise, reductions were observed in GFP+ CTC, CTC cluster, and lung metastasis abundance (FIGS. 12D-F). Taken together, these data establish that Angptl7, a tumor-specific factor that is specifically expressed in the tumor interior, is necessary for necrosis formation in the tumor core, dissemination of both single and clustered CTCs, and lung metastasis.

Angptl7 Suppression Normalizes a Subset of Tumor Core Gene Expression and Regulates Blood Vessel Morphology and Vascular Permeability

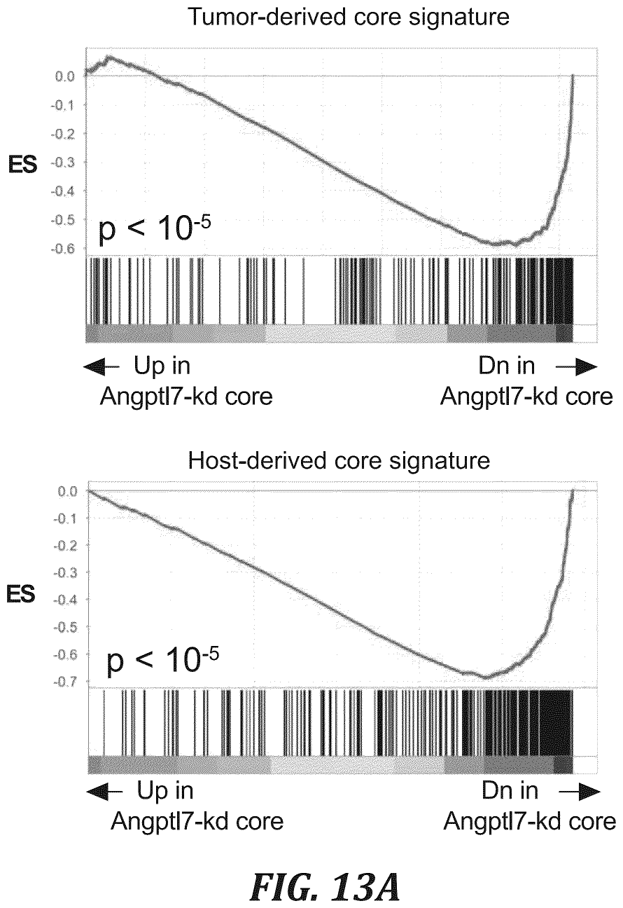

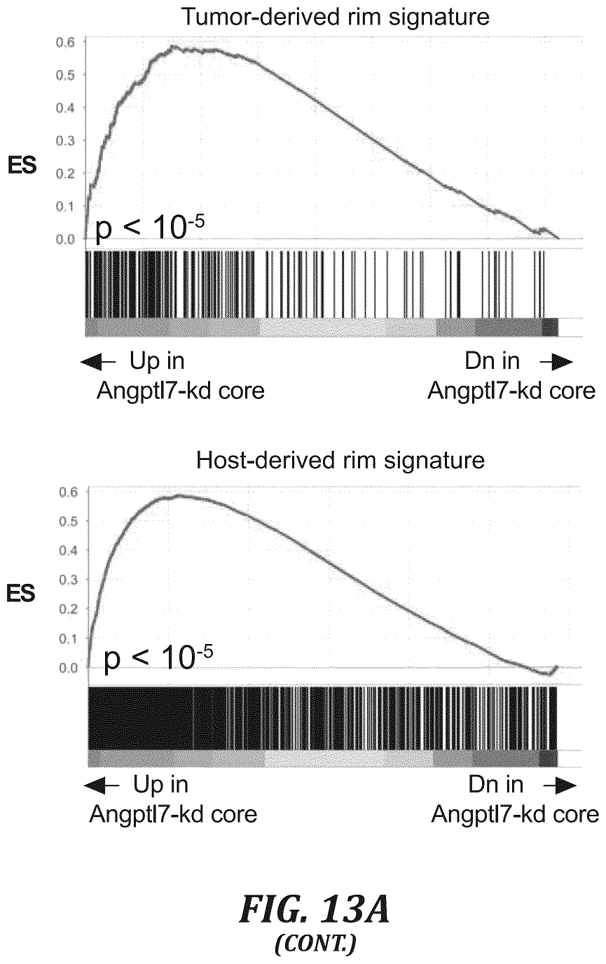

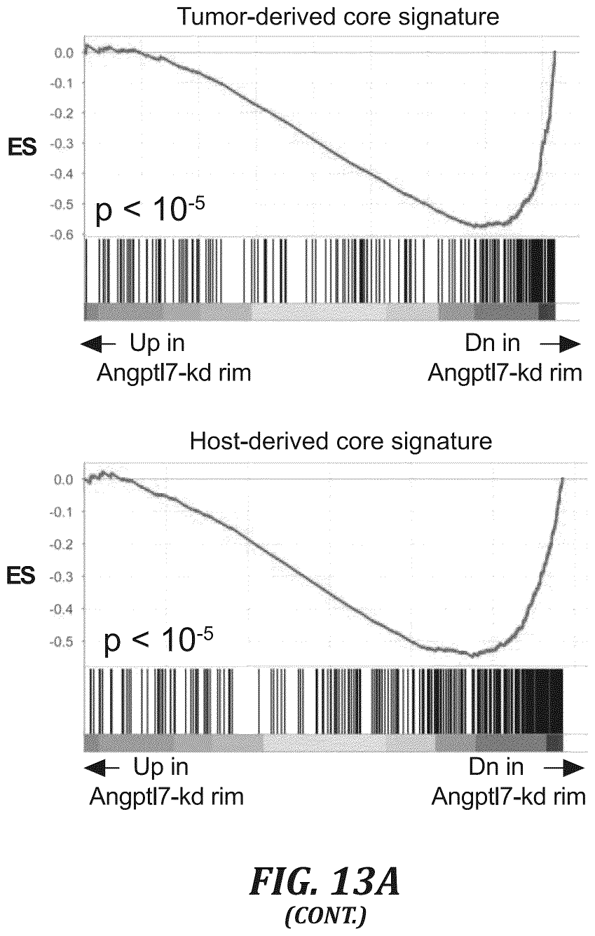

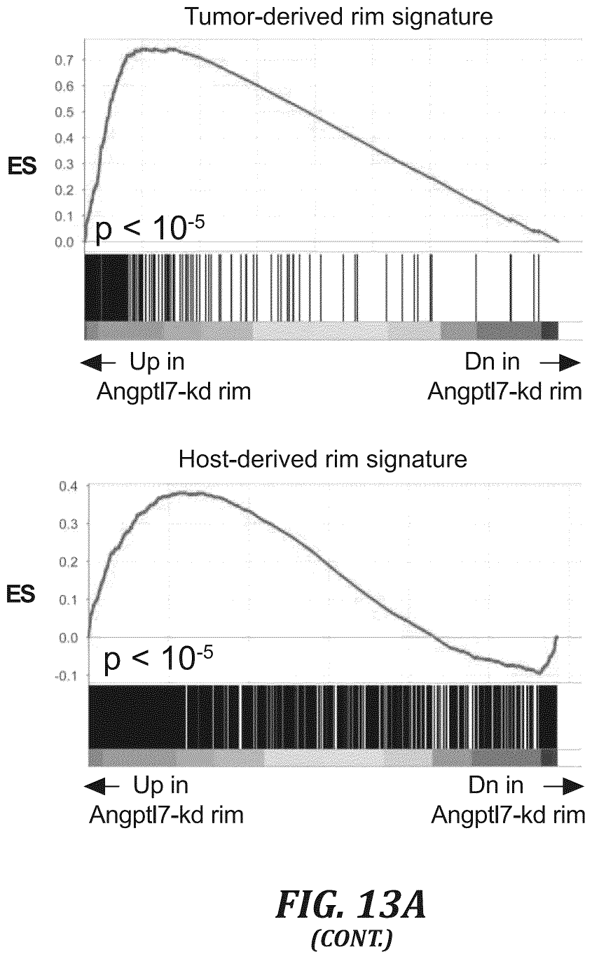

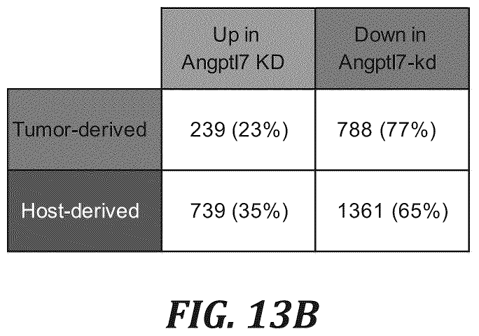

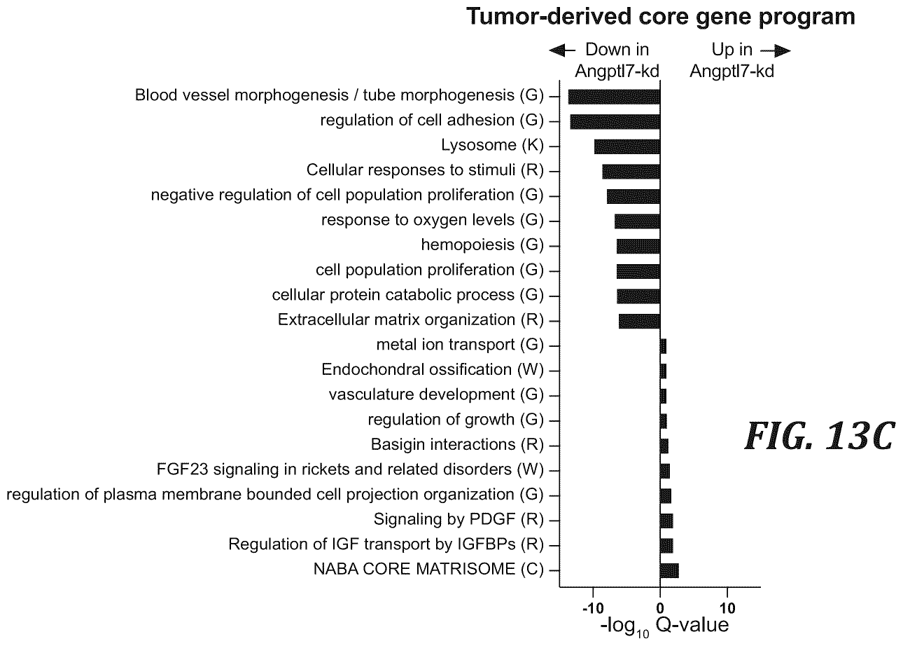

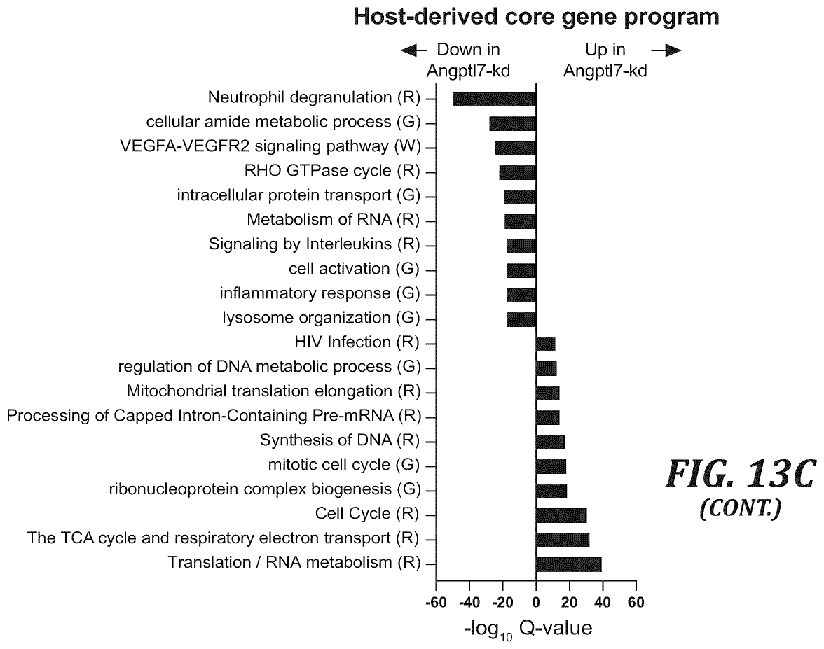

Given the marked decrease in intratumoral necrosis, CTC abundance, and metastasis when Angptl7 was suppressed, Angptl7 knockdown tumors were transcriptionally characterized. Bulk RNA-seq was performed, tumor core and rim were compared, and tumor and host-derived transcripts were deconvoluted. The inventors applied tumor core and rim gene sets from our initial xenograft deconvolution experiment (FIG. 2A), evaluated gene set enrichment between conditions, and observed marked depletion of tumor-derived core and host-derived core gene expression in both core and rim of Angptl7 knockdowns (FIG. 13A). Likewise, marked enrichment was observed for tumor-derived rim and host-derived rim gene expression in both core and rim of Angptl7 knockdowns.

While depletion of core gene expression was in line with the significant reduction in necrosis observed by H&E, this depletion was not complete, with 23% of tumor derived core genes and 35% of host derived core genes increased in Angptl7-knockdowns (FIG. 13B). Gene ontology analysis was performed to stratify the tumor core program into normalized and nonnormalized components. Tumor-derived blood vessel morphogenesis genes and host-derived neutrophil degranulation gene sets were depleted in Angptl7-knockdown conditions, while core matrisome (tumor), RNA translation (host), and TCA transport (host) persisted or increased in Angptl7-knockdown condition compared with nontargeting control (FIG. 13C). Taken together, these transcriptional studies reveal that Angptl7 regulates largescale remodeling of the tumor core. Angptl7 supports expression of a subprogram of genes expressed in the tumor core that are involved in blood vessel morphogenesis and neutrophil degranulation. However, Angptl7-knockdown does not correct tumor core defects in RNA and nutrient metabolism, indicative of persistent cellular stress in the tumor interior.

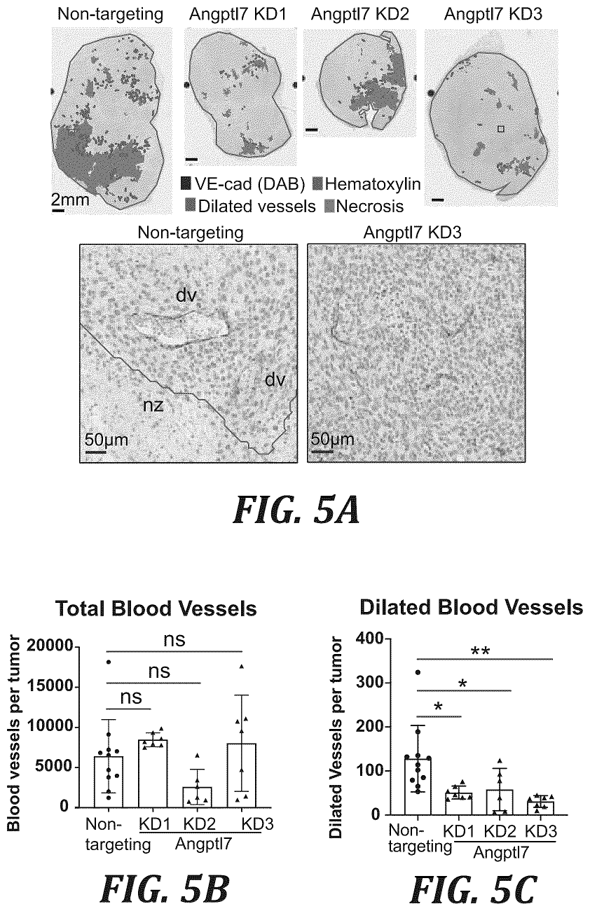

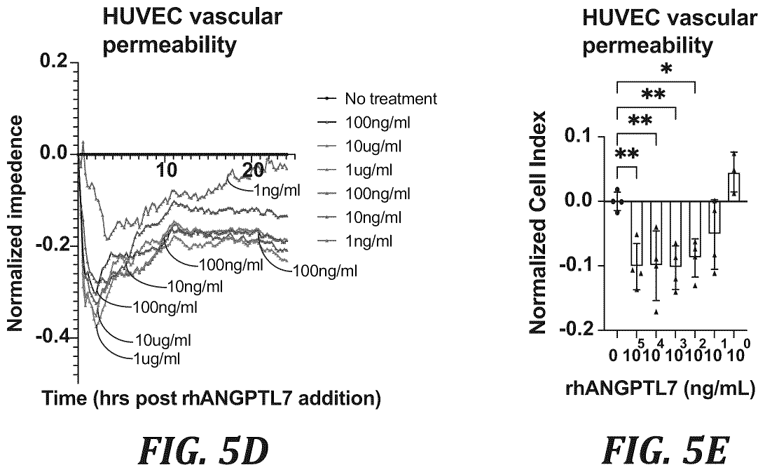

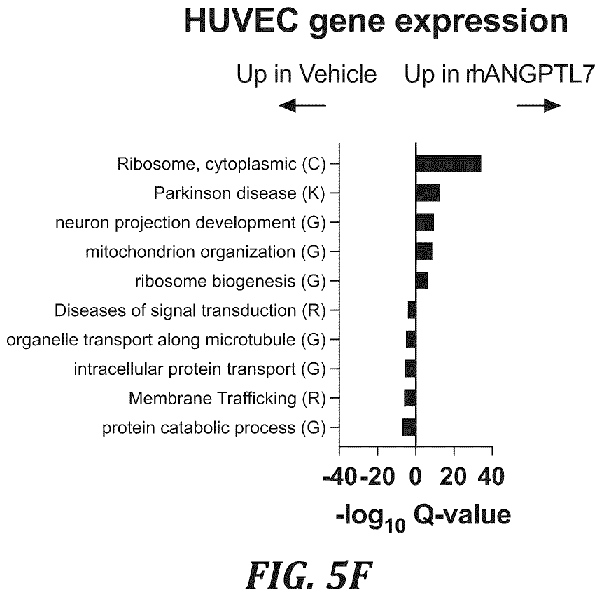

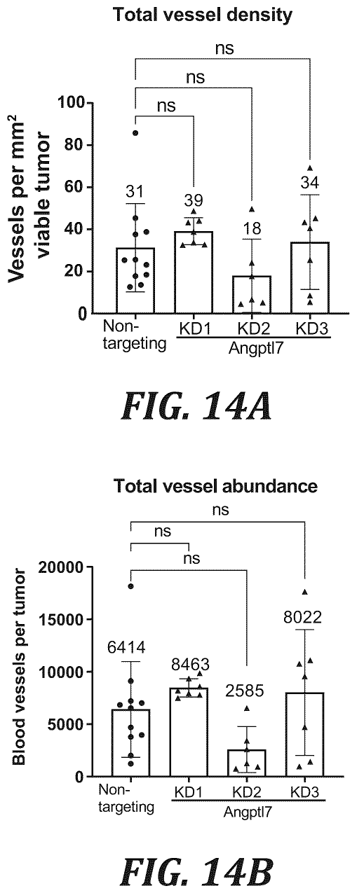

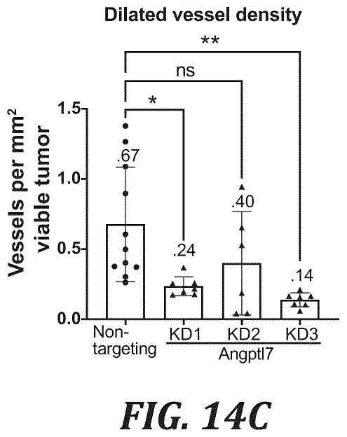

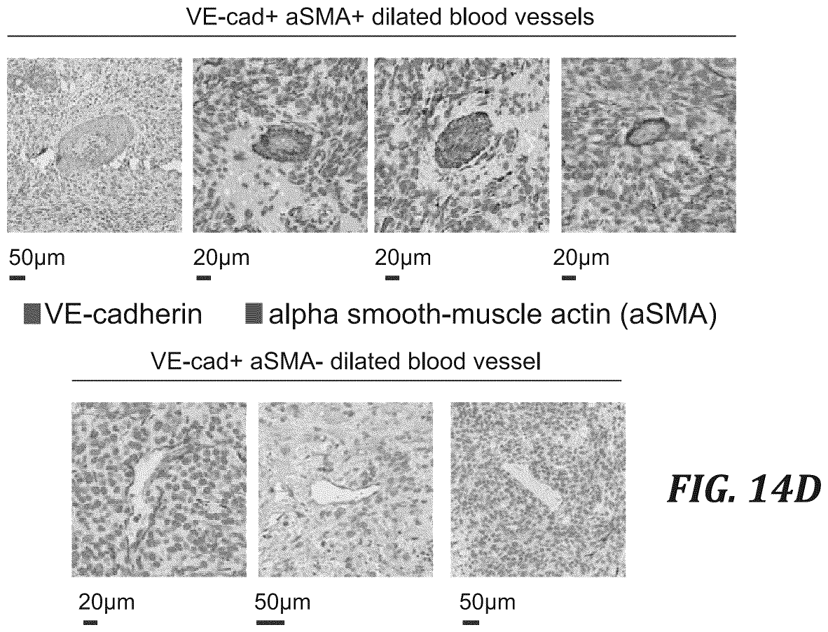

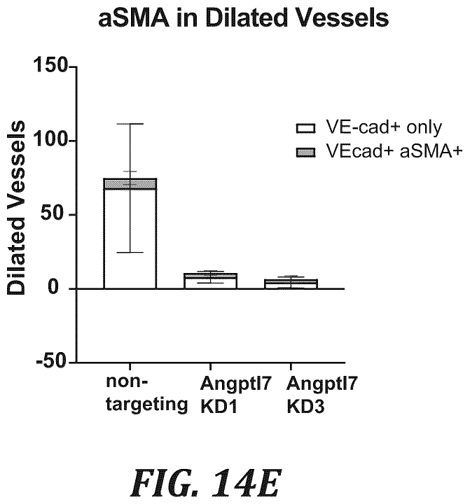

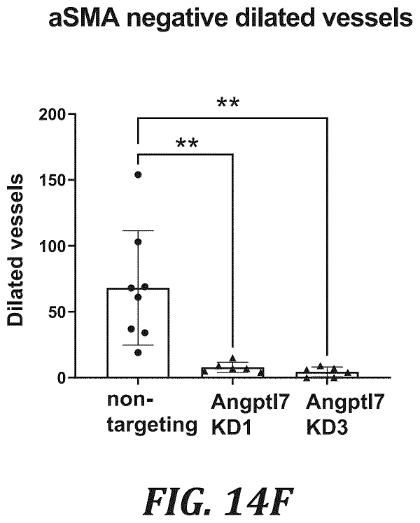

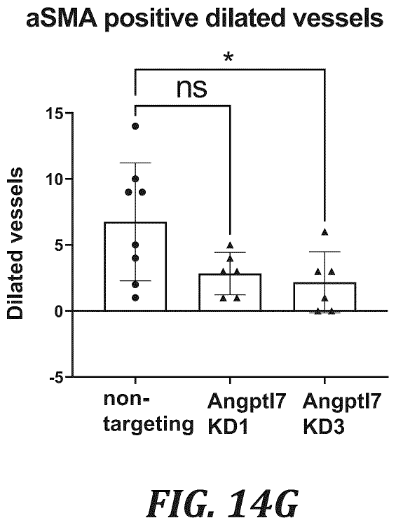

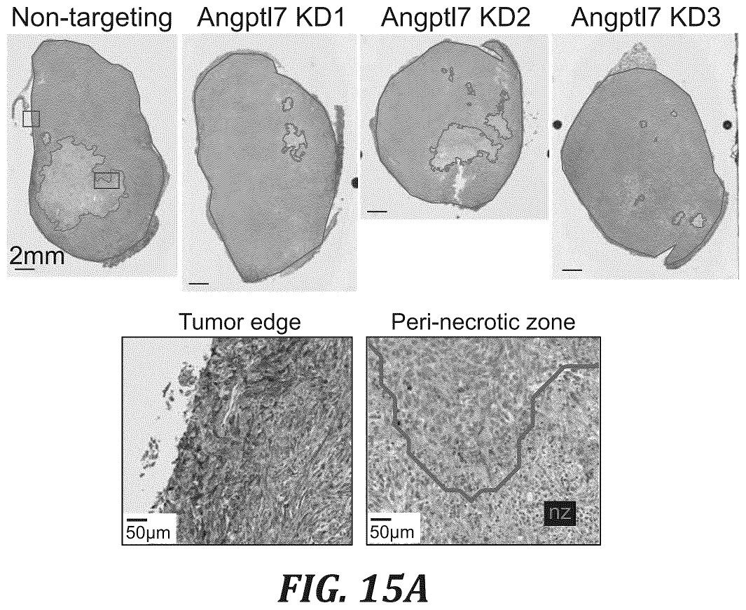

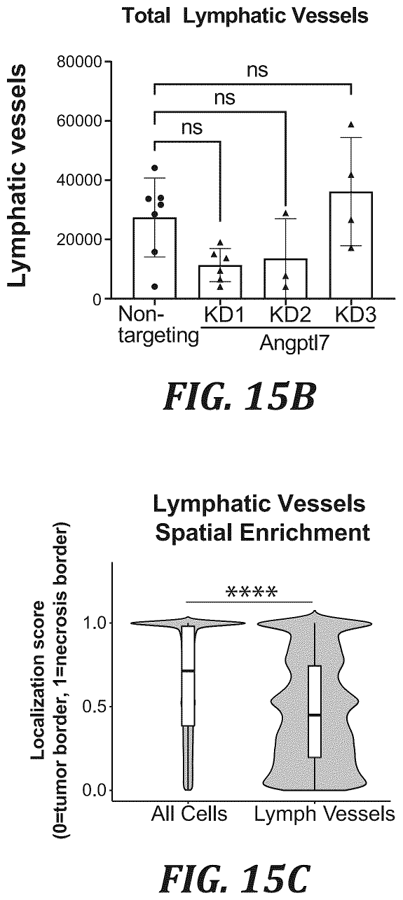

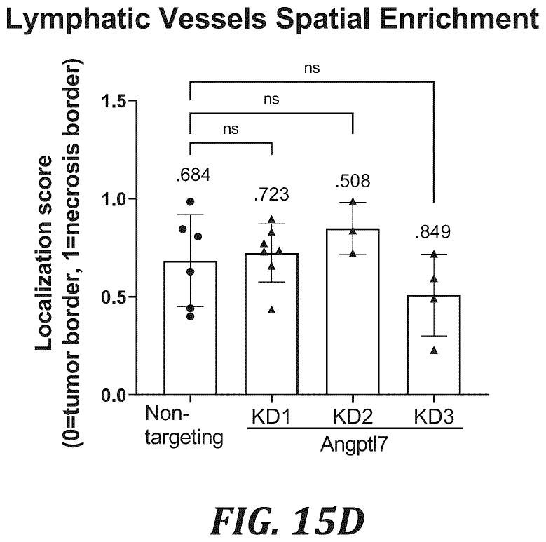

Having identified vascular morphogenesis genes as potentially Angptl7 regulated, the spatial distribution and expression of blood vessels in the Angptl7 knockdown tumors were next characterized (FIG. 5A). Total VE-cadherin-positive vessel number and density were similar between Angptl7 knockdown and nontargeting control tumors (FIG. 5B and FIGS. 14A and 14B). In contrast, the number and density of VE-cadherin-positive dilated vessels were markedly reduced in Angptl7 knockdown tumors (FIG. 5C and FIG. 14B). Further, smooth muscle actin staining, indicative of smooth muscle cell or contractile pericyte coverage, was uncommon around dilated vessels in both control and Angptl7 knockdown tumors, and not altered by Angptl7 suppression (FIGS. 14C-F). Because Angptl7 is also reported to regulate lymphangiogenesis, lymphatics were stained for podoplanin. In contrast to VE-cadherin, no significant difference in lymphatic vessel number, morphology, or perinecrotic localization was observed (FIGS. 15A-D). Lymphatic vessels were more prominent in viable regions of the tumor, closer to the tumor edge (FIGS. 15C and 15D). Taken together, these data show that Angptl7 is necessary for the perinecrotic dilated vessel phenotype.

Given the effect of Angptl7 suppression on dilated vessel morphology, the effect of ANGPTL7 was examined on vascular permeability. Human umbilical endothelial vein cells (HUVECs) plated on 2D were incubated with recombinant human ANGPTL7 from 1 ng/mL to 100 μg/mL and measured for changes in vascular permeability using a live-cell impedance assay. Strikingly, HUVECs showed a dose-dependent response to ANGPTL7 addition that was most pronounced by 1 h of treatment, which persisted out to 24 h at 10 ng/mL and higher recombinant ANGPTL7 concentrations (FIGS. 5D-F). To define gene pathways associated with ANGPTL7-induced changes, HUVEC cells were treated with 10 μg/mL ANGPTL7 for 24 h and then harvested for RNA-seq. Gene ontology analysis revealed dominant enrichment for cytoplasmic ribosome-encoding genes and weaker enrichment for genes involved in Parkinson disease, neuron projection development, and mitochondrial organization (FIG. 5F). Signaling pathway enrichment was observed for genes involved in SLIT and ROBO signaling (log Q-value−32), previously implicated in endothelial-tumoral crosstalk during metastatic dissemination (Tavora B, Mederer T, Wessel K J, et al. Tumoural activation of TLR3-SLIT2 axis in endothelium drives metastasis. Nature. 2020; 586(7828):299-304). Taken together, these data indicate that Angptl7 supports vascular remodeling in vivo, increases vascular permeability, and induces gene expression changes in endothelial cells in vitro.

ANGPTL7 is Highly Expressed in High-Necrosis Triple-Negative Human Breast Cancer Patient-Derived Xenografts (PDXs)

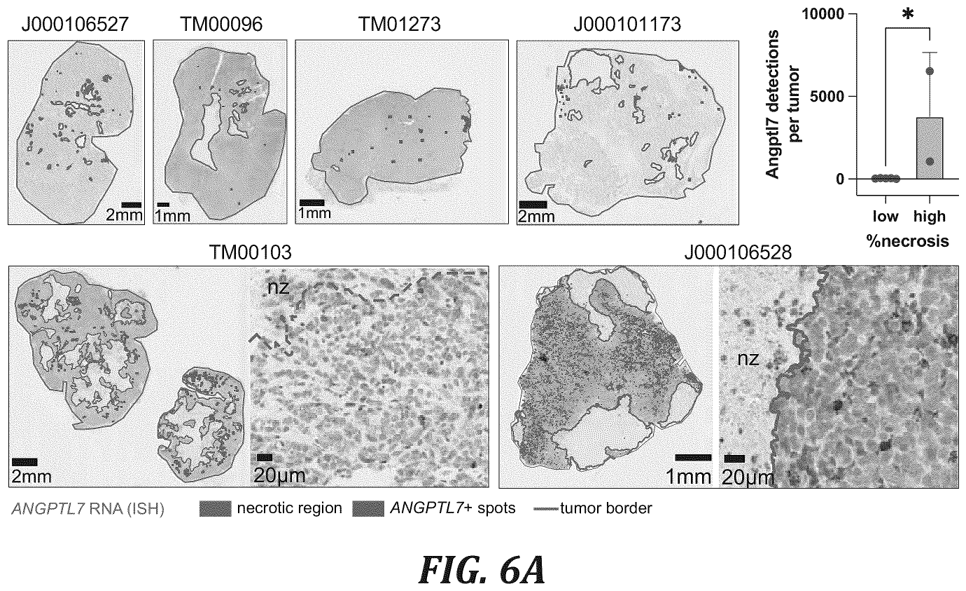

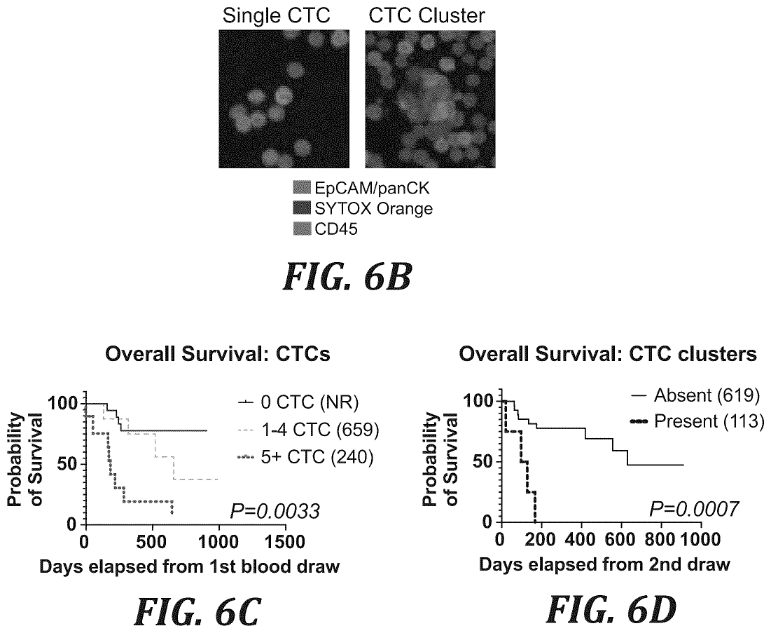

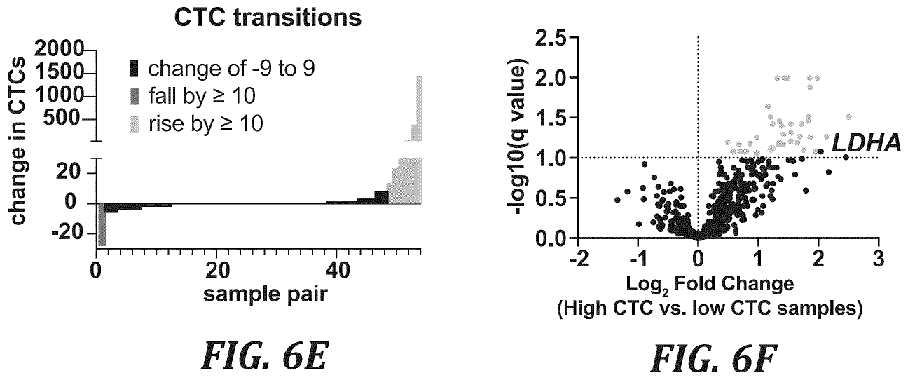

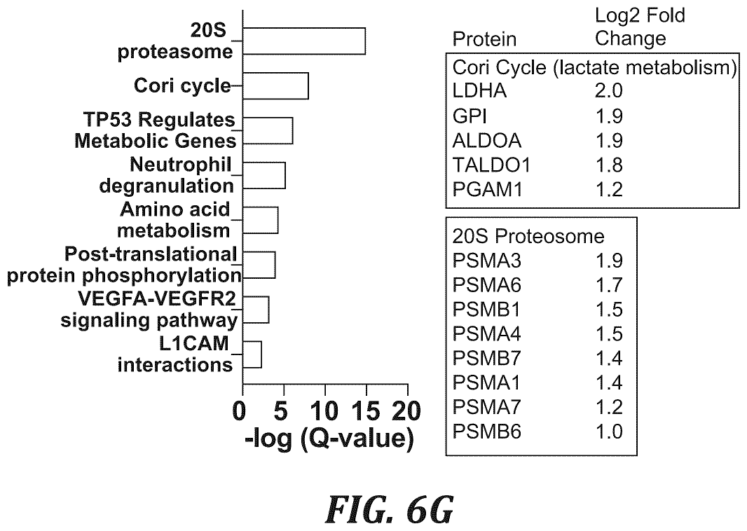

Clinically, necrotic zones are generally avoided during diagnostic tissue sampling due to lower abundance of viable tissue in these regions. To extend the human disease relevance of the findings in rat models, ANGPTL7 mRNA was evaluated by RNA-ISH from seven tumor sections from six different human breast cancer patient-derived xenografts (PDXs) each derived from high-grade invasive ductal carcinomas, and each triple-negative (ERnegative, PR-negative, HER2-negative receptor status) by PDX tumor histology (FIG. 6A). Even among these high-grade tumors, the percentage of necrosis varied markedly between models. Likewise, ANGPTL7 expression varied between models and was markedly higher in high necrosis models. For comparison, model J000106527, which had very low necrosis and ANGPTL7 expression was derived from a patient with T4bN0 high-grade triple-negative breast cancer (TNBC). This patient developed a second primary breast cancer 4 years later that was resected and then treated with adjuvant endocrine therapy. In contrast, model J000106528, which had the highest necrosis and ANGPTL7 expression of all models tested, was derived from a patient with T3N2 high-grade TNBC. This patient developed metastatic breast cancer within 2 years from initial diagnosis and tumor resection and progressed rapidly on therapy. Taken together, these clinical observations and experimental studies in PDX models demonstrate that ANGPTL7 is associated with TNBCs with large necrotic zones.

Necrosis Markers Are Associated with CTC Dissemination and Metastasis in Breast Cancer Patients

Given the association between necrosis and CTC abundance in the rat model, it was tested whether necrosis markers are associated with CTC dissemination in human breast cancer patients. To do this, longitudinal CTC abundance was interrogated from patients with metastatic breast cancer recruited to an IRB approved study at the Fred Hutchinson Cancer Center and the University of Washington (FH 8649). Over a 3-y period, a total of 102 blood samples from 40 patients were longitudinally collected including 35 women with estrogen receptor-positive metastatic tumors and five with estrogen receptor status that was negative or unknown. For each blood sample, the cellular fraction was separated by density, deposited to slides, and enumerated for CTCs by computer-assisted image analysis using the Rarecyte platform. Twenty-nine patients (73%) had at least one CTC at any timepoint, five patients (13%) had at least one CTC cluster at any timepoint, and 11 patients (28%) had no CTCs at any timepoints. As expected, based on prior studies, patients with CTCs detected at first blood draw had significantly worse overall survival and early death (FIGS. 6 B-C). Likewise, patients with CTC clusters at first or second blood draw had markedly worse overall survival and early death (FIGS. 6 B and D).

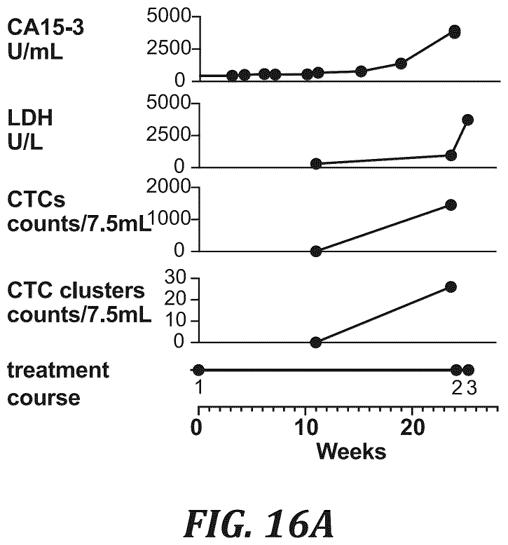

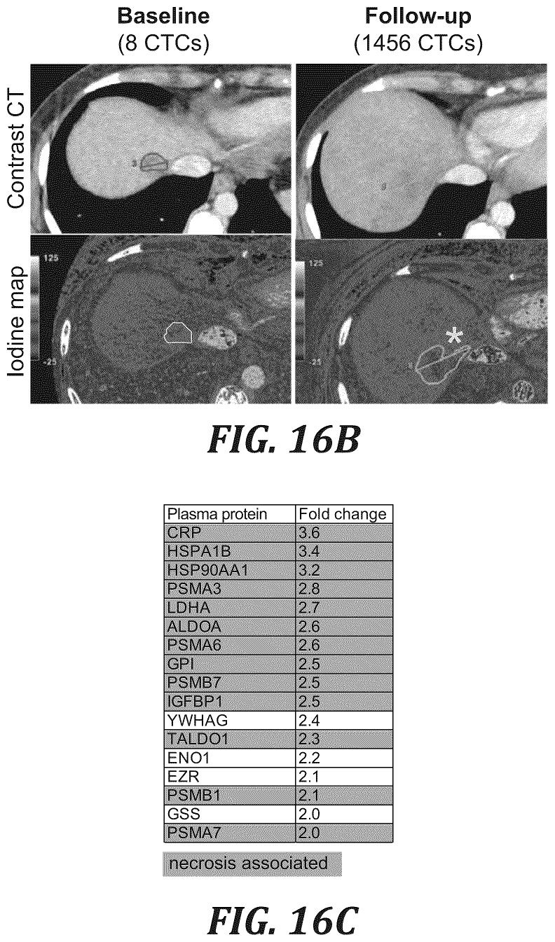

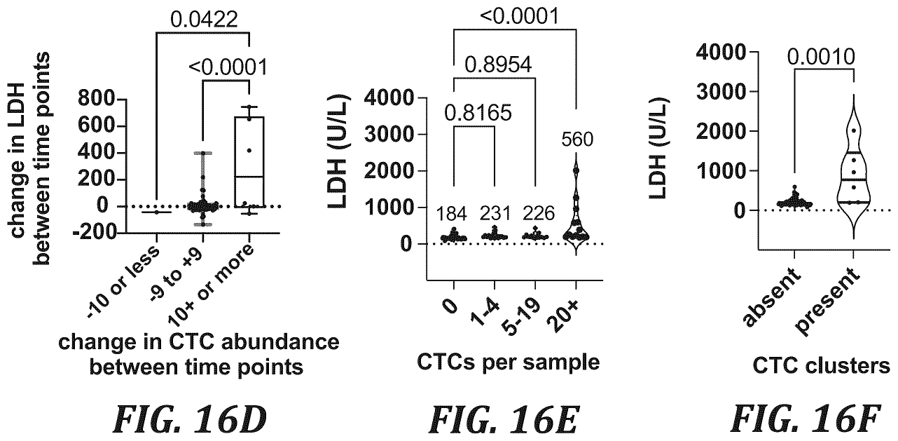

Among patients with CTCs at any time point, substantial differences in CTC dynamics were observed, and in a subset of cases, extreme elevations in CTC abundance between time points (FIG. 6E and FIGS. 16A and 16B for case vignette). Sixteen blood samples were selected from eight patients, made up of matched early and late time-points. Blood plasma from all 16 samples were depleted of high-abundance proteins, labeled with tandem-mass-tags, and interrogated by LC-ESI mass spectrometry. Comparison of high-CTC samples with all low-CTC samples yielded 46 proteins highly associated with high-CTC state and passing q-values of 0.1 or less (FIG. 6F). ANGPTL7 was not detected by LC-ESI mass spectrometry precluding its comparison between CTC states. Metascape gene set analysis demonstrated the top enrichment being for gene sets linked to necrosis (FIG. 6G) including: 1) Cori cycle, the major pathway for lactate metabolism, 2) the 20S proteosome which has been associated with necrosis, and 3) neutrophil degranulation, which was identified as a top gene set in the inventors' core/rim RNA-seq analyses (de Martino M, Hoetzenecker K, Ankersmit H J, et al. Serum 20S proteasome is elevated in patients with renal cell carcinoma and associated with poor prognosis. British Journal of Cancer 2012 106:5. 2012; 106(5):904-908; Lavabre-Bertrand T, Henry L, Carillo S, et al. Plasma Proteasome Level Is a Potential Marker in Patients with Solid Tumors and Hemopoietic Malignancies. Published online 2001). To control interpatient variation, a pair-wise comparison of late vs. early time points was conducted from three patients with low-to-high CTC transitions. Of the 17 proteins increased twofold or more between late and early time points, 13 (76%) were necrosis associated (FIG. 16C). To validate our proteomic observations, levels of the necrosis associated marker lactate dehydrogenase (LDH) was measured from plasma for 93 blood samples from 39 patients. Consistent with the inventors' proteomic enrichments, marked elevations in LDH were associated with a 10 or more increase in CTC abundance between time points, with blood samples with 20 or more CTCs, and with blood samples with CTC-clusters (FIGS. 16D-F). Taken together, these clinical, biochemical, and proteomic studies show that as seen in the rat model experiments, low-to-high CTC transitions are associated with increasing CTCs and markers of necrosis in patients with metastatic breast cancer.

Discussion

A key challenge in metastasis research is to discern where and how tumor cells disseminate to distant organs. However, understanding where metastatic dissemination originates from is a formidable challenge. Tumor dissemination is a dynamic process that is difficult to capture, and occurring in the context of spatially heterogeneous ecosystems varying in their nutrient availability, perfusion, oxygenation, and host contributions. This Example describes a rat transplantation model of breast cancer that increases CTC detection 10-fold and leveraged this model to identify cellular and molecular changes in primary tumor associated with tumor dissemination. Importantly, tumor dissemination was strongly correlated temporally with necrosis in animal models and in human cancer patients, that tumor dissemination was localized spatially to dilated perinecrotic vessels in the tumor interior, and that tumor dissemination was dependent functionally on the expression of a factor, Angptl7, produced by perinecrotic tumor cells. The findings disclosed in this Example for breast cancer models, in conjunction with recent clinical observations (Lee G, Yoon S, Ahn B, Kim H R, Jang S J, Hwang H S. Blood Vessel Invasion Predicts Postoperative Survival Outcomes and Systemic Recurrence Regardless of Location or Blood Vessel Type in Patients with Lung Adenocarcinoma. Annals of Surgical Oncology. 2021; 28(12):7279-7290; Zhao Y, Fu X, Lopez J I, et al. Selection of metastasis competent subclones in the tumour interior. Nat Ecol Evol. 2021; 5(7):1033. doi:10.1038/S41559-021-01456-6), provide strong evidence for tumor dissemination from the tumor interior.

Decoding the molecular regulation of dissemination in the tumor interior is a particularly challenging chicken-and-egg problem because the tumor core is composed of regions that are hypoxic, deficient in nutrients, and abnormally perfused, each of which could influence and in turn be influenced by adaptive responses from tumor cells in the core. The very large tumors produced in the rat model enable spatial dissecting of the tumor core from tumor rim for transcriptional profiling, and the use of xenograft deconvolution methods further enabled discernment of which genes were expressed specifically in tumor and host. In this regard, the use of a mouse-in-rat xenograft was serendipitous and essential. The results disclosed in this Example suggest that rats are a valuable model organism with unique attributes supporting their use in metastasis research alongside more commonly used in vivo models such as mouse and zebrafish. These studies unmasked a tumor-derived program in the core and identified Angptl7 as the top-ranked secreted factor. Strikingly, Angptl7 suppression normalizes histologic findings of central necrosis and dilated perinecrotic vasculature. On a molecular level, Angptl7 depletion resolves vascular morphogenetic, hypoxic, and inflammatory changes associated with the tumor core but not gene programs associated with stress, RNA metabolism and nutrient depletion. These findings suggest that tumor necrosis arises from the sequential activation of stress programs in the core that induce a tumor-cell-driven transcriptional response including Angptl7. Thus, Angptl7 is both regulated by the local microenvironment in the tumor interior and a regulator of further central necrosis and metastatic dissemination. Future studies are needed to define the signals and regulatory programs driving the tumor core adaptive response program.

Previous studies have shown that tumor dissemination can depend on macrophage-led migration, vascular mimicry, EMT, and collective migration (Yamamoto A, Doak A E, Cheung K J. Orchestration of Collective Migration and Metastasis by Tumor Cell Clusters. Annual Reviews Pathology. 2023; 18:231-256; Wrenn E, Huang Y, Cheung K. Collective metastasis: coordinating the multicellular voyage. Clinical and Experimental Metastasis. 2021; 38(4):373-399; Beerling E, Seinstra D, de Wit E, et al. Plasticity between Epithelial and Mesenchymal States Unlinks EMT from Metastasis-Enhancing Stem Cell Capacity. Cell Reports. 2016; 14(10):2281-2288; Harney A S, Arwert E N, Entenberg D, et al. Real-Time Imaging Reveals Local, Transient Vascular Permeability, and Tumor Cell Intravasation Stimulated by TIE2hi Macrophage-Derived VEGFA. Cancer Discovery. 2015; 5(9):932-943; Silvestri V L, Henriet E, Linville R M, Wong A D, Searson P C, Ewald A J. A Tissue-Engineered 3D Microvessel Model Reveals the Dynamics of Mosaic Vessel Formation in Breast Cancer. Cancer Research. 2020; 80(19):4288-4301; Wagenblast E, Soto M, Gutiérrez-Ángel S, et al. A model of breast cancer heterogeneity reveals vascular mimicry as a driver of metastasis. Nature 2015 520:7547. 2015; 520(7547):358-362; Maniotis A J, Folberg R, Hess A, et al. Vascular channel formation by human melanoma cells in vivo and in vitro: vasculogenic mimicry. American Journal of Pathology. 1999; 155(3):739-752; Cheung K J, Gabrielson E, Werb Z, Ewald A J. Collective invasion in breast cancer requires a conserved basal epithelial program. Cell. 2013; 155(7):1639-1651; Cheung K J, Padmanaban V, Silvestri V, et al. Polyclonal breast cancer metastases arise from collective dissemination of keratin 14-expressing tumor cell clusters. Proceedings of the National Academy of Sciences U S A. 2016; 113(7):E854-E863; Gkountela S, Castro-Giner F, Szczerba B M, et al. Circulating Tumor Cell Clustering Shapes DNA Methylation to Enable Metastasis Seeding. Cell. 2019; 176(1-2):98-112.e14; Liu X, Taftaf R, Kawaguchi M, et al. Homophilic CD44 Interactions Mediate Tumor Cell Aggregation and Polyclonal Metastasis in Patient-Derived Breast Cancer Models. Cancer Discovery. 2019; 9(1):96-113). The data disclosed in this Example establish the importance of the necrotic zone in supporting metastatic dissemination and highlight the presence of intravascular tumor emboli within dilated perinecrotic vessels. This Example discloses that both dilated vessels and Angptl7 were on average 2 mm away from the nearest tumor border. Given the great distance of the central necrotic zone to the tumor-stromal interface, advances in intravital imaging in deep tissues (Bakker G J, Weischer S, Ortas J F, et al. Intravital deep-tumor single-beam 3-photon, 4-photon, and harmonic microscopy. Elife. 2022; 11; Scheele C L G J, Herrmann D, Yamashita E, et al. Multiphoton intravital microscopy of rodents. Nature Reviews Methods Primers 2022 2:1. 2022;2(1):1-26) will be essential to evaluate which of these cellular dissemination mechanisms predominates in the necrotic zone. In addition, organotypic culture models have proven useful in modeling invasion in vitro that have morphologic correlates in vivo (Cheung K J, Gabrielson E, Werb Z, Ewald A J. Collective invasion in breast cancer requires a conserved basal epithelial program. Cell. 2013; 155(7):1639-1651; Cheung K J, Padmanaban V, Silvestri V, et al. Polyclonal breast cancer metastases arise from collective dissemination of keratin 14-expressing tumor cell clusters. Proceedings of the National Academy of Sciences U S A. 2016; 113(7):E854-E863; Gaggioli C, Hooper S, Hidalgo-Carcedo C, et al. Fibroblast-led collective invasion of carcinoma cells with differing roles for RhoGTPases in leading and following cells. Nature Cell Biology. 2007; 9(12):1392-1400; Wrenn E D, Yamamoto A, Moore B M, et al. Regulation of Collective Metastasis by Nanolumenal Signaling. Cell. 2020; 183(2):395). Transcriptional dissection provides a reference point in which to develop organotypic models that more faithfully mimic the necrotic core-associated dissemination program. In particular, the formation of dilated vessels is strongly associated with tumor dissemination and metastasis. At present, how vessels become dilated is unclear. Whether dilated vessels arise from extrinsic compression from proliferating tumor cells, influence of the extracellular matrix, or reflects occlusion by tumor cell emboli is unclear and could benefit from better organotypic models incorporating vasculature. Finally, previous clinical and experimental studies have shown that tumors can disseminate early preceding the detection of frank invasive cancer (Hu Z, Curtis C. Looking backward in time to define the chronology of metastasis. Nature Communications 2020 11:1. 2020; 11(1):1-4; Klein C A. Cancer progression and the invisible phase of metastatic colonization. Nature Reviews Cancer. 2020; 20(11):681-694), while at the same time, that very large tumors are more likely to develop metastatic cancer (Heimann R, Hellman S. Clinical progression of breast cancer malignant behavior: what to expect and when to expect it. Journal of Clinical Oncology. 2000; 18(3):591-599; Minn A J, Gupta G P, Padua D, et al. Lung metastasis genes couple breast tumor size and metastatic spread. Proceedings of the National Academy of Sciences U S A. 2007; 104(16): 6740). The data disclosed in this Example are compatible with these clinical observations. Because tumor necrosis can arise independently of tumor invasion, small fast-growing tumors could undergo necrosis early, while large tumors could undergo necrosis late. Consistent with the inventors' experimental observations, comedonecrosis is associated with increased risk of death from in situ, preinvasive cancers (Narod S A, Iqbal J, Giannakeas V, Sopik V, Sun P. Breast Cancer Mortality After a Diagnosis of Ductal Carcinoma In Situ. JAMA Oncology. 2015; 1(7):888-896).

From a therapeutic standpoint, the data disclosed in this Example indicate that ANGPTL7 is a fulcrum for eliciting central necrosis and metastatic dissemination. ANGPTL proteins have three conserved domains including an N-terminal coiled-coil domain that mediates homo-oligomerization, a linker peptide, and a C-terminal fibrinogen-like domain (Carbone C, Piro G, Merz V, et al. Angiopoietin-Like Proteins in Angiogenesis, Inflammation and Cancer. International Journal of Molecular Sciences. 2018; 19(2)). More studies are needed to investigate the functional domains of ANGPTL7 necessary for necrosis and metastatic dissemination. Therapeutic blocking antibodies and anti-sense oligonucleotides targeting ANGPTL3 reduce atherogenic lipoprotein levels and decrease the odds of developing cardiovascular disease in humans (Dewey F E, Gusarova V, Dunbar R L, et al. Genetic and Pharmacologic Inactivation of ANGPTL3 and Cardiovascular Disease. New England Journal of Medicine. 2017; 377(3): 211-221; Graham M J, Lee R G, Brandt T A, et al. Cardiovascular and Metabolic Effects of ANGPTL3 Antisense Oligonucleotides. New England Journal of Medicine. 2017; 377(3): 222-232), supporting ANGPTL family of proteins as druggable targets. Necrosis is associated with aggressive tumors and is a common tumor response to many cancer treatments. Given the pronounced effects of Angptl7 depletion in the rat model, the date from this Example suggest that ANGPTL7 is a target for future drug development for necrosis and metastasis suppression.

Materials and Methods

Experimental Model and Subject Details

Animal models. All mice were maintained under specific pathogen-free conditions, and experiments conformed to the guidelines as approved by the Institutional Animal Care and Use Committee of Fred Hutchinson Cancer Research Center (FHCC).). The SRG OncoRats, SCID rats on the Sprague-Dawley background that harbors a double knockout for the Rag2 and Il2rgamma genes (SRG), were purchased from Hera Biolabs. Similar to NSG mice, SRG rats are a double knockout for the Rag2 and Il2rgamma genes (SDRag2tm2hera Il2rgtm1hera) and lack B-cells, T-cells, and NK-cells. For mouse experiments, NSG mice (NOD.Cg-Prkdcscid Il2rgtm1Wjl/SzJ) were used. 8-13-week-old female mice and rats were used for all experiments.

Human breast cancer patient samples. Blood from patients were obtained from consenting patients under a Fred Hutch IRB approved study (FH8649) for longitudinal monitoring of circulating tumor cells in metastatic breast cancer patients. CTCs and CTC-clusters were enumerated in these fluid samples using a RareCyte assay (Kaldjian E P, Ramirez A B, Sun Y, et al. The RareCyte® platform for next-generation analysis of circulating tumor cells. Cytometry. 2018; 93(12):1220).

Cell Lines

293FT (ThermoFisher Scientific R70007) cell line was grown at 37° C., 5% CO2 in DMEM high glucose, GlutaMAX supplement, pyruvate (GIBCO 10569-010) supplemented with 10% fetal bovine serum (Sigma-Aldrich F0926-500ML) and 1% penicillin/streptomycin (Sigma-Aldrich P4333). 4T1 (ATCC CRL-2539™) and HCC70 (ATCC CRL2315) cell line was grown at 37° C., 5% CO2 in RPMI, GlutaMAX supplement (GIBCO 61870-127) supplemented with 10% fetal bovine serum (Sigma-Aldrich F0926-500ML) and 1% penicillin/streptomycin (Sigma-Aldrich P4333). For non-adherent culture, cell lines were cultured in complete media+2% (v/v) Matrigel. All cell lines used were from human females. 4T1 Cells were purchased from American Type Culture Collection (ATCC). 4T1-cyto-eGFP cells were a gift from the Cyrus Ghajar at Fred Hutchinson Cancer Center. 4T1-AcGFP1-mem-9 cells were generated by transducing 4T1 cells from ATCC with the rLV.EF1.AcGFP1-Mem-9 lentiviral particle (MOI=5) (Takara Bio). 4T1-AcGFP1-mem-9 cells were selected with 10 ug/ml puromycin for at least 2 weeks.

Methods

Generating knockdown 4T1 cell lines. 4T1-AcGFP1-mem-9 cells were transduced with lentiviral particles generated with shERWOOD UltramiR Lentiviral Inducible shRNA (TransOMIC) for Angptl7 and selected with 10 ug/mL puromycin+10 ug/mL blasticidin for at least 2 weeks.

Orthotopic transplantation into mammary fat pad of rats and mice. Cells to be transplanted were cultured 3D for two passages before being transplanted. After the second passage, cells were plated in nonadherent six-well plates at 150 k cells/mL with 4 mL media per well for the cells to aggregate over 24 h before being transplanted. Spheroids formed this way were resuspended in 1:1 Matrigel:DMEM/F12 mix, kept on ice. A total of 600,000 cells in 20 μL Matrigel DMEM/F12 mix were transplanted into the right T4 mammary fat pad of rats or mice. Estimated tumor volume was calculated based on caliper measurements with the formula: V=(W2×L)/2.

Density separation of coat (nucleated cells) from blood. Peripheral blood was diluted 1:1 with D-PBS then layered on top of 8 mL Ficoll (GE Ficoll Paque Plus GE17-1440-02 for human blood and Ficoll Paque Premium GE17-5442-02 for mouse and rat blood) in a 2.5% BSA-coated 50 mL conical tube. Tubes with Ficoll and diluted blood were centrifuged at 400 g for 35 min with 0 acceleration and deceleration. The cloudy, white layer (“buffy coat”) between the clear Ficoll layer and the top plasma layer and the plasma layer were collected into a separate BSA-coated tube. Buffy coat and plasma layer mix were centrifuged at 4° C. at 3,500 g for 30 min to pellet nucleated cells, then resuspended with D-PBS. To visualize Buffy coat containing CTCs, cell suspension was spun onto slides using a Cytospin (800 g for 5 min). After drying slides completely, slides were fixed with 4% PFA for 5 min, washed with D-PBS 5 min three times. Slides were dried completely again before being stored in ˜80° C. For plasma collection, plasma layer was collected from the plasma layer from the Ficoll density separation or from a separate blood tube in an AccuCyte Blood collection tube processed by Rarecyte with a 3,000 g 25 min spin at 25° C.

RNA-seq computational deconvolution of mouse and rat genomes. RNA-seq reads in xenograft samples were deconvolved for identification of the species of origin following a method similar to that described by Wingrove et al. (Minn A J, Gupta G P, Padua D, et al. Lung metastasis genes couple breast tumor size and metastatic spread. Proceedings of the National Academy of Sciences U S A. 2007; 104(16):6740).

QuPath quantification of RNA ISH. RNAscope images were opened in QuPath (Bankhead P, Loughrey M B, Fernández J A, et al. QuPath: Open source software for digital pathology image analysis. Scientific Reports. 2017; 7(1)). Thresholds were created to identify tumor area and necrotic area by eye, verified to be accurate in test images, and applied to demarcate necrotic zones. Angptl7-expressing cells were identified using positive cell detection. The distance to annotation 2d feature was used to determine the distance between cells and the nearest necrotic border and cells and nearest tumor border. This process was automated in QuPath for consistent analysis across images.

Necrosis measurements. Tumor necrosis measurements were determined based on hematoxylin & eosin (H&E) or 2,3,5-triphenyltetrazolium chloride (TTC) assay. For H&E staining, tumors were sliced into two to three ˜5 mm slices by scalpel, fixed for 5 d in 10% formalin at 4° C., rocking before being sectioned, and stained with hematoxylin and eosin. For the TTC assay, tumors were sliced into two to three ˜5 mm slices and stained with 1 g/100 mL tetrazolium salt in a 7.4 pH buffer with 77.4% NaH2PO4 (0.1 M) and 22.6% Na2HPO4 (0.1 M) mix, at 37° C. for 20 min, rocking. Tumor slices were then fixed with 10% formalin for 20 min before being visualized. In the TTC assay, the TTC compound is reduced to a red TPF (1,3,5-triphenylformazan) compound in live tissues due to dehydrogenase activity. White areas therefore indicate necrotic tissue, and red areas are viable regions.

2D Cell Culture. 4T1 cells were cultured 2D on adherent plates in RPMI +10% FBS+1% Penicillin-Streptomycin solution. 4T1-AcGFP1-mem-9 were cultured with RPMI complete +10 ug/ml puromycin. Angptl7 knockdowns and non-targeting control 4T1 cells were cultured with RPMI complete+10 ug/ml puromycin+10 ug/ml blasticidin.

3D Cell Culture. 2D cultured cells were trypsinized with 0.25% trypsin, then quenched with complete RPMI. Cell suspension was centrifuged at 400 g for 5 mins, then resuspended in Accumax. Accumax suspension was place in 37° C. water bath for 30 mins, pipetting up and down every 10 minutes. After centrifuging for 5 min at 400 g, pellet was resuspended in complete RPMI+2% Corning® Matrigel® Growth Factor Reduced (GFR) Basement Membrane Matrix (354230) (Corning). Cells were plated in non-adherent 6 well plates at 150,000 cells/mL with 4 mL media per well.

SRG rats. The SRG OncoRats, SCID rats on the Sprague-Dawley background that harbors a double knockout for the Rag2 and Il2rgamma genes, were purchased from Hera Biolabs. SRG rats are a double knockout for the Rag2 and Il2rgamma genes (SDRag2tm2hera Il2rgtm1hera) and lack B-cells, T-cells, and NK-cells.

Blood collection from rats and mice. For rats, peripheral blood was collected terminally though the left ventricle of the heart, into CellSearch CellSave tubes (Menarini-Silicon Biosystems) with heparin-coated syringe attached to an 18g needle. Rats were put under deep anesthesia with isoflurane to do this, and rats were euthanized immediately after blood collection. Blood tube was inverted at least 10 times immediately after blood collection, and blood was processed within 4 hours of collection. For mouse blood, peripheral blood of 8 mice were collected into one CellSave tube using a new heparin coated syringe for every mouse.

Immunofluorescence staining. Slides were thawed at room temp for 5 minutes. For OCT tissue sections, slides were washed 5 mins 3× to wash OCT off. Slides were block for 1 hour with blocking solution (2.5% BSA, 5% normal goat serum or normal donkey serum, 0.3% triton in D-PBS). Primary antibody in blocking solution was placed on slides and incubated at room temp for 2 hours. We then washed the slides mins 3× with D-PBS before placing the secondary antibody in 5% normal goat serum or normal donkey serum in D-PBS on the slide to incubate for 1 hour. Slide was washed again mins 3× with D-PBS. Excess moisture was removed from the slide, and slide was mounted with 30-50 μl Prolong Diamond Antifade Mountant and glass coverslip. Slide was cured overnight room temp, then stored at 4° C.

Bulk RNA-seq. 4T1 cells were transplanted into the right #4 mammary fat pad of SRG rats. At day 27 post-transplantation, primary tumors were harvested. Tumor was cut in half to expose the cross section. The inner, necrotic region and the outer, non-necrotic region were separated and snap frozen for RNA-seq and sent to Genewiz-Azenta for RNA extraction, library prep, and sequencing. Qiagen RNeasy kit (Qiagen, Inc 74104) was used for in-house RNA extraction before samples were sent to Genewiz.

Azenta Genewiz RNA-seq. Sample QC, library preparations, sequencing reactions, and initial bioinformatic analysis were conducted at GENEWIZ, LLC./Azenta US, Inc (South Plainfield, NJ, USA) as follows:

Sample QC:

Total RNA samples were quantified using Qubit 2.0 Fluorometer (Life Technologies,

Carlsbad, CA, USA) and RNA integrity was checked with 4200 TapeStation (Agilent Technologies, Palo Alto, CA, USA).

Library Preparation and Sequencing:

Samples were initially treated with TURBO DNase (Thermo Fisher Scientific, Waltham, MA, USA) to remove DNA contaminants. The next steps included performing rRNA depletion using QIAseq® FastSelect™-rRNA HMR kit (Qiagen, Germantown, MD, USA), which was conducted following the manufacturer's protocol. RNA sequencing libraries were constructed with the NEBNext Ultra II RNA Library Preparation Kit for Illumina by following the manufacturer's recommendations. Briefly, enriched RNAs are fragmented for 15 minutes at 94° C. First strand and second strand cDNA are subsequently synthesized. cDNA fragments are end repaired and adenylated at 3′ends, and universal adapters are ligated to cDNA fragments, followed by index addition and library enrichment with limited cycle PCR. Sequencing libraries were validated using the Agilent Tapestation 4200 (Agilent Technologies, Palo Alto, CA, USA), and quantified using Qubit 2.0 Fluorometer (ThermoFisher Scientific, Waltham, MA, USA) as well as by quantitative PCR (KAPA Biosystems, Wilmington, MA, USA). The sequencing libraries were multiplexed and clustered on one lane of a flowcell. After clustering, the flowcell was loaded on the Illumina HiSeq 4000 instrument according to manufacturer's instructions. The samples were sequenced using a 2×150 Pair-End (PE) configuration.

Initial Bioinformatics analysis:

Image analysis and base calling were conducted by the HiSeq Control Software (HCS). Raw sequence data (.bcl files) generated from Illumina HiSeq was converted into FASTQ files and de-multiplexed using Illumina's bcl2fastq 2.17 software. One mismatch was allowed for index sequence identification.

RNA-Seq Computational Deconvolution of Mouse and Rat Genomes:

RNA-seq reads in xenograft samples were deconvolved for identification of the species of origin following a method similar to that described by Wingrove et al.2. A catenated genome was formed by catenating the Rat (Rnor_6.0) and mouse (GRCmm28) genomes and gene annotations. Prior to alignment, reads were trimmed using Trim Galore and read quality was confirmed using fastQC. Trimmed reads were identified as originating from either rat or mouse by aligning reads against the catenated genome using STAR. Paired reads with both mates aligning uniquely to either the rat or mouse genome were separated by species and counted to gene annotations using featureCounts for downstream differential expression analysis. Within-species normalization of mapped reads and differential expression analysis were performed using the limma (version 3.50.0) and edgeR (version 3.36.0) packages in R following the procedure described in detail by Law et al.6. Prior to differential expression analysis, very low abundance genes were filtered using the filterByExpr function in edgeR using default parameters. Effective library sizes were normalized across conditions using the trimmed mean of M-values (TMM) normalization procedure. Differential expression analysis was performed on the filtered and normalized counts using the limma-voom linear modeling pipeline. For experiments in which samples were paired by animal, animal ID was treated as an additional random effect in the linear model. Empirical Bayes smoothing was applied on both the linear model and fitted contrast coefficients using edgeR. False-discovery rates for differential expression at the gene-level were calculated using the Benjamini-Hochberg correction procedure with the toptable function in limma. Normalized transcript abundances are reported as the TMM-normalized counts-per-million obtained from the voom function in limma. Gene set enrichment analysis was performed using GSEA software (Broad Institute, v4.2.3). Genes were ranked by log-fold change and collapsed to human orthologs using the MDSig ensembl gene id orthologs chip platform (v 7.3). GSEA was performed using a pre-ranked analysis weighted by log-fold change against the C5 gene ontology collection (v 7.5). False-discovery rates were determined using 100,000 permutations. For differential expression analysis between species (tumor and host), a modified normalization scheme was applied to account for differences in gene lengths and annotation depth between species, in addition to the standard normalization by effective library size. Our method resembles that described by Oziolor, et al. First, using reads that mapped uniquely to one species of the catenated genome, an intra-species TMM normalization was performed separately for host- and tumor-mapped reads. Length-corrected transcript abundances were calculated as reads per kilobase million (RPKM) in edgeR, and then converted to transcripts per million (TPM). Homolog pairs were obtained by querying the Ensembl database for linked rat (rattus norvegicus Ensembl release 100) and mouse (mus musculus Ensembl release 100) datasets using the getLDS function in biomaRt (v 2.50.3). Next, both gene sets were filtered to only those with rat/mouse homolog pairs, and the rat genes were mapped onto their corresponding mouse homologs. TPM abundances originating from both species were combined into a single data set, treating species of origin as a sample identifier, for all further analysis. To correct for differences in genome annotation depth between species, TPM values were normalized using the TMM method in edgeR. Homolog-wise differential expression analysis was performed between species using TMM-normalized, homolog-mapped TPM values as input into the limma-voom analysis pipeline (as above) in place of library-normalized counts. Prior to identifying top genes that were differentially expressed in the mouse tumor over the rat host, homolog pairs that exhibited zero read counts in the rat homolog for all of the samples were discarded, since these may reflect a limitation of the homolog mapping. Then, genes from mouse-core-mouse-rim were cross referenced with mouse-core-rat-core. Genes that were mouse-core vs. mouse-rim Log Fold Change≥1 and FDR≤0.01 and with detectable rat expression are shown in FIG. 4C.

Real Time qPCR. RNA from tumors and cell pellets were extracted using Qiagen RNeasy kit. RNA was reverse transcribed into cDNA using Superscript™ III First-Strand Synthesis System (Thermo Fisher Scientific Catalog number: 18080051) with equivalent amounts of RNA (1 μg). cDNAs were mixed with indicated gene-specific primers listed and PowerUp SYBR Green Master Mix (Thermo Fisher Scientific Catalog number: A25741) and qRTPCR was performed using an Applied Biosystems QuantStudio5 System.

Immunohistochemistry (IHC). Tumors were sliced into ˜5 mm sections immediately after harvest, placed into histology cassettes (#15181701A), and fixed with 10% neutral buffered formalin solution (#HT501128) at room temperature for 5 days. Cassetted tumor sections were paraffin processed, embedded, and cut by Experimental Histopathology Core at Fred Hutchinson Cancer Research Center. Paraffin-embedded tumor slides were warmed in 56° C. incubator for 20 min in slide holder. Tissue was deparaffinized with Histo-Clear solution (#64111-04) twice for min each. Rehydration was performed by immersing the slides through the following solutions twice for 3 min each: 100% ethanol, 95% ethanol, 70% ethanol, and deionized water. Heat-induced antigen retrieval was completed by incubating slides in pressure cooker at 6 psi (˜105° C.) for 10-15 min with Tris-EDTA (10 mM Tris Base, 1 mM EDTA, 0.05% Tween20, pH9.0) or sodium citrate (10 mM sodium citrate, 0.05% Tween20, pH6.0) buffers. Endogenous peroxidase activity was blocked by 3% H2O2 in PBS for 20 min. Fc receptor was blocked by Fc Receptor Blocker (#NB309) for 30 min. Primary antibodies were diluted with blocking solution (2.5% host serum/1.25% BSA in PBS) and incubated on the slides at room temperature for 1 hour. Species specific ImmPRESS™ secondary antibodies [HRP polymer] from Vector Laboratories were applied directly onto the slides and incubated for 30 min at room temperature. Substrate was developed using ImmPACT™ NovaRED™ HRP Substrate Kit (#SK-4805). Mayer's Hematoxylin (#26043-05) was used for counterstaining. Dehydration was performed by dipping the slides 10 times through the following solutions twice: 95% Ethanol and 100% Ethanol, followed by immersing the slides in Histo-clear solution twice for 2 min each. CytoSeal 60 (#18006) was used to mount slides, then slides were scanned by Ventana DP 200 slide scanner.

Western Blotting. Snap frozen tumors were milled into powder and 0.1 g was dispensed to make lysate. Lysis buffer was prepared by diluting protease (#87785) and phosphatase (#78420) cocktail inhibitors 1:100 in 1×RIPA buffer (#9806S) and added into tumor powder (1 mL of lysis buffer/0.1 g of powder). The suspension was sonicated for 6 pulses with a probe sonicator (20% duty cycle, 30% amplitude), incubated at 4° C. for 1 hour, and centrifuged at 14,000 g for 10 min to clear out insoluble substance. Protein concentration was quantified using Pierce BCA Assay Kit (#PI23225). Sample loading solution was prepared by combining tumor lysates (volume for 50 ug of protein), 3.3 uL of 10×Bolt reducing agent (#B0009), 8.25 uL of 4×Bolt LDS buffer (#B0007), and MiliQ water to a final volume of 33 uL. A Bolt™ 4-12%, Bis-Tris, 12-well, 1.0 mm Mini Protein Gel, (#NW04122BOX) was loaded with 30 uL of sample loading solution per well and run at 100V for 20 min and then at 170V for 40 min using 1×Bolt MES SDS running buffer (#B0002). The protein transfer sandwich was assembled using Immobilon-FL PVDF Membrane (#IPFL07810) and run at 15V for 1 hour with 1×Bolt transfer buffer (#BT00061) containing 10% methanol. The PVDF membrane was blocked with 3% BSA in TBS for 1 hour. Primary antibodies were diluted with 0.2% TBST and incubated overnight at 4° C. Species specific LI-COR 680 and 800 secondary antibodies were diluted 1:10,000 with 0.2% TBST plus 0.01% SDS and incubated for 1 hour at room temperature. The PVDF membrane was imaged with LiCor Odyssey CLx.

Angptl7 ELISA. Sample loading solution was prepared by combining tumor lysate (volume for 300 ug of protein) and sample diluent from the Mouse Angptl7 ELISA Kit ((#MBS612413) to a final volume of 400 uL. 100 uL of sample loading solution was loaded in each well for three replicates. Each well was incubated with biotinylated Anti-Mouse Angptl7 antibody, followed by Avidin-Biotin-Peroxidase Complex, and then Color Developing Reagent followed by Stop Solution per manufacturer instructions. O.D. absorbance was measured using a microplate reader at 450 nm.

Angptl7 RNA ISH. For in situ hybridization (ISH), formalin-fixed paraffin-embedded tissues were sectioned at 4 microns onto positively-charged slides and baked for 1 hour at 60° C. The slides were then dewaxed and stained on a Leica BOND Rx stainer (Leica, Buffalo Grove, IL) using Leica Bond reagents for dewaxing (Dewax Solution), antigen retrieval with Epitope Retrieval Solution 2 for 15 minutes at 95° C., protease digestion at 40C for 15 minutes and rinsing after each step (Bond Wash Solution). All other steps were performed at ambient temperature. Staining was performed with RNAscope 2.5 LS Reagent Kit—BROWN (Cat. No. 322100) or RNAscope 2.5 LS Reagent Kit—RED (Cat. No. 322150). Species-specific probes were applied and incubated at 42C for 120 minutes. Probes used were as follows: RNAscope 2.5 LS Positive Control Probe Human PPIB (Cat. No. 313908), RNAscope 2.5 LS Positive Control Probe Mouse PPIB (Cat. No. 313918), RNAscope 2.5 LS Negative Control Probe dapB (Cat. No. 312038), RNAscope 2.5 LS Human Angptl7 (Cat. No. 552818), RNAscope 2.5 LS Mouse Angotl7 (Cat. No. 552528). Chromogenic staining was performed using BOND Polymer Refine Detection (Cat. No. DS9800) or BOND Polymer Refine Red Detection (Cat. No. DS9390). After staining, slides were removed from the stainer, dehydrated and coverslipped. Slides stained with the Brown kit were coverslipped with Epredia Cytoseal XYL (Cat. No. 8312-4) and slides stained with the Red kit were coverslipped with Axell Crystal Mount (Cat. No. BMDM02).

Spatial enrichment localization score. For each spot x, the spatial enrichment score (SES) was computed as Dt/(Dn+Dt) where Dn equals the distance between spot x and the nearest necrotic border and Dt equals the distance between spot x and the nearest tumor border (Dt). Accordingly, SES(x) varies from 0 to 1: equal to 0 for perfect localization to the tumor border, and 1 for perfect localization to the necrotic border. Distances calculated from QuPath were exported to R and analyzed using a custom script. A non-parametric Kruskal-Wallis test was applied to evaluate statistical significance between different SES distributions.

LC/MS Plasma Proteomics.

Sample Preparation: Each plasma sample was depleted of high-abundance proteins by injecting 80 μL of the sample onto a Michrom Bioresources Paradigm HPLC equipped with an Agilent human MARS-6 depletion column. Unbound material eluting from the column, as observed on a UV detector, was collected and the protein concentration was determined. The volume corresponding to 100 μg of protein was subjected to the reduction of disulfide bonds by adding TCEP (tris(2-carboxyethyl) phosphine to a final concentration of 5 mM and incubating at room temperature with vortexing for 15 min. Protein alkylation was carried out by adding 2-chloroacetamide to a final concentration of 10 mM and incubating at room temperature for 30 min. The volume of each sample was reduced to approximately 100 μL by vacuum centrifugation and methanol/chloroform precipitation was carried out. Protein pellets were washed with methanol and resuspended in 50 mM HEPES pH 8.7. Proteolytic digestion was initiated with the addition of 1 μg of Lys-C and incubating at room temperature with low vortexing for 2 hours, followed by the addition of 1 μg of trypsin and incubating overnight at 37° C. on an orbital shaker set at 700 rpm.

TMT Labeling: Thermo Scientific TMTpro-16plex reagent was brought to room temperature and resuspended in 20 μL of anhydrous acetonitrile and vortexed for 15 min. Labeling was carried out by adding 20 μL of each TMT reagent to its assigned sample and vortexing the samples occasionally at room temperature for 1 hour. A “label check” was performed by combining 2 μL from each labeled sample into one tube, removing the acetonitrile by vacuum centrifugation, desalting the sample on a Harvard Apparatus C18 ultra-micro spin column, and analyzing the desalted TMT-labeled peptides by LC-ESI-MS/MS. After data analysis (see below), the peptide to spectrum match (PSM) results were analyzed and labeling efficiency was determined to be greater than 98%. After this labeling check, hydroxlyamine was added to each labeled sample to a concentration of 0.5% in order to fully quench the labeling reaction. All samples were combined equally by correcting for the total protein abundances measured for each sample in the labeling check analysis. The equalized pool was subjected to vacuum centrifugation to remove acetonitrile and then desalted on a Waters SepPack C18 (3cc, 200 mg) cartridge. The desalted elution was split into equal fractions and taken to dryness. One of the fractions was injected onto a Thermo Scientific Vanquish HPLC equipped with an Agilent 2.1 mm×150 mm C18 Extend column and fractionated into a 96-well plate using basic reverse-phase conditions. The 96 fractions were concatenated into 24 pools (pool 1: fractions 1, 25, 49, 73; pool 2: fractions 2, 26, 50, 74; etc.) that were taken to dryness and each pool was analyzed by LC-ESI-MS/MS.

LC-ESI-MS/MS: The generated basic reverse phase fractions were brought up in 20 μL of 2% acetonitrile in 0.1% formic acid and 5 μL was analyzed by LC/ESI MS/MS with a Thermo Scientific Easy1200 nLC (Thermo Scientific, Waltham, MA) coupled to a tribrid Orbitrap Eclipse with FAIMS (field asymmetric ion mobility spectrometry) mass spectrometer (Thermo Scientific, Waltham, MA). In-line de-salting was accomplished using a reversed-phase trap column (100 μm×20 mm) packed with Magic C18AQ (5-μm, 200 Å resin; Michrom Bioresources, Auburn, CA) followed by peptide separations on a reversed-phase column (75 μm×270 mm) packed with ReproSil-Pur C18AQ (3-μm, 120 Å resin; Dr. Maisch, Baden-Würtemburg, Germany) directly mounted on the electrospray ion source. A 120-minute gradient from 4% to 44% B (80% acetonitrile in 0.1% formic acid) at a flow rate of 300 nL/minute was used for chromatographic separations. A spray voltage of 2300 V was applied to the electrospray tip and the FAIMS source used varied compensation voltages of −40, −60, −80 while the Orbitrap Eclipse instrument was operated in the data-dependent mode. MS survey scans were in the Orbitrap (Normalized AGC target value 300%, resolution 120,000, and max injection time 50 ms) with a 3 sec cycle time and MS/MS spectra acquisition were detected in the Orbitrap (Normalized AGC target value of 250%, resolution 50,000 and max injection time 100 ms) using HCD activation with a normalized collision energy of 35%. Selected ions were dynamically excluded for 60 seconds after a repeat count of 1.

Data Analysis: Data analysis was performed using Proteome Discoverer 2.4 (Thermo Scientific, San Jose, CA). The data were searched against a Uniprot Human database (UP000005640, Dec. 1, 2019) that included common contaminants (cRAPome 2015). Searches were performed with settings for the proteolytic enzyme trypsin. Maximum missed cleavages were set to 2. The precursor ion tolerance was set to 10 ppm and the fragment ion tolerance was set to 0.6 Da. Dynamic peptide modifications included oxidation (+15.995 Da on M). Dynamic modifications on the protein terminus included acetyl (+42.−11 Da on N-terminus), Met-loss (−131.040 Da on M) and Met-loss+Acetyl (−89.030 Da on M) and static modifications TMTpro (+304.207 on N-terminus and K), carbamidomethyl (+57.021 on C). Sequest HT was used for database searching. All search results were run through Percolator for peptide validation and results were filter to a 1% false discovery rate. To delineate differential expression proteins, both pairwise two-sample t tests and Wilcoxon rank-sum test were conducted (R package).

Quantification and Statistical Analysis. Bars are presented as mean±SD. Graphs were created and statistical tests conducted in GraphPad Prism 8. Non-parametric tests were used when data were not normally distributed or when the median was a better representation of the sample than the mean. For animal experiments, each animal was considered a biological replicate. For in vitro experiments, experiments using cell lines on different days were considered biological replicates. P-values were denoted as follows: *P<0.05, **P<0.01, ***P<0.001, and ****P<0.0001.

Data, Materials, and Software Availability. Raw RNA-sequencing data have been deposited on the National Center for Biotechnology Information (NCBI) Sequence Read Archive (SRA) (BioProjects: PRJNA925873 (77), PRJNA925929 (78), PRJNA928719 (79); for necrotic core vs. rim, Angptl7 K_{D} vs. NT, and HUVEC Angptl7 treatment vs. control, respectively). RNA-seq analysis code: The RNAseq analysis code used to analyze tumor xenograft samples are available on Zenodo: DOI: 10.5281/zenodo.7574251, and future versions will be maintained on github: https://github.com/bkrajina/RNA_seq_xenograft_analysis. Qupath spatialanalysis code: DOI: 10.5281/zenodo.7574395.

### Example 2

This Example discloses administering an inhibitor of angiopoietin-like 7 to a subject with cancer.

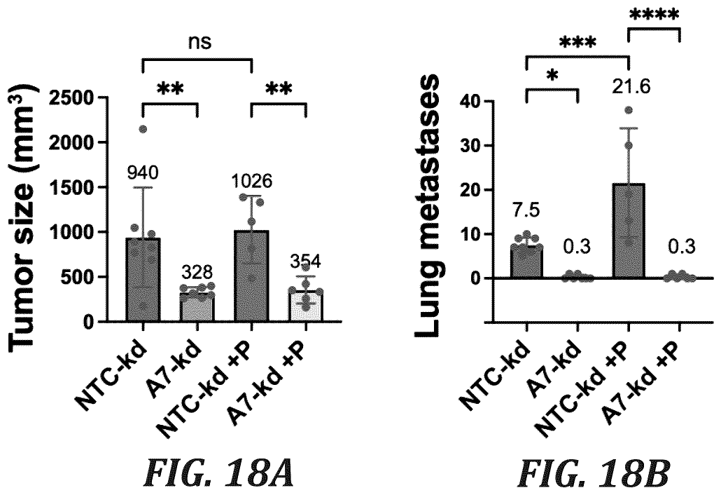

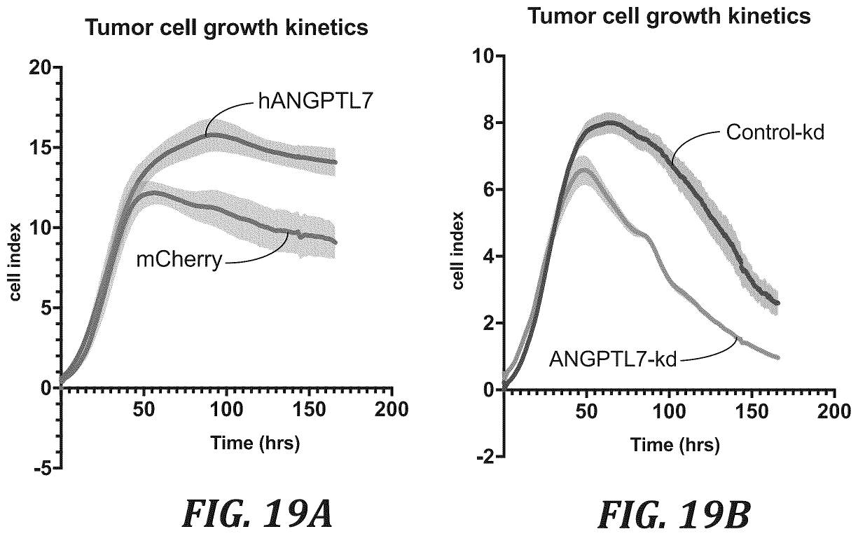

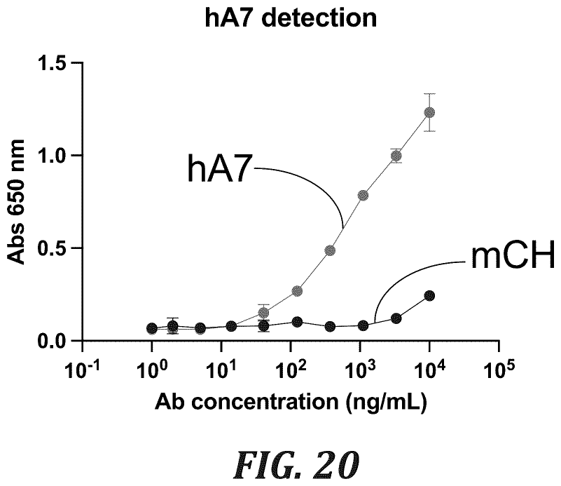

A subject with cancer will first be identified. The subject will have elevated angiopoietin-like 7 in tumor tissue or an elevated angiopoietin-like 7 in blood. The inhibitor of angiopoietin-like 7 will be administered to the subject. In some embodiments, the inhibitor of angiopoietin-like 7 is a monoclonal angiopoietin-like 7 antibody. In some embodiments, the inhibitor of angiopoietin-like 7 is a polyclonal angiopoietin-like 7 antibody. FIG. 20 (demonstrating that the Angptl7 antibody (PA536575) binds to native human Angptl7 in cell-based assays). Following administration of the inhibitor of angiopoietin-like 7, the level of angiopoietin-like 7 expression on the tumor will be decreased. Following administration of the inhibitor of angiopoietin-like 7, the activity level of angiopoietin-like 7 in the tumor will be decreased. The decrease in the tumor's level of angiopoietin-like 7 will decrease tumor cell proliferation in the tumor core. The tumor core is nutrient limited. As demonstrated in FIG. 19, angiopoietin-like 7 is necessary and sufficient for maximal tumor growth in nutrient limited conditions. The decrease in the tumor's level of angiopoietin-like 7 will decrease vascular leakiness in the tumor core. The decrease in the tumor's level of angiopoietin-like 7 will decrease necrosis formation in the tumor core. The decrease in the tumor's level of angiopoietin-like 7 will decrease metastasis formation (FIGS. 1-6). The decrease in the tumor's level of angiopoietin-like 7 will decrease circulating tumor cells (CTCs) (FIGS. 1-6). The decrease in the tumor's level of angiopoietin-like 7 will increase drug penetration into the tumor core. The decrease in the tumor's level of angiopoietin-like 7 will increase treatment effect of a second anti-cancer therapeutic agent. Decreasing tumor cell proliferation, necrosis formation, metastasis formation, and CTCs will increase the survival of a subject with cancer. Increasing drug penetration will increase treatment effect of a second anti-cancer therapeutic agent. The data illustrated in FIG. 18 demonstrate that angiopoietin-like 7 improve the effect of paclitaxel to reduce tumor size (i.e., proliferation) and lung metastases. From these data, it naturally follows that decreasing necrosis will increase perfusion to the tumor core (Jain R K. Barriers to drug delivery in solid tumors. Sci Am. 1994 July; 271(1):58-65; Jain R K. Delivery of molecular and cellular medicine to solid tumors. Adv Drug Deliv Rev. 2012 Dec. 1; 64 (Suppl):353-365).

While illustrative embodiments have been illustrated and described, it will be appreciated that various changes can be made therein without departing from the spirit and scope of the invention.

The embodiments of the invention in which an exclusive property or privilege is claimed are defined as follows:

1. A method of inhibiting necrotic core formation in a tumor to reduce the risk of metastatic dissemination from the tumor core, the method comprising:
(a) determining a level of circulating tumor cells (CTCs) in a biological sample from a cancer patient;
(b) comparing the level of CTCs in the biological sample to a CTC reference level, if the level of CTCs in the biological sample is not greater than the CTC reference level, repeat steps (a) and (b) until the level of CTCs from the biological sample is greater than the CTC reference level;
(c) identifying the cancer patient having an increased risk of metastatic dissemination if the level of CTCs from the biological sample is greater than the CTC reference level; and
(d) administering an Angptl7 inhibitor to the cancer patient having an increased level of CTCs from the biological sample compared to the CTC reference level, wherein the Angptl7 inhibitor would reduce necrotic core formation and prevent metastatic dissemination.

2. The method of claim 1, wherein the Angptl7 inhibitor is an Angptl7 antibody.

3. The method of claim 1, wherein the sample is a blood sample.

4. The method of claim 1, wherein the Angplt7 antibody is administered intravenously.

5. The method of claim 4, wherein the Angptl7 antibody is administered intravenously for 60 minutes once every 4 weeks at a dose of 15 mg/kg.

6. The method of claim 1, wherein the method further comprises administering a second therapeutic agent, wherein the second therapeutic agent is an anti-cancer therapeutic agent.

7. A method of increasing survival of a cancer patient, the method comprising administering a therapeutically effective amount of a composition to the cancer patient, the composition comprising an angiopoietin-like 7 (Angptl7) inhibitor and a pharmaceutically acceptable carrier, wherein the Angptl7 inhibitor and the pharmaceutically acceptable carrier are administered as a unit dosage.

8. The method of claim 7, wherein the Angptl7 inhibitor is an Angptl7 antibody.

9. The method of claim 7, wherein the composition is administered intravenously.

10. The method of claim 9, wherein the composition is administered intravenously for 60 minutes once every 4 weeks at a dose of 15 mg/kg.

11. The method of claim 7, wherein the composition further comprises a second therapeutic agent, wherein the second therapeutic agent is an anti-cancer therapeutic agent.

12. The method of claim 7, wherein the composition reduces expression of Angptl7 in a tumor.

13. The method of claim 7, wherein the composition results in preventing at least one indication of aggressive tumor cell behavior.

14. The method of claim 13, wherein the at least one indication of aggressive tumor cell behavior comprises necrosis formation in the tumor core.

15. The method of claim 13, wherein the at least one indication of aggressive tumor cell behavior comprises metastasis formation.

16. The method of claim 13, wherein the at least one indication of aggressive tumor cell behavior comprises an increase in circulating tumor cells (CTCs).

17. A method for reducing tumor cell proliferation in a cancer patient, the method comprising the steps of:
(a) collecting a sample from a cancer patient;
(b) detecting an angiopoietin-like 7 (Angptl7) expression level in the sample collected from the cancer patient;
(c) comparing the Angptl7 expression level in the sample collected from the cancer patient to an Angptl7 reference level; and
(d) administering to the cancer patient a therapeutically effective dose of an Angptl7 inhibitor, if the Angptl7 expression level in the sample collected from the cancer patient is greater compared to the Angptl7 reference level, wherein inhibiting Angptl7 expression and/or function reduces at least one indication of tumor cell proliferation.

18. The method of claim 17, wherein the Angptl7 inhibitor is an Angptl7 antibody.

19. The method of claim 17, wherein the at least one indication of tumor cell proliferation comprises necrosis formation in the tumor core, metastasis formation, or an increase in circulating tumor cells (CTCs)

20. The method of claim 17, wherein the method further comprises administering a second therapeutic agent, wherein the second therapeutic agent is an anti-cancer therapeutic agent.
